(19) United States
(12) Patent Application Publication
(10) Pub. No.: US 2012/0172369 A1
(43) Pub. Date: Jul. 5, 2012

(54) INHIBITORS OF DIACYLGLYCEROL ACYLTRANSFERASE

(76) Inventors: Pauline C. Ting, New Providence, NJ (US); Joe F. Lee, Brooklyn, NY (US); Robert G. Aslanian, Rockaway, NJ (US)

(21) Appl. No.: 13/395,589

(22) PCT Filed: Sep. 3, 2010
(86) PCT No.: PCT/US10/47765

§ 371 Date: Mar. 12, 2012

Related U.S. Application Data
(60) Provisional application No. 61/242,089, filed on Sep. 14, 2009.

(51) Int. Cl. A61K 31/506; C07D 409/14; C07D 405/12; A61K 31/496; A61P 3/06; A61K 31/443; A61K 31/444; A61K 31/4525; A61P 3/04; A61P 3/10; C07D 405/14; A61K 31/4545

(52) U.S. Cl. 514/252.14; 544/364; 546/194; 544/331; 546/284.1; 546/256; 546/196; 546/276.4; 544/295; 514/253.11; 514/318; 514/275; 514/337; 514/333; 514/320

(57) ABSTRACT

The present invention relates to novel heterocyclic compounds as diacylglycerol acyltransferase (DGAT) inhibitors, pharmaceutical compositions comprising the heterocyclic compounds and the use of the compounds for treating or preventing a cardiovascular disease, a metabolic disorder, obesity or an obesity-related disorder, diabetes, dyslipidemia, a diabetic complication, impaired glucose tolerance or impaired fasting glucose. An illustrative compound of the invention is shown below:

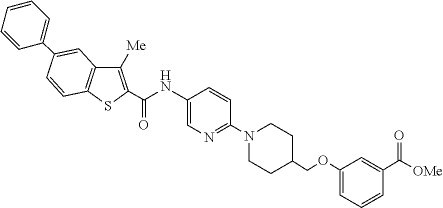

### FIELD OF THE INVENTION

The present invention relates to certain heterocyclic compounds useful as diacylglycerol acyltransferase (“DGAT”) inhibitors, especially diacylglycerol acyltransferase 1 (“DGAT1”) inhibitors, pharmaceutical compositions containing the compounds, and methods of treatment using the compounds and compositions to treat or prevent various diseases including cardiovascular disease, dyslipidemia, obesity and diabetes (e.g., Type 2 diabetes).

### BACKGROUND OF THE INVENTION

There is a need for additional ways of treating diseases associated with metabolic syndrome such as, for example, dyslipidemia, cardiovascular disease, obesity and diabetes (e.g., Type 2 diabetes).

Triglycerides or triacylglycerols are the major form of energy storage in eukaryotic organisms. In mammals, these compounds are primarily synthesized in three tissues: the small intestine, liver, and adipocytes. Triglycerides or triacylglycerols support the major functions of dietary fat absorption, packaging of newly synthesized fatty acids and storage in fat tissue (see Subauste and Burant, Current Drug Targets—Immune, Endocrine & Metabolic Disorders (2003) 3, pp. 263-270).

Diacylglycerol O-acyltransferase, also known as diglyceride acyltransferase or DGAT, is a key enzyme in triglyceride synthesis. DGAT catalyzes the final and rate-limiting step in the triacylglycerol synthesis from 1,2-diacylglycerol (DAG) and long chain fatty acyl CoA as substrates. Thus, DGAT plays an essential role in the metabolism of cellular diacylglycerol and is critically important for triglyceride production and energy storage homeostasis (see Mayorek et al, European Journal of Biochemistry (1989) 182, pp. 395-400).

Two forms of DGAT have been cloned and are designated DGAT1 and DGAT2 [see Cases et al, Proceedings of the National Academy of Science, USA (1998) 95, pp. 13018-13023, Lardizabal et al, Journal of Biological Chemistry (2001) 276, pp. 38862-38869 and Cases et al, Journal of Biological Chemistry (2001) 276, pp. 38870-38876]. Although both enzymes utilize the same substrates, there is no homology between DGAT1 and DGAT2. Both enzymes are widely expressed however some differences do exist in the relative abundance of expression in various tissues.

Disorders or imbalances in triglyceride metabolism, both absorption as well as de novo synthesis, have been implicated in the pathogenesis of a variety of disease risks. These include obesity, insulin resistance syndrome, Type II diabetes, dyslipidemia, metabolic syndrome (syndrome X) and coronary heart disease [see Kahn, Nature Genetics (2000) 25, pp. 6-7, Yanovski and Yanovski, New England Journal of Medicine (2002) 346, pp. 591-602, Lewis et al, Endocrine Reviews (2002) 23, pp. 201, Brazil, Nature Reviews Drug Discovery (2002) 1, pp. 408, Malloy and Kane, Advances in Internal Medicine (2001) 47, pp. 111, Subauste and Burant, Current Drug Targets—Immune, Endocrine & Metabolic Disorders (2003) 3, pp. 263-270 and Yu and Ginsberg, Annals of Medicine (2004) 36, pp. 252-261]. Compounds that can decrease the synthesis of triglycerides from diacylglycerol by inhibiting or lowering the activity of the DGAT enzyme would be of value as therapeutic agents for the treatment of diseases associated with abnormal metabolism of triglycerides.

Known inhibitors of DGAT include: dibenzoxazepinones (see Ramharack et al, EP1219716 and Burrows et al, 26th National Medicinal Chemistry Symposium (1998) poster C-22), substituted amino-pyrimidino-oxazines (see Fox et al, WO2004047755), chalcones such as xanthohumol (see Tabata et al, Phytochemistry (1997) 46, pp. 683-687 and Casaschi et al, Journal of Nutrition (2004) 134, pp. 1340-1346), substituted benzyl-phosphonates (see Kurogi et al, Journal of Medicinal Chemistry (1996) 39, pp. 1433-1437, Goto et al, Chemistry and Pharmaceutical Bulletin (1996) 44, pp. 547-551, Ikeda et al, Thirteenth International Symposium on Athersclerosis (2003), abstract 2P-0401, and Miyata et al, JP 2004067635), aryl alkyl acid derivatives (see Smith et al, WO2004100881 and US20040224997), furan and thiophene derivatives (see WO2004022551), pyrrolo[1,2b]pyridazine derivatives (see Fox et al, WO2005103907), and substituted sulfonamides (see Budd Haeberlein and Buckett, WO20050442500).

Also known to be inhibitors of DGAT are: 2-bromo-palmitic acid (see Colman et al, Biochimica et Biophysica Acta (1992) pp. 1125, 203-9), 2-bromo-octanoic acid (see Mayorek and Bar-Tana, Journal of Biological Chemistry (1985) 260, pp. 6528-6532), roselipins (see Noriko et al, (Journal of Antibiotics (1999) 52, pp. 815-826), amidepsin (see Tomoda et al, Journal of Antibiotics (1995) 48, pp. 42-7), isochromophilone, prenylflavonoids (see Chung et al, Planta Medica (2004) 70, v58-260), polyacetylenes (see Lee et al, Planta Medica (2004) 70, pp. 97-200), cochlioquinones (see Lee et al, Journal of Antibiotics (2003) 56, pp. 967-969), tanshinones (see Ko et al, Archives of Pharmaceutical Research (2002) 25, pp. 446-448), gemfibrozil (see Zhu et al, Atherosclerosis (2002) 164, pp. 221-228), and substituted quinolones (see Ko et al, Planta Medica (2002) 68, pp. 1131-1133). Also known to be modulators of DGAT activity are antisense oligonucleotides (see Monia and Graham, US20040185559).

Particular mention is made to PCT publication WO 2007/060140 (published May 31, 2007; applicant: F. Hoffmann-La Roche AG). Claim 1 therein discloses compounds of the formula:

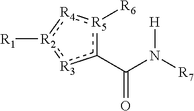

wherein R₁, R₂, R₃, R₄, R₅, R₆ and R₇ are described.

DGAT inhibitors have been described. See, for example, PCT publication US 2007/0244096 (published Oct. 31, 2007; applicant: Japan Tobacco). Claim 1 therein discloses compounds of the formula:

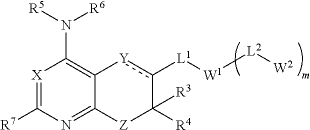

wherein R³, R⁴, R⁵, R⁶, R⁷, X, Y, Z, L¹, L², W¹, W² and in are described. WO 2007/126957 (published Nov. 8, 2007; applicant: Novartis Pharma). Claim 1 therein discloses compounds of the formula:

A-L1-B—C-D-L2-E

wherein A, L1, B, C, D, L2 and E are described.

WO 2008/067257 (published Jun. 5, 2008; applicant: Abbott Laboratories) discloses, in claim 1, compounds of the formula:

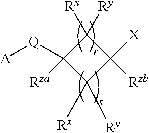

wherein A, Q, X, R^{x}, R^{y}, R^{za}, R^{zb}, r and s are described therein.

Commonly owned U.S. provisional patent applications, Ser. Nos. 61/115,991, 61/115,995, 61/116,000, 61/115,982, 61/115,985 and 61/115,987, all filed Nov. 19, 2008, and 61/161,212 filed Mar. 18, 2009 also describe DGAT inhibitors.

A need exists in the art, however, for additional DGAT inhibitors that have efficacy for the treatment of metabolic disorders such as, for example, obesity, Type II diabetes mellitus and metabolic syndrome.

### SUMMARY OF THE INVENTION

In an embodiment, this invention discloses a compound, or pharmaceutically acceptable salts, solvates, ester or prodrugs of said compound, or pharmaceutically acceptable salts, solvates or esters of said prodrug, the compound being represented by the general formula I:

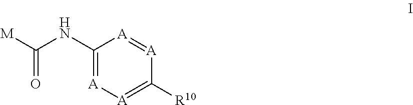

wherein:
each A is independently selected from C(R⁴) or N;
M is a bicyclic moiety selected from:

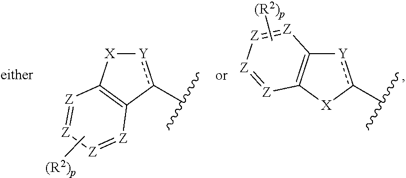

wherein each Z is independently selected from C(R³) or N;
each X is independently selected from C, O, N, or S;
each Y is independently selected from C(R³) or N;
p is 1-4;
[symbol] refers to an optional bond; and
R² is independently selected from H, alkyl-, haloalkyl-, halo, alkoxy-, (alkoxy)alkyl-, haloalkoxy-, cycloalkyl-, (cycloalkyl)alkyl-, aryl-, (aryl)alkyl-, heteroaryl-, (heteroaryl)alkyl-, heterocyclyl-, and (heterocyclyl)alkyl-, wherein each of said alkyl, haloalkyl, alkoxy, (alkoxy)alkyl, heteroaryl-, (heteroaryl)alkyl-, heterocyclyl-, and (heterocyclyl)alkyl can be independently unsubstituted or optionally independently substituted with one or more moieties which are the same or different, each substituent being independently selected from the group consisting of alkyl, alkoxy, alkoxyalkyl, haloalkoxy, alkenyl, alkynyl, cycloalkyl, cycloalkylalkyl, cycloalkenyl, cycloalkenylalkyl, aryl, arylalkyl, heteroaryl, heteroarylalkyl, halo, —CN, —OR^{c}, ═O, —C(O)R^{c}, —C(O)OR^{c}, —C(O)N(R^{c})(R^{d}), —SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}), —N(R^{c})(R^{d}), -alkyl-N(R^{c})(R^{d}), —N(R^{c})C(O)R^{d}, —CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d}, —N(R^{c})S(O)₂N(R^{d})(R^{b}), —N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{C}, —N₃, —NO₂ and —S(O)₂R^{c}, wherein each R^{b}, R^{c} and R^{d} is independently selected;
   R¹⁰ is either (i) a 4-8 membered heterocyclyl ring having from 1 to 3 ring N atoms, or (ii) a bicyclic heterocyclyl ring having from 1 to 3 ring N atoms, or (iii) an aryl group, or (iv) a heteroaryl group,
wherein each of said aryl or heteroaryl group for R¹⁰ independently is unsubstituted or optionally independently substituted with one or more G moieties wherein said G moieties can be the same or different, each G moiety being independently selected from the group shown below,
and further wherein each of said heterocyclyl ring and bicyclic heterocyclyl ring for R¹⁰ independently is unsubstituted or optionally substituted, off of either (i) a ring N atom or (ii) a ring carbon atom on said heterocyclyl ring or said bicyclic heterocyclyl ring, with one or more G moieties wherein said G moieties can be the same or different, each G moiety being independently selected from the group consisting of:
   (a)

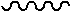

(b)

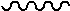

(c)

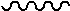

(d)

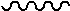

(e)

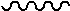

(f)

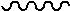

(g)

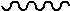

(h)

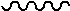

off of only C and not off of N;
(i)

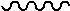

off of only C and not off of N;
(j)

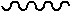

(k)

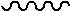

(l)

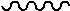

off of only C and not off of N;
(m)

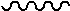

off of only C and not off of N;
(n) an oxo group off of only C and not off of N; and
(O)

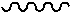

wherein R^{a} is selected from the group consisting of hydrogen, hydroxy, CN, halo, alkyl, alkenyl, alkynyl, aryl, heteroaryl, heterocyclyl, cycloalkyl or spirocyclyl, wherein each of said alkyl, alkenyl, alkynyl, aryl, heteroaryl, heterocyclyl and cycloalkyl is unsubstituted or optionally independently substituted with one or more moieties which are the same or different, each moiety being selected independently from the group consisting of O-haloalkyl, S-haloalkyl, CN, NO₂, CF₃, cycloalkyl, heterocyclyl, haloalkyl, aryl, heteroaryl, N-alkyl, N-haloalkyl, and N-cycloalkyl; alkyl, alkenyl, alkynyl, cycloalkylalkyl, cycloalkenyl, heterocyclylalkyl, aryl, arylalkyl, heteroaryl, heteroarylalkyl, halo, —OR^{c}, —C(O)R^{c}, C(O)OR^{c},
—C(O)N(R^{c})(R^{d}), —SF₅, —OSF₅, —Si(R^{c})₃, —SR^{c}, —S(O)N(R^{c})(R^{d}),
—CH(R^{c})(R^{d}), —S(O)₂N(R^{c})(R^{d}), —C(═NOR^{c})R^{d}, —P(O)(OR^{c})(OR^{d}),
—N(R^{c})(R^{d}), -alkyl-N(R^{c})(R^{d}), —N(R^{c})C(O)R^{d}, —CH₂—N(R^{c})C(O)R^{d},
—CH₂—N(R^{c})C(O)N(R^{d})(R^{b}), —CH₂—R^{c}; —CH₂N(R^{c})(R^{d}), —N(R^{c})S(O)R^{d},
—N(R^{c})S(O)₂R^{d}, —CH₂—N(R^{c})S(O)₂R^{d}, —N(R^{c})S(O)₂N(R^{d})(R^{b}),
—N(R^{c})S(O)N(R^{d})(R^{b}), —N(R^{c})C(O)N(R^{d})(R^{b}), —CH₂—N(R^{c})C(O)N(R^{d})(R^{b}), N(R^{c})C(O)OR^{d}, —CH₂—N(R^{c})C(O)OR^{d}, —S(O)R^{c}, ═NOR^{c}, —N₃, and
—S(O)₂R^{c};
wherein each R^{b}, R^{c} and R^{d} is independently selected;
R^{b} is H, lower alkyl, cycloalkyl, aryl, heteroaryl or heterocycloalkyl;
R^{c} is H, lower alkyl, cycloalkyl, aryl, heteroaryl or heterocycloalkyl;
R^{d} is H, lower alkyl, cycloalkyl, aryl, heteroaryl or heterocycloalkyl;
wherein each of said alkyl, cycloalkyl, aryl, heteroaryl or heterocycloalkyl in R^{b}, R^{c}, and R^{d} can be unsubstituted or optionally independently substituted with 1-2 substituents independently selected from halo, OH, NH₂, CF₃, CN, Oalkyl, NHalkyl, N(alkyl)₂ and Si(alkyl)₃;
   R³ is independently selected from H, alkyl, haloalkyl, cycloalkyl, and (cycloalkyl)alkyl-;
   R⁴ is independently selected from H, alkyl, haloalkyl, cycloalkyl, and (cycloalkyl)alkyl-;
   m is 1-3,
   n is 0-3, and
   n is 1-3.

The term “optional bond” means the bond may be present or absent. If it is present, the moiety [symbol] refers to a double bond, and if it is absent, the moiety [symbol] refers to a single bond.

The term “spirocyclyl” refers to a cyclic group substituted off the same carbon atom. Some non-limiting examples would be:

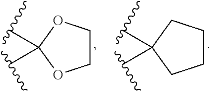

The term “oxo” refers to the moiety ═C(O) substituted off the same carbon atom.

The term “bicyclic heterocyclyl” refers to bicyclic compounds containing heteroatom as part of the ring atoms. A non-limiting example would be:

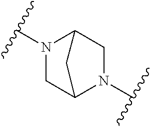

with no limitation as to the position of the heteroatom.

When a disubstituted moiety is shown with [symbol] on both sides, the attachment points are from left to right when looking at the parent formula, e.g. Formula I. Thus, for example, if the moiety:

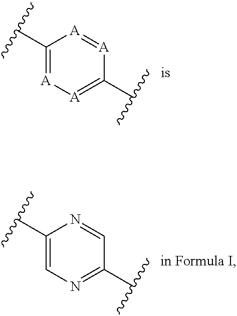

it means that the pyrazine ring is attached to NH on the left hand side and R¹⁰ on the right hand side in Formula I.

In another aspect, this invention provides compositions comprising at least one compound of Formula I.

In another aspect, this invention provides pharmaceutical compositions comprising at least one compound of Formula I and at least one pharmaceutically acceptable carrier.

In another aspect, this invention provides a method of treating diabetes in a patient in need of such treatment using therapeutically effective amounts of at least one compound of Formula I, or of a composition comprising at least one compound of Formula I.

In another aspect, this invention provides a method of treating diabetes in a patient in need of such treatment, e.g., Type 2 diabetes, using therapeutically effective amounts of at least one compound of Formula I, or of a composition comprising at least one compound of Formula I.

In another aspect, this invention provides a method of treating metabolic syndrome in a patient in need of such treatment, using therapeutically effective amounts of at least one compound of Formula I, or of a composition comprising at least one compound of Formula I.

In another aspect, this invention provides a method of inhibiting DGAT using therapeutically effective amounts of at least one compound of Formula I, or of a composition comprising at least one compound of Formula I.

In another aspect, this invention provides a method of inhibiting DGATI using therapeutically effective amounts of at least one compound of Formula I, or of a composition comprising at least one compound of Formula I.

### DESCRIPTION OF THE INVENTION

In an embodiment, the present invention discloses compounds of Formula I, or pharmaceutically acceptable salts, solvates, esters or prodrugs thereof.

The following embodiments (sometimes referred to as “another embodiment”) are independent of each other; different such embodiments can be independently selected and combined in various combinations. All such combinations should be considered as part of the invention.

In another embodiment, A is C(R⁴).

In another embodiment, A is N.

In another embodiment, one A is N and the other A moieties are C(R⁴).

In another embodiment, one A is C(R⁴) and the other A moieties are N.

In another embodiment, two A moieties are N and the other two A moieties are C(R⁴).

In another embodiment, Z is C(R³).

In another embodiment, Z is N.

In another embodiment, one Z is N and the other Z moieties are C(R³).

In another embodiment, one Z is C(R³) and the other Z moieties are N.

In another embodiment, two Z moieties are N and the other two Z moieties are C(R³).

In another embodiment, X is C.

In another embodiment, X is N.

In another embodiment, X is O.

In another embodiment, X is S.

In another embodiment, at least one X is O.

In another embodiment, at least one Y is N.

In another embodiment, one X is O and one other X is N.

In another embodiment, one X is O, one X is N and the other X is C(R³).

In another embodiment, Y is C(R³).

In another embodiment, Y is N.

In another embodiment, M is the moiety

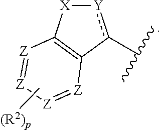

In another embodiment, M is the moiety

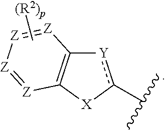

In another embodiment, R² is alkyl.

In another embodiment, R² is haloalkyl.

In another embodiment, R² is halo.

In another embodiment, R² is alkoxy.

In another embodiment, R² is (alkoxy)alkyl.

In another embodiment, R² is haloalkoxy.

In another embodiment, R² is cycloalkyl.

In another embodiment, R² is (cycloalkyl)alkyl.

In another embodiment, R² is aryl.

In another embodiment, R² is (aryl)alkyl.

In another embodiment, R² is heteroaryl.

In another embodiment, R² is (heteroaryl)alkyl.

In another embodiment, R² is heterocyclyl.

In another embodiment, R² is (heterocyclyl)alkyl.

In another embodiment, R² is heterocyclyl.

In another embodiment, R² is unsubstituted heterocyclyl.

In another embodiment, R² is 4-8 membered heterocyclyl, containing 1-3 heteroatoms which can be the same or different and is independently selected from the group consisting of N, O and S, wherein said heterocyclyl can be unsubstituted or optionally substituted, as defined earlier.

In another embodiment, R² is 3-7 membered heterocyclyl, containing 1-3 heteroatoms which can be the same or different and is independently selected from the group consisting of N, O and S, wherein said heterocyclyl can be unsubstituted or optionally substituted as defined earlier.

In another embodiment, R² is pyrrolidinyl, wherein said heterocyclyl can be unsubstituted or optionally substituted, as defined earlier.

In another embodiment, R² is piperidinyl, wherein said heterocyclyl can be unsubstituted or optionally substituted, as defined earlier.

In another embodiment, R² is piperazinyl, wherein said heterocyclyl can be unsubstituted or optionally substituted, as defined earlier.

In another embodiment, R² is morpholinyl, wherein said heterocyclyl can be unsubstituted or optionally substituted, as defined earlier.

In another embodiment, R² is thiamorpholinyl, wherein said heterocyclyl can be unsubstituted or optionally substituted as defined earlier.

In another embodiment, R² is azetidinyl, wherein said heterocyclyl can be unsubstituted or optionally substituted, as defined earlier.

In another embodiment, R² is azepinyl, wherein said heterocyclyl can be unsubstituted or optionally substituted as defined earlier.

In another embodiment, R² is oxazepinyl, wherein said heterocyclyl can be unsubstituted or optionally substituted, as defined earlier.

In another embodiment, R² is the moiety:

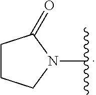

In another embodiment, R² is 4-8 membered heterocyclyl, containing 1-3 heteroatoms which can be the same or different and is independently selected from the group consisting of N, O and S, wherein said heterocyclyl can be unsubstituted or optionally substituted as defined earlier as defined earlier.

In another embodiment, R² is 4-8 membered heterocyclyl, containing 1-3 heteroatoms which can be the same or different and is independently selected from the group consisting of N, O and S, wherein said heterocyclyl can be unsubstituted or optionally substituted as defined earlier.

In another embodiment, R² is pyrrolidinyl, wherein said pyrrolidinyl can be unsubstituted or optionally substituted as defined earlier.

In another embodiment, R² is piperidinyl, wherein said piperidnyl can be unsubstituted or optionally substituted as defined earlier.

In another embodiment, R² is piperazinyl, wherein said piperazinyl can be unsubstituted or optionally substituted as defined earlier.

In another embodiment, R² is morpholinyl, wherein said morpholinyl can be unsubstituted or optionally substituted as defined earlier.

In another embodiment, R² is 4-8 membered heterocyclyl, containing 1-3 heteroatoms which can be the same or different and is independently selected from the group consisting of N, O and S, wherein said heterocyclyl is substituted with an aryl wherein said aryl can be unsubstituted or optionally substituted as defined earlier.

In another embodiment, R² is 4-8 membered heterocyclyl, containing 1-3 heteroatoms which can be the same or different and is independently selected from the group consisting of N, O and S, wherein said heterocyclyl is substituted with a phenyl wherein said phenyl can be unsubstituted or optionally substituted as defined earlier.

In another embodiment, R² is pyrrolidinyl, wherein said pyrroldinyl is substituted with a phenyl wherein said phenyl can be unsubstituted or optionally substituted as defined earlier.

In another embodiment, R² is piperidinyl, wherein said pyrroldinyl is substituted with a phenyl wherein said phenyl can be unsubstituted or optionally substituted as defined earlier.

In another embodiment, R² is piperazinyl, wherein said pyrroldinyl is substituted with a phenyl wherein said phenyl can be unsubstituted or optionally substituted as defined earlier.

In another embodiment, R² is morpholinyl, wherein said pyrroldinyl is substituted with a phenyl wherein said phenyl can be unsubstituted or optionally substituted as defined earlier.

In another embodiment, R³ is H.

In another embodiment, R³ is alkyl.

In another embodiment, R³ is lower alkyl.

In another embodiment, R³ is haloalkyl.

In another embodiment, R³ is cycloalkyl.

In another embodiment, R³ is (cycloalkyl)alkyl-.

In another embodiment, R⁴ is H.

In another embodiment, R⁴ is alkyl.

In another embodiment, R⁴ is lower alkyl.

In another embodiment, R⁴ is haloalkyl.

In another embodiment, R⁴ is cycloalkyl.

In another embodiment, R⁴ is (cycloalkyl)alkyl-

In another embodiment, R¹⁰ is a 4-8-membered heterocyclyl ring having from 1 to 3 ring N atoms, wherein said heterocyclyl ring is substituted off of a ring N atom.

In another embodiment, R¹⁰ is a 4-8-membered heterocyclyl ring having from 1 to 3 ring N atoms, wherein said heterocyclyl ring is substituted off of a ring carbon atom.

In another embodiment, R¹⁰ is a bicyclic heterocyclyl ring having from 1 to 3 ring N atoms, wherein said bicyclic heterocyclyl ring is substituted off of a ring N atom.

In another embodiment, R¹⁰ is a bicyclic heterocyclyl ring having from 1 to 3 ring N atoms, wherein said bicyclic heterocyclyl ring is substituted off of a ring carbon atom.

In another embodiment, R¹⁰ is a 4-8-membered heterocyclyl ring having from 1 to 3 ring N atoms, wherein said heterocyclyl ring is substituted with G, wherein G is as previously described.

In another embodiment, R¹⁰ is the moiety:

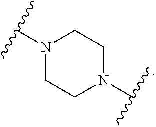

In another embodiment, R¹⁰ is the moiety:

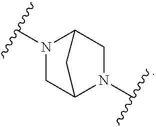

In another embodiment, R¹⁰ is the moiety:

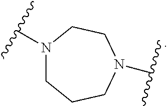

In another embodiment, R¹⁰ is the moiety:

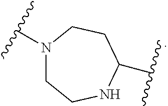

In another embodiment, R¹⁰ is a piperidinyl ring, wherein said piperidinyl ring is substituted with G, wherein G is as previously described.

In another embodiment, R¹⁰ is a piperazinyl ring, wherein said piperazinyl ring is with G, wherein G is as previously described.

In another embodiment, R¹⁰ is a diazepinyl ring, wherein said diazepinyl ring is substituted with G, wherein G is as previously described.

In another embodiment, R¹⁰ is a diazepinyl ring, wherein said diazepinyl ring is substituted with two G moieties, wherein G is as previously described.

In another embodiment, G is

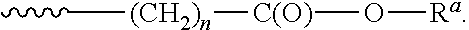

In another embodiment, G is

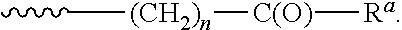

In another embodiment, G is

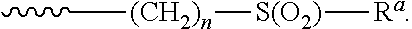

In another embodiment, G is

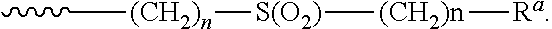

In another embodiment, G is

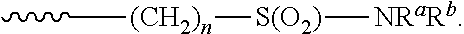

In another embodiment, G is

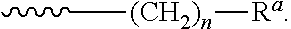

In another embodiment, G is

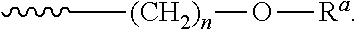

In another embodiment, G is

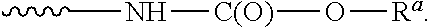

In another embodiment, G is

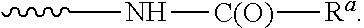

In another embodiment, G is

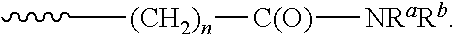

In another embodiment, G is

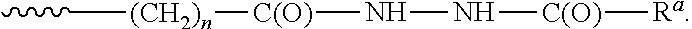

In another embodiment, G is

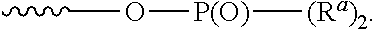

In another embodiment, G is

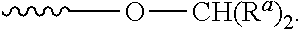

In another embodiment, G is an oxo group.

In another embodiment, G is

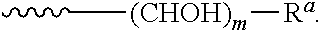

In another embodiment, G is a spirocyclyl group.

In another embodiment, G is the moiety:

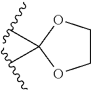

coming off of a carbon atom of R¹⁰.

In another embodiment, R^{a} is unsubstituted alkyl.

In another embodiment, R^{a} is alkyl substituted as previously described under formula

In another embodiment, R^{a} is unsubstituted aryl.

In another embodiment, R^{a} is aryl substituted as previously described under formula I.

In another embodiment, R^{a} is unsubstituted heteroaryl.

In another embodiment, R^{a} is heteroaryl substituted as previously described under formula I.

In another embodiment, R^{a} is unsubstituted cycloalkyl.

In another embodiment, R^{a} is cycloalkyl substituted as previously described under formula I.

In another embodiment, R^{a} is unsubstituted heterocyclyl.

In another embodiment, R^{a} is heterocyclyl substituted as previously described under formula I.

In another embodiment, R^{a} is hydroxy.

In another embodiment, R^{a} is cyano.

In another embodiment, R^{a} is halo.

In another embodiment, R^{a} is alkeny.

In another embodiment, R^{a} is alkynyl.

In another embodiment, R^{a} is alkoxyalkyl.

In another embodiment, R^{a} is aralkyl.

In another embodiment, R^{a} is haloalkyl.

In another embodiment, R^{a} is CF₃.

In another embodiment, R^{a} is phenyl substituted with one or more halo groups.

In another embodiment, R^{a} is heteroaryl.

In another embodiment, R^{a} is pyridyl.

In another embodiment, R^{a} is oxazolyl.

In another embodiment, R^{a} is oxadiazolyl.

In another embodiment, the moiety is:

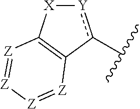

is

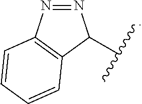

In another embodiment, the moiety is:

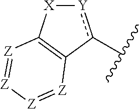

is

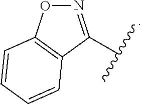

In another embodiment, the moiety is:

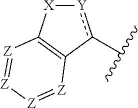

is

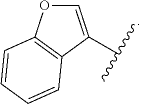

In another embodiment, the moiety is:

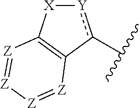

is

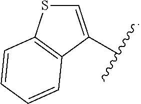

In another embodiment, the moiety is:

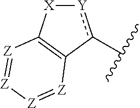

is

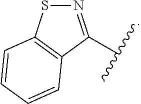

In another embodiment, the moiety is:

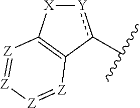

is

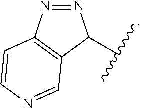

In another embodiment, the moiety is:

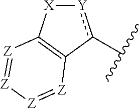

is

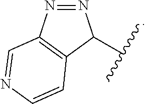

In another embodiment, the moiety is:

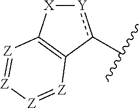

is

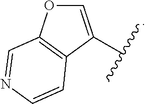

In another embodiment, the moiety is:

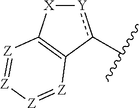

is

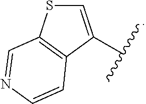

In another embodiment, the moiety is:

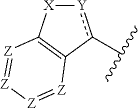

is

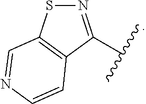

In another embodiment, the moiety is:

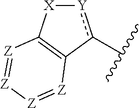

is

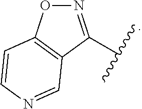

In another embodiment, the moiety is:

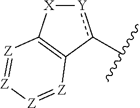

is

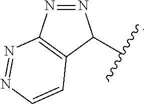

In another embodiment, the moiety is:

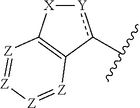

is

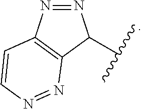

In another embodiment, the moiety is:

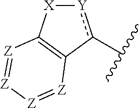

is

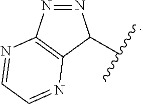

In another embodiment, the moiety is:

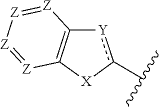

is

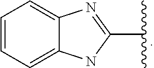

In another embodiment, the moiety is:

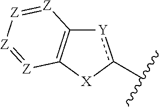

is

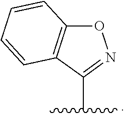

In another embodiment, the moiety is:

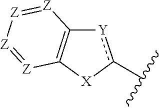

is

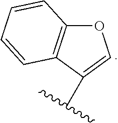

In another embodiment, the moiety is:

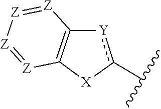

is

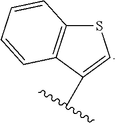

In another embodiment, the moiety is:

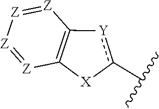

is

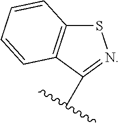

In another embodiment, the moiety is:

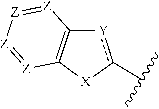

is

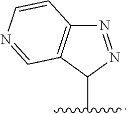

In another embodiment, the moiety is:

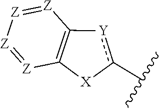

is

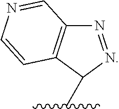

In another embodiment, the moiety is:

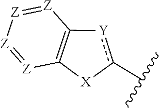

is

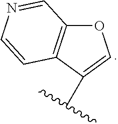

In another embodiment, the moiety is:

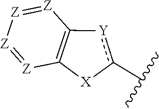

is

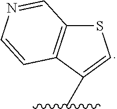

In another embodiment, the moiety is:

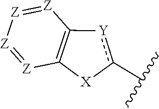

is

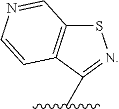

In another embodiment, the moiety is:

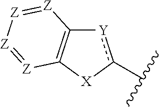

is

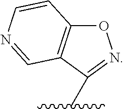

In another embodiment, the moiety is:

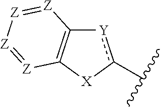

is

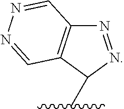

In another embodiment, the moiety is:

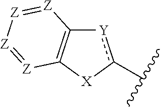

is

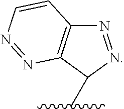

In another embodiment, the moiety is:

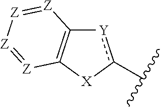

is

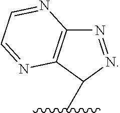

In another embodiment, in Formula I, the moiety:

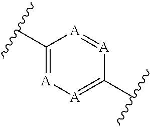

is selected from the group consisting of the following moieties:

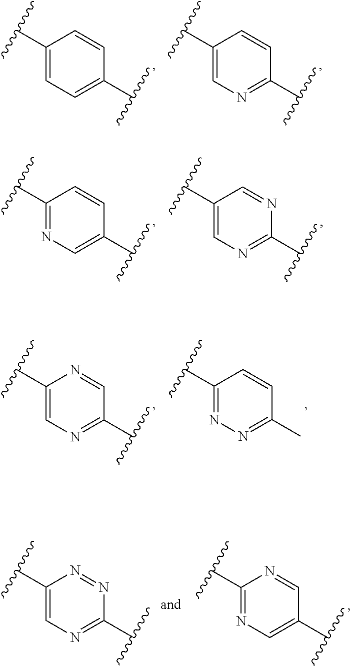

as well as any of their positional isomers.

In another embodiment, in Formula I, the moiety:

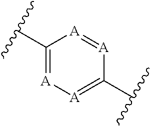

is

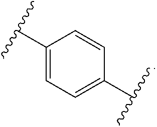

In another embodiment, in Formula I, the moiety:

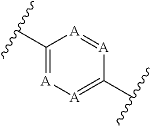

is

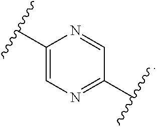

In another embodiment, in Formula I, the moiety:

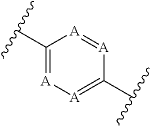

is

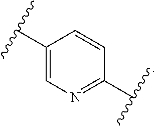

In another embodiment, in Formula I, the moiety:

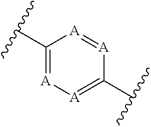

is

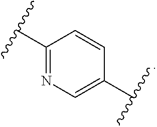

In another embodiment, in Formula I, the moiety:

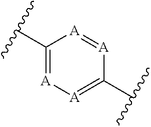

is

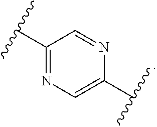

In another embodiment, p is 1.

In another embodiment of Formula I, wherein X, Y, Z, R², A, R¹⁰, R^{a} and the other moieties are independently selected, one X is N, a second X is C, and the third X is O, both Y are C, one A is N and the other A moieties are C(R⁴), R² is unsubstituted heterocyclyl, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A moieties are C(R⁴), R² is heterocyclyl substituted as described earlier, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both. Y are C, one A is N and the other A's are C(R⁴), R² is unsubstituted pyrrolidinyl, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is pyrrolidinyl substituted as described previously under Formula I, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted piperidinyl, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is piperidinyl substituted as described previously under Formula I, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted piperazinyl, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is piperazinyl substituted as described previously under Formula I, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted moepholinyl, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is morpholinyl substituted as described previously under Formula I, R¹⁰ is piperidinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted pyrrolidinyl, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is pyrrolidinyl as described earlier, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted piperidinyl, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is piperidinyl substituted as described earlier, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted piperazinyl, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is piperazinyl as described earlier, R¹⁰ is piperazinyl ring and R^{a} is as previously described.

In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is unsubstituted morpholinyl, R¹⁰ is piperazinyl ring and R^{a} is as previously described.

R¹⁰, In another embodiment of Formula I, wherein X, Y, Z, R², A, R¹⁰, R^{a} and the other moieties are independently selected, one X is N, a second X is C, and the third X is O, both Y are C, one A is N and the other A's are C(R⁴), R² is morpholinyl as described earlier, R¹⁰ is piperazinyl ring and R^{a} is as previously described.

In another embodiment of Formula I, wherein X, Y, Z, R², A, R¹⁰, R^{a} and the other moieties are independently selected, the moiety:

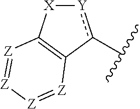

is

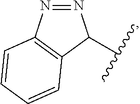

one A is N and the other A's are C(R⁴), R² is heterocyclyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

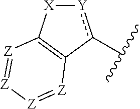

is

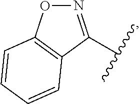

one A is N and the other A's are C(R⁴), R² is heterocyclyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

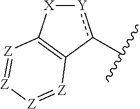

is

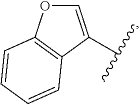

one A is N and the other A's are C(R⁴), R² is heterocyclyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

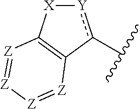

is

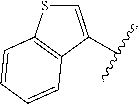

one A is N and the other A's are C(R⁴), R² is heterocyclyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

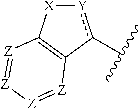

is

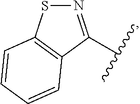

one A is N and the other A's are C(R⁴), R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

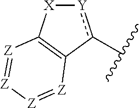

is

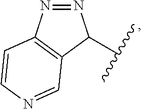

one A is N and the other A's are C(R⁴), R² is piperidinyl (unsubstituted or substituted as described earlier), R¹⁰ is piperazinyl ring and R^{a} is as previously described.

In another embodiment of Formula I, wherein X, Y, X, R², A, R¹⁰, R^{a} and the other moieties are independently selected, the moiety:

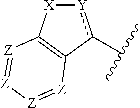

is

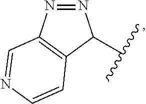

the moiety:

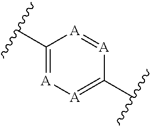

is

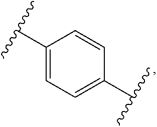

R² is piperazinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

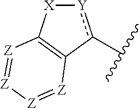

is

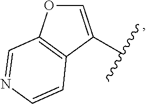

the moiety:

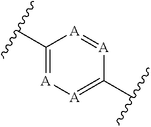

is

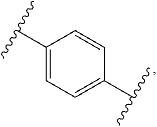

R² is morpholinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

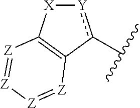

is

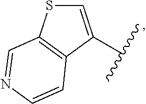

the moiety:

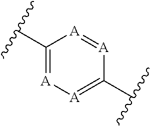

is

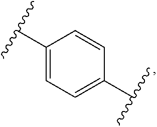

R² is pyrrolidinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

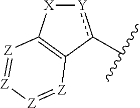

is

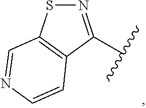

the moiety:

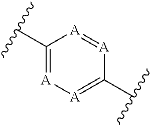

is

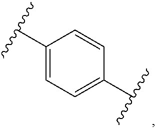

R² is piperidnyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

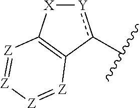

is

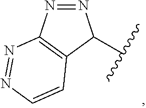

one A is N and the other A's are C, R² is piperidinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

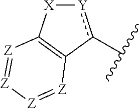

is

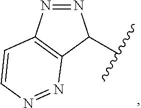

the moiety:

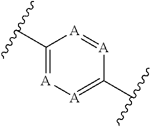

is

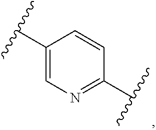

R² is piperazinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

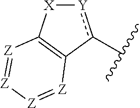

is

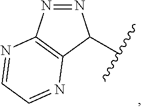

the moiety:

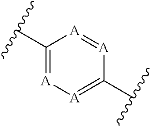

is

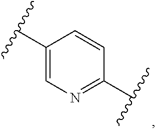

R² is morpholinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

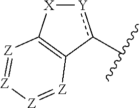

is

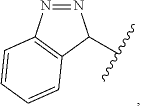

the moiety:

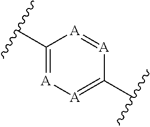

is

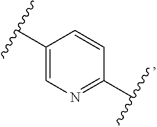

R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

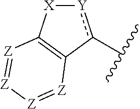

is

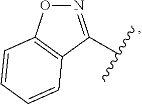

the moiety:

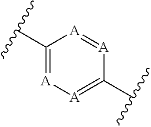

is

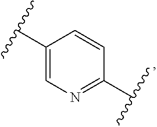

R² is piperidnyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

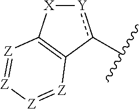

is

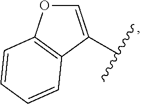

the moiety:

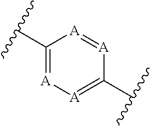

is

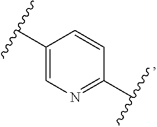

R² is piperazinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

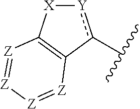

is

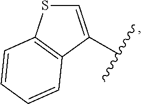

the moiety:

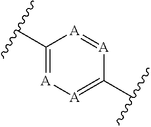

is

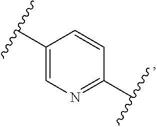

R² is morpholinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

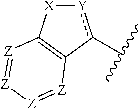

is

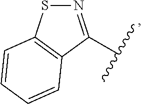

the moiety:

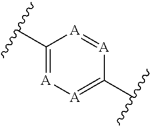

is

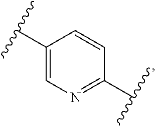

R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

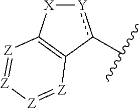

is

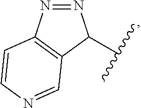

the moiety:

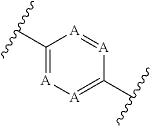

is

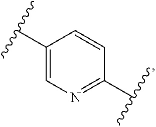

R² is piperidinyl (unsubstituted or substituted as described earlier), R¹⁰ is piperazinyl ring and R^{a} is as previously described.

In another embodiment of Formula I, wherein X, Y, R², A, R¹⁰, R^{a} and the other moieties are independently selected, the moiety:

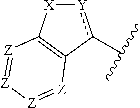

is

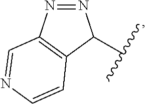

the moiety:

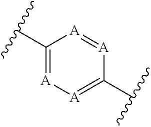

is

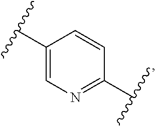

R² is piperazinyl (unsubstituted or substituted as described earlier), R¹⁰ is piperidinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

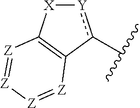

is

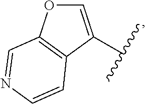

the moiety:

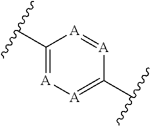

is

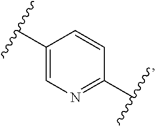

R² is morpholinyl (unsubstituted or substituted as described earlier), R¹⁰ is piperidinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

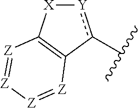

is

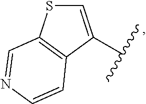

the moiety:

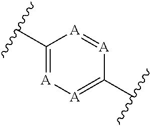

is

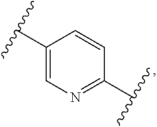

R² is pyrrolidinyl (unsubstituted or substituted as described earlier), R¹⁰ is piperazinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

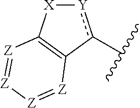

is

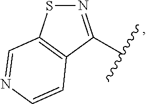

the moiety:

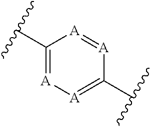

is

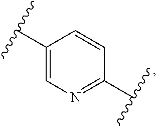

R² is piperidinyl (unsubstituted or substituted as described earlier), R¹⁰ is piperazinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

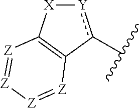

is

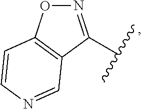

the moiety:

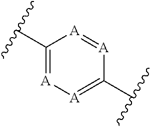

is

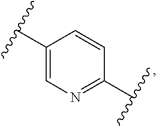

R² is piperazinyl (unsubstituted or substituted as described earlier), R¹⁰ is piperidinyl ring with —C(O)—O—R^{a}, and R^{a} is as previously described.

In another embodiment of Formula I, wherein X, Y, Z, R², A, R¹⁰, R^{a} and the other moieties are independently selected, the moiety:
the moiety

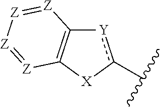

is:

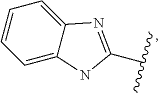

one A is N and the other A's are C(R⁴), R² is heterocyclyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

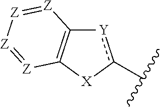

is:

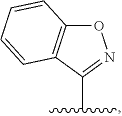

one A is N and the other A's are C(R⁴), R² is heterocyclyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

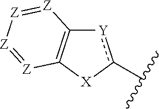

is:

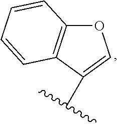

one A is N and the other A's are C(R⁴), R² is heterocyclyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

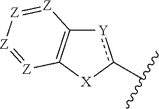

is:

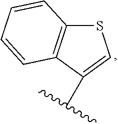

one A is N and the other A's are C(R⁴), R² is heterocyclyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

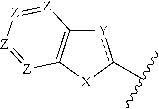

is:

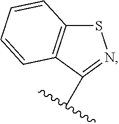

one A is N and the other A's are C(R⁴), R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

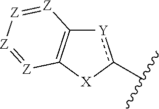

is:

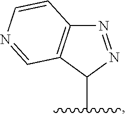

one A is N and the other A's are C(R⁴), R² is piperidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

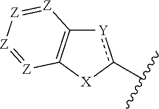

is:

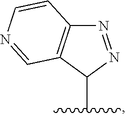

the moiety:

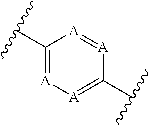

is

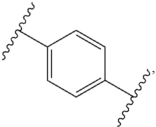

R² is piperazinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

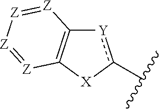

is:

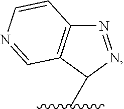

the moiety:

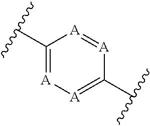

is

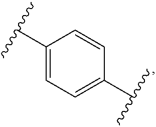

R² is morpholinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

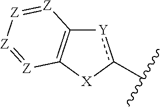

is:

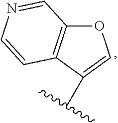

the moiety:

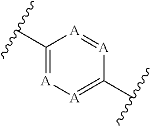

is

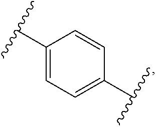

R² is pyrrolidinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

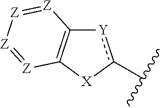

is:

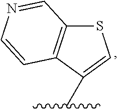

the moiety:

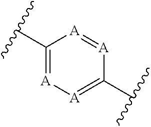

is

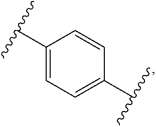

R² is piperidnyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

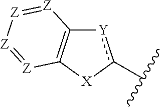

is:

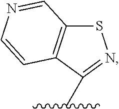

one A is N and the other A's are C, R² is piperidinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

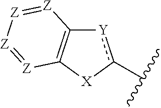

is:

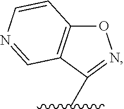

the moiety:

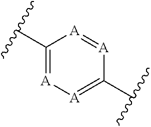

is

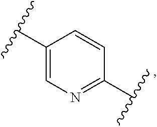

R² is piperazinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

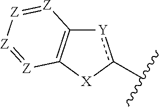

is:

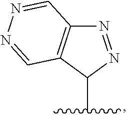

the moiety:

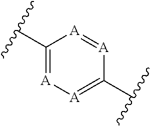

is

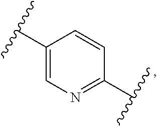

R² is morpholinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

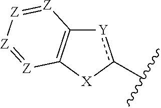

is:

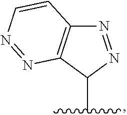

the moiety:

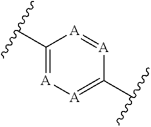

is

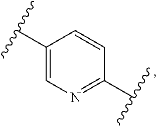

R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

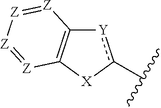

is:

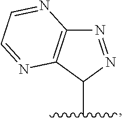

the moiety:

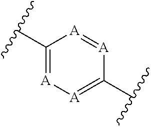

is

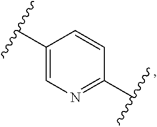

R² is piperidnyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

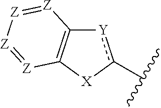

is:

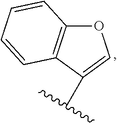

the moiety:

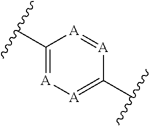

is

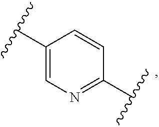

R² is piperazinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

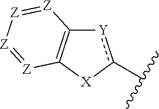

is:

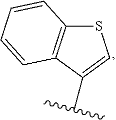

the moiety:

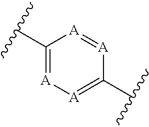

is

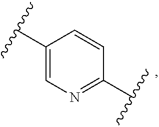

R² is morpholinyl (unsubstituted or substituted as described earlier), R¹⁰ is piperidinyl ring and R^{a} is as previously described.

In another embodiment of Formula I, wherein X, Y, Z, R², A, R¹⁰, R^{a} and the other moieties are independently selected, the moiety:

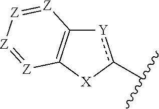

is:

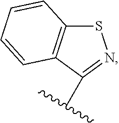

the moiety:

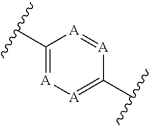

is

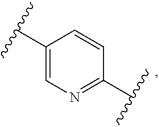

R² is pyrrolidinyl (unsubstituted or substituted as described earlier), R¹⁰ is piperazinyl ring and R^{a} is as previously described.

In another embodiment of Formula I, wherein X, Y, Z, R², A, R¹⁰, R^{a} and the other moieties are independently selected, the moiety:

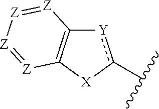

is:

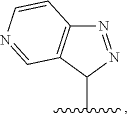

the moiety:

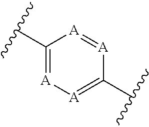

is

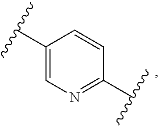

R² is piperidinyl (unsubstituted or substituted as described earlier), R¹⁰ is piperazinyl ring and R^{a} is as previously described.

In another embodiment of Formula I, wherein X, Y, R², A, R¹⁰, R^{a} and the other moieties are independently selected, the moiety:

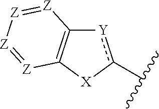

is:

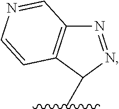

the moiety:

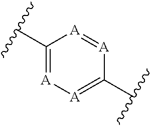

is

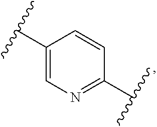

R² is piperazinyl (unsubstituted or substituted as described earlier), R¹⁰ is piperidinyl ring with —C(O)—O—R^{a}, and R³ is as previously described.

In another embodiment of Formula I, wherein X, Y, Z, R², A, R¹⁰, R^{a} and the other moieties are independently selected, the moiety:

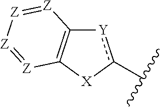

is:

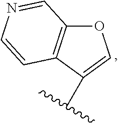

the moiety:

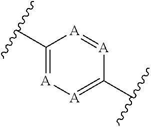

is

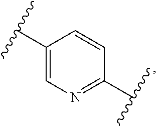

R² is morpholinyl (unsubstituted or substituted as described earlier), R¹⁰ is piperidinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

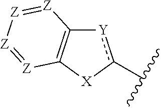

is:

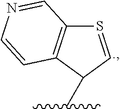

the moiety:

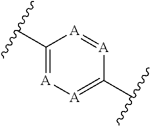

is

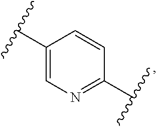

R² is pyrrolidinyl (unsubstituted or substituted as described earlier), R¹⁰ is piperazinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

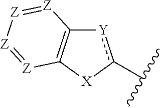

is:

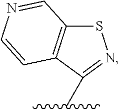

the moiety:

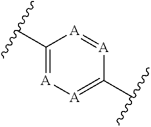

is

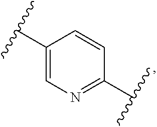

R² is piperidinyl (unsubstituted or substituted as described earlier), R¹⁰ is piperazinyl ring with —C(O)—O—R^{a}, and R^{a} is as previously described.

In another embodiment of Formula I, wherein X, Y, Z, R², A, R¹⁰, R^{a} and the other moieties are independently selected, the moiety:

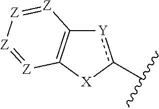

is:

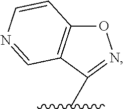

the moiety:

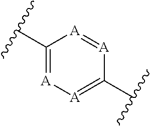

is

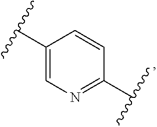

R² is piperazinyl (unsubstituted or substituted as described earlier), R¹⁰ is piperidinyl ring with —C(O)—O—R^{a}, and R^{a} is as previously described.

Non-limiting examples of the compounds of Formula I are shown in the Examples section. Several of the exemplified compounds exhibited IC₅₀ values less than 500 nM in the assay described later. Many compounds exhibited IC50 values less than 100 nM.

As used above, and throughout this disclosure, the following terms, unless otherwise indicated, shall be understood to have the following meanings:

“Patient” includes both humans and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. Lower alkyl means a group having about 1 to about 6 carbon atoms in the chain which may be straight or branched. Alkyl may be unsubstituted or optionally substituted by one or more substituents which may be the same or different, each substituent being independently selected from the group consisting of halo, alkyl, aryl, cycloalkyl, cyano, pyridine, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. Lower alkenyl means about 2 to about 6 carbon atoms in the chain which may be straight or branched. Alkenyl may be unsubstituted or optionally substituted by one or more substituents which may be the same or different, each substituent being independently selected from the group consisting of halo, alkyl. aryl, cycloalkyl, cyano, alkoxy and

—S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Lower alkynyl means about 2 to about 6 carbon atoms in the chain which may be straight or branched. Non-limiting examples of suitable alkynyl groups include ethynyl, propynyl, 2-butynyl and 3-methylbutynyl. Alkynyl may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridine (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

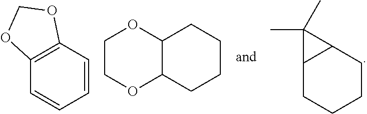

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic (e.g. bicyclic) ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, diazepinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such

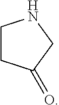

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

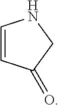

“Heterocyclenylalkyl” means a heterocyclenyl moiety as defined above linked via an alkyl moiety (defined above) to a parent core.

It should be noted that in heteroatom containing ring systems of this invention, there are no hydroxyl groups on carbon atoms adjacent to a N, O or S, as well as there are no N or S groups on carbon adjacent to another heteroatom. Thus, for example, in the ring:

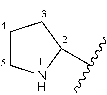

there is no —OH attached directly to carbons marked 2 and 5.

It should also be noted that tautomeric forms such as, for example, the moieties:

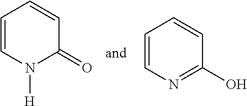

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkoxyalkyl-” means an alkyl-O-alkyl- group in which the alkyl group is as previously described. Non-limiting examples of suitable alkoxyalkyl groups include methoxymethyl, ethoxymethyl, n-propoxyethyl, isopropoxyethyl and n-butoxymethyl. The bond to the parent moiety is through the alkyl-.

“Aryloxy” means an aryl-O— group in which the aryl group is as previously described. Non-limiting examples of suitable aryloxy groups include phenoxy and naphthoxy. The bond to the parent moiety is through the ether oxygen.

“Aryloxyalkyl-” means an aryl-O-alkyl- group in which the aryl and aryl groups are as previously described. Non-limiting examples of suitable aryloxyalkyl groups include phenoxymethyl and naphthoxyethyl. The bond to the parent moiety is through the alkyl.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Alkylthioalkyl-” means an alkyl-5-alkyl- group in which the alkyl group is as previously described. Non-limiting examples of suitable alkylthioalkyl groups include methylthioethyl and ethylthiomethyl. The bond to the parent moiety is through the alkyl.

“Arylthio” means an aryl-S— group in which the aryl group is as previously described. Non-limiting examples of suitable arylthio groups include phenylthio and naphthylthio. The bond to the parent moiety is through the sulfur.

“Arylthioalkyl-” means an aryl-5-alkyl- group in which the aryl group is as previously described. Non-limiting examples of suitable arylthioalkyl groups include phenylthioethyl and phenylthiomethyl. The bond to the parent moiety is through the alkyl.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula I or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholine, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., (2004) 93(3), pp. 601-611 describe the preparation of the solvates of the antifungal fluconazole in ethyl acetate as well as from water. Similar preparations of solvates, hemisolvate, hydrates and the like are described by E. C. van Tonder et al, AAPS PharmSciTech., (2004) 5(1), article 12; and A. L. Bingham et al, Chem. Commun., (2001) pp. 603-604. A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “effective” or “therapeutically effective” is used herein, unless otherwise indicated, to describe an amount of a compound or composition which, in context, is used to produce or effect an intended result or therapeutic effect as understood in the common knowledge of those skilled in the art.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) pp. 1-19; P. Gould, International J. of Pharmaceutics (1986) (2001) 33 pp. 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula I may contain asymmetric or chiral centers, and, therefore, exist in different stereoisomeric forms. It is intended that all stereoisomeric forms of the compounds of Formula I as well as mixtures thereof, including racemic mixtures, form part of the present invention. In addition, the present invention embraces all geometric and positional isomers. For example, if a compound of Formula I incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula I may be atropisomers (e.g., substituted biaryls) and are considered as part of this invention. Enantiomers can also be separated by use of chiral HPLC column.

It is also possible that the compounds of Formula I may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula I incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention have pharmacological properties. The compounds of Formula I are inhibitors of DGAT, particularly DGAT1, and can be useful for the therapeutic and/or prophylactic treatment of diseases that are modulated by DGAT, particularly by DGAT1, such as, for example, metabolic syndrome, diabetes (e.g., Type 2 diabetes mellitus), obesity and the like.

The invention also includes methods of treating diseases that are modulated by DGAT, particularly by DGAT1.

The invention also includes methods of treating metabolic syndrome, diabetes (e.g., Type 2 diabetes mellitus), and obesity in a patient by administering at least one compound of Formula I to said patient.

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of the lipid, lipoprotein and apolipoprotein metabolism and other metabolic and hemodynamic disease. As such, the diabetic patient is at especially increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In Type 1 diabetes, or insulin-dependent diabetes mellitus (IDDM), patients produce little or no insulin, the hormone which regulates glucose utilization. In Type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for Type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a class of agents that can increase insulin sensitivity and bring about some degree of correction of hyperglycemia. However, the biguanides can induce lactic acidosis and nausea/diarrhea.

The glitazones (i.e. 5-benzylthiazolidine-2,4-diones) are a separate class of compounds with potential for the treatment of Type 2 diabetes. These agents increase insulin sensitivity in muscle, liver and adipose tissue in several animal models of Type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type 2 diabetes are agonists of the alpha, gamma or delta subtype, or a combination of these, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly Type 2 diabetes.

The invention includes compositions, e.g., pharmaceutical compositions, comprising at least one compound of Formula I. For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Other carriers include Poloxamer, Povidone K17, Povidone K12, Tween 80, ethanol, Cremophor/ethanol, polyethylene glycol (PEG) 400, propylene glycol, Trappsol, alpha-cyclodextrin or analogs thereof, beta-cyclodextrin or analogs thereof, or gamma-cyclodextrin or analogs thereof. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The therapeutic agents of the present invention are preferably formulated in pharmaceutical compositions and then, in accordance with the methods of the invention, administered to a subject, such as a human subject, in a variety of forms adapted to the chosen route of administration. For example, the therapeutic agents may be formulated for intravenous administration. The formulations may, however, include those suitable for oral, rectal, vaginal, topical, nasal, ophthalmic, or other parenteral administration (including subcutaneous, intramuscular, intrathecal, intraperitoneal and intratumoral, in addition to intravenous) administration.

Formulations suitable for parenteral administration conveniently include a sterile aqueous preparation of the active agent, or dispersions of sterile powders of the active agent, which are preferably isotonic with the blood of the recipient. Parenteral administration of the therapeutic agents (e.g., through an I.V. drip) is an additional form of administration. Isotonic agents that can be included in the liquid preparation include sugars, buffers, and sodium chloride. Solutions of the active agents can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the first and/or second therapeutic agents, or as a solution or suspension in an aqueous liquor or non-aqueous liquid such as a syrup, an elixir, an emulsion, or a draught. Such compositions and preparations may contain at least about 0.1 wt-% of the active agent. The amounts of the therapeutic agents should be such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

Preferably the compound is administered orally, intraperitoneally, or intravenously or intrathecally or some suitable combination(s) thereof.

Methods of administering small molecule therapeutic agents are well-known in the art.

The therapeutic agents described in the present disclosure can be administered to a subject alone or together (coadministered, optionally but not necessarily, in a single formulation) with other active agents as described herein, and are preferably administered with a pharmaceutically acceptable buffer. The therapeutic agents can be combined with a variety of physiological acceptable carriers, additives for delivery to a subject, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the therapeutic agent (i.e., the active agent) into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering the therapeutic agents to a subject in an amount effective to produce the desired effect. The therapeutic agents can be administered as a single dose or in multiple doses. Useful dosages of the active agents can be determined by comparing their in vitro activity and the in vivo activity in animal models.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Another aspect of the invention includes pharmaceutical compositions comprising at least one compound of Formula I and at least one other therapeutic agent in combination. Non-limiting examples of such combination agents are described below. The agents in the combination can be administered together as a joint administration (e.g., joint single pill), separately, one after the other in any order and the like as is well known in the art.

In the combination therapies of the present invention, an effective amount can refer to each individual agent or to the combination as a whole, wherein the amounts of all agents administered are together effective, but wherein the component agent of the combination may not be present individually in an effective amount.

### Combination Therapy

Accordingly, in one embodiment, the present invention provides methods for treating a Condition in a patient, the method comprising administering to the patient one or more Compounds of Formula I, or a pharmaceutically acceptable salt or solvate thereof and at least one additional therapeutic agent that is not a Compound of Formula I, wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during a time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer (such as a PPAR agonist, a DPP-IV inhibitor, a PTP-1B inhibitor and a glucokinase activator); a glucosidase inhibitor; an insulin secretagogue; a hepatic glucose output lowering agent; an anti-obesity agent; a meglitinide; an agent that slows or blocks the breakdown of starches and sugars in vivo; an histamine H₃ receptor antagonist; a sodium glucose uptake transporter 2 (SGLT-2) inhibitor; a peptide that increases insulin production; and insulin or any insulin-containing composition.

In one embodiment, the antidiabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

Non-limiting examples of insulin sensitizers include PPAR activators, such as rosiglitazone, pioglitazone and englitazone; biguanidines such as metformin and phenformin; DPP-IV inhibitors; PTP-1B inhibitors; and α-glucokinase activators, such as miglitol, acarbose, and voglibose.

Non-limiting examples of DPP-IV inhibitors useful in the present methods include sitagliptin (Januvia™, Merck), saxagliptin, denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of hepatic glucose output lowering agents include Glucophage and Glucophage XR.

Non-limiting examples of histamine H₃ receptor antagonist agents include the following compound:

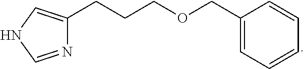

Non-limiting examples of insulin secretagogues include sulfonylurea and non-sulfonylurea drugs such as GLP-1, a GLP-1 mimetic, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exenatide, Liraglutide, CJC-1131 (ConjuChem, Exenatide-LAR (Amylin), BIM-51077 (Ipsen/LaRoche), ZP-10 (Zealand Pharmaceuticals), and compounds disclosed in International Publication No. WO 00/07617.

The term “insulin” as used herein, includes all pyridinones of insulin, including long acting and short acting forms of insulin.

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is an anti-obesity agent.

Non-limiting examples of anti-obesity agents useful in the present methods for treating diabetes include a 5-HT2C agonist, such as lorcaserin; a neuropeptide X antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

Non-limiting examples of meglitinides useful in the present methods for treating diabetes include repaglinide and nateglinide.

Non-limiting examples of insulin sensitizing agents include biguanides, such as metformin, metformin hydrochloride (such as GLUCOPHAGE® from Bristol-Myers Squibb), metformin hydrochloride with glyburide (such as GLUCOVANCE™ from Bristol-Myers Squibb) and buformin; glitazones; and thiazolidinediones, such as rosiglitazone, rosiglitazone maleate (AVANDIA™ from GlaxoSmithKline), pioglitazone, pioglitazone hydrochloride (ACTOS™, from Takeda) ciglitazone and MCC-555 (Mitsubishi Chemical Co.)

In one embodiment, the insulin sensitizer is a thiazolidinedione.

In another embodiment, the insulin sensitizer is a biguanide.

In another embodiment, the insulin sensitizer is a DPP-IV inhibitor.

In a further embodiment, the antidiabetic agent is a SGLT-2 inhibitor.

Non-limiting examples of antidiabetic agents that slow or block the breakdown of starches and sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g one is a tablet and one is a capsule. A kit comprising the separate dosage forms is therefore advantageous.

Generally, a total daily dosage of the one or more Compounds of Formula (I) and the additional therapeutic agent(s) can, when administered as combination therapy, range from about 0.1 to about 2000 mg per day, although variations will necessarily occur depending on the target of the therapy, the patient and the route of administration. In one embodiment, the dosage is from about 0.2 to about 1000 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about Ito about 100 mg/day, administered in a single dose or in 2-4 divided doses. In yet another embodiment, the dosage is from about 1 to about 50 mg/day, administered in a single dose or in 2-4 divided doses. In a farther embodiment, the dosage is from about 1 to about 20 mg/day, administered in a single dose or in 2-4 divided doses.

The compounds of the invention can be made according to the processes described below. The compounds of this invention are also exemplified in the examples below, which examples should not be construed as limiting the scope of the disclosure. Alternative mechanistic pathways and analogous structures within the scope of the invention may be apparent to those skilled in the art.

### General Methods

The general methods described in this paragraph were used unless stated otherwise in the examples below. All solvents and reagents were used as received. Proton NMR spectra were obtained using a Varian XL-400 (400 MHz) or a Bruker (500 MHz) instrument and were reported as parts per million (ppm) downfield from Me₄Si. LCMS analysis was performed using a PE SCIEX API-150EX, single quadrupole mass spectrometer equipped with a Phenomenex column: Gemini C-18, 50×4.6 mm, 5 micron; mobile phase A: 0.05% trifluoroacetic acid in water, B: 0.05% trifluoroacetic acid in CH₃CN; gradient: 90% A and 10% B to 5% A and 95% B in 5 minutes. Flash column chromatography was performed using Teledyne Isco RediSep Normal Phase Columns. Preparative TLC was performed using Analtech Silica gel GF plates.

### Intermediate A-3

### 3-methyl-5-phenylbenzofuran-2-carboxylic acid (A-3)

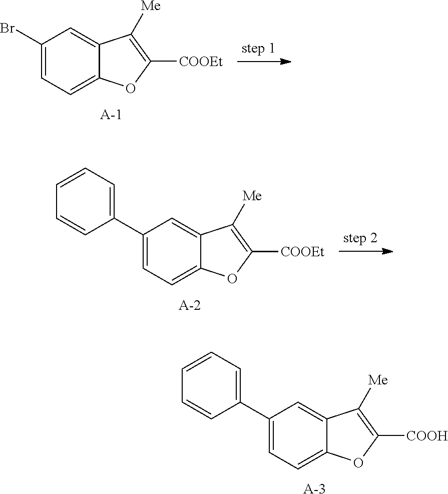

### Step 1: ethyl 3-methyl-5-phenylbenzofuran-2-carboxylate (A-2)

To a solution of compound A-1 (1.00 g, 3.53 mmol) dissolved in 1,2-dimethoxyethane (11 mL) was added tetrakis(triphenylphosphine)palladium (122 mg, 0.106 mmol). The mixture was stirred at room temperature for 10 mins then phenylboronic acid (0.470 g, 3.85 mmol) and sodium carbonate (8.5 mL, 1.0 M in water, 8.50 mmol) were added. The reaction mixture was heated at reflux for 4 h then cooled. Water was added, and the aqueous solution was extracted with CH₂Cl₂. The combined organic extract was dried (MgSO₄), filtered, and concentrated. Purification by silica gel chromatography (eluant: CH₂Cl₂) gave the product A-2 (0.80 g, 81% yield) as a white solid. MS (M+1): 281.2

### Step 2: 3-methyl-5-phenylbenzofuran-2-carboxylic acid (A-3)

To compound A-2 (0.91 g, 3.25 mmol) dissolved in THF (25 mL) and water (5 mL) was added lithium hydroxide monohydrate (0.41 g, 9.77 mmol). The reaction mixture was stirred at room temperature for 16 h then concentrated. 1 N HCl was added to acidify to pH=2. The white solid was isolated by filtration, washed with water then hexane, and dried to give the product A-3 (0.80 g, 98% yield) as a white solid. MS (M+1): 253.2

### Intermediate A-4

### 5-(2-fluorophenyl)-3-methylbenzofuran-2-carboxylic acid (A-4)

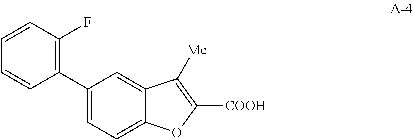

Intermediate A-4 was prepared by the general procedure for intermediate A-3. MS (M+1): 271

### Intermediate A-5

### 5-(3-fluorophenyl)-3-methylbenzofuran-2-carboxylic acid (A-5)

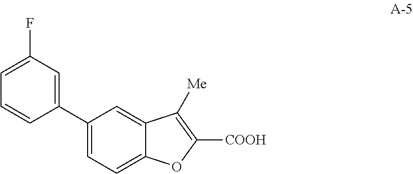

Intermediate A-5 was prepared by the general procedure for intermediate A-3. MS (M+1): 271

### Intermediate A-6

### 5-(4-fluorophenyl)-3-methylbenzofuran-2-carboxylic acid (A-6)

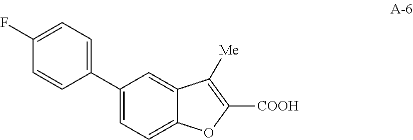

Intermediate A-6 was prepared by the general procedure for intermediate A-3. MS (M+1): 271

### Intermediate A-8

### 3-methyl-5-phenylbenzo[b]thiophene-2-carboxylic acid (A-8)

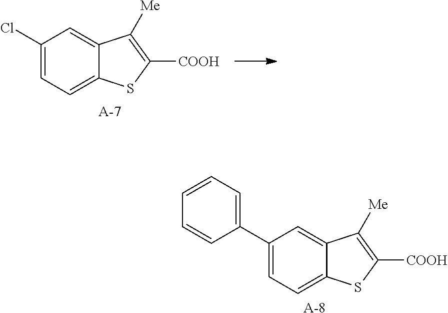

To compound A-7 (1.02 g, 4.50 mmol) in DMF (10 mL) and water (10 mL) under nitrogen was added phenyl boronic acid (0.66 g, 5.40 mmol), palladium diacetate (0.051 g, 0.225 mmol), sodium 2′-dicyclohexylphosphino-2,6-dimethoxy-1,1′biphenyl-3′-sulfonate hydrate (SPHOS-sulfonate, 0.23 g, 0.450 mmol), and potassium carbonate (1.87 g, 13.5 mmol). The mixture was heated at 80° C. for 6 h., 8.50 mmol) then cooled and concentrated. 0.5 N HCl (50 mL) was added, and the aqueous solution was extracted with 15% by volume EtOH in CH₂Cl₂. The combined organic extract was dried (MgSO₄), filtered, and concentrated. Purification by reverse phase chromatography (eluant: 10% CH₃CN in water with formic acid to 100% CH₃CN with formic acid) gave the product A-8 (0.56 g, 46% yield) as a white solid. MS (M+1): 269

### Intermediate A-9

### 3-methyl-5-(pyrimidin-5-yl)benzo[b]thiophene-2-carboxylic acid (A-9)

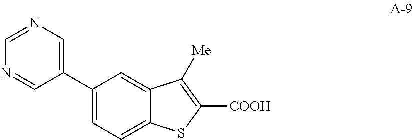

Intermediate A-9 was prepared by the general procedure for intermediate A-8. MS (M+1): 271

### Intermediate A-10

### 5-(4-fluorophenyl)-3-methylbenzofuran-2-carboxylic acid (A-10)

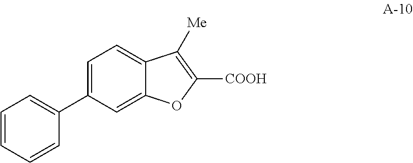

Intermediate A-10 was prepared by the general procedure for intermediate A-3. MS (M+1): 271

### Intermediate A-11

### 5-(3-fluorophenyl)-3-methylbenzo[b]thiophene-2-carboxylic acid (A-11)

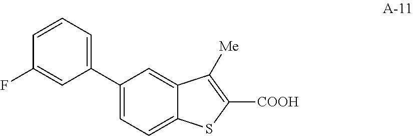

Intermediate A-11 was prepared by the general procedure for intermediate A-8. MS (M+Na): 309

### Intermediate A-12

### 5-(4-fluorophenyl)-3-methylbenzo[b]thiophene-2-carboxylic acid (A-12)

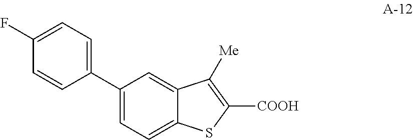

Intermediate A-12 was prepared by the general procedure for intermediate A-8. MS (M+Na): 309

### Intermediate A-16

### 3-(fluoromethyl)benzofuran-2-carboxylic acid (A-16)

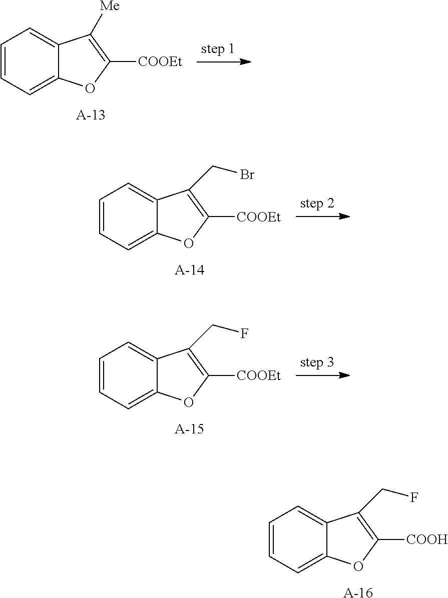

### Step 1: ethyl 3-(bromomethyl)benzofuran-2-carboxylate (A-14)

To compound A-13 (2.00 g, 9.79 mmol) dissolved in carbon tetrachloride (40 mL) was added N-bromosuccinimide (2.09 g, 11.75 mmol) and dibenzoyl peroxide (0.24 g, 0.979 mmol). The mixture was heated at reflux for 24 h then cooled. The solid was removed by filtration and washed with CH₂Cl₂. The filtrate was concentrated. Purification by silica gel chromatography (eluant: 5% to 10% EtOAc in hexane) gave the product A-14 (1.71 g, 62% yield) as a white solid. MS (M+1): 283

### Step 2: ethyl 3-(fluoromethyl)benzofuran-2-carboxylate (A-15)

To compound A-14 (0.50 g, 1.77 mmol) dissolved in dioxane (10 mL) was added pyridine (0.28 g, 0.29 mL, 3.53 mmol) and tetrabutylammonium hydrogen difluoride (50% in CH₃CN, 0.99 g, 2.3 mL, 153 mmol). The mixture was heated at 80° C. for 5 h then cooled and concentrated. Saturated NaHCO₃ (30 mL) was added, and the aqueous solution was extracted with CH₂Cl₂. The combined organic extract was dried (MgSO₄), filtered, and concentrated. Purification by silica gel chromatography (eluant: CH₂Cl₂) gave the product A-15 (0.20 g, 51% yield) as a colorless oil. MS (M+Na): 245

### Step 3: 3-(fluoromethyl)benzofuran-2-carboxylic acid (A-16)

Intermediate A-16 was prepared by the general procedure for intermediate A-3. MS (M+Na): 217

### Intermediate A-16

### 3-(difluoromethyl)benzofuran-2-carboxylic acid (A-19)

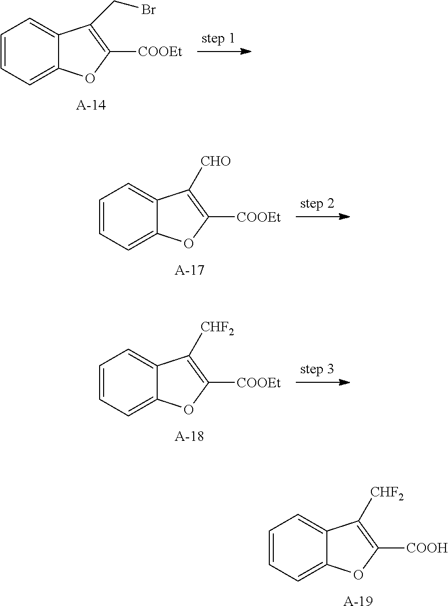

### Step 1: ethyl 3-formylbenzofuran-2-carboxylate (A-17)

To compound A-14 (1.20 g, 4.24 mmol) dissolved in dry DMF (25 mL) was added trimethylamine N-oxide (1.27 g, 16.95 mmol). The mixture was stirred at room temperature for 6 h then concentrated. Water (40 mL) was added, and the aqueous solution was extracted with CH₂Cl₂. The combined organic extract was dried (MgSO₄), filtered, and concentrated. Purification by silica gel chromatography (eluant: CH₂Cl₂) gave the product A-17 (0.27 g 29% yield) as a yellow solid. MS (M+1): 219

### Step 2: ethyl 3-(difluoromethyl)benzofuran-2-carboxylate (A-18)

To compound A-17 (270 mg, 1.24 mmol) dissolved in CH₂Cl₂ (10 mL) and cooled to 0° C. was added diethylaminosulfur trifluoride (300 mg, 0.25 mL, 1.86 mmol). The mixture was warmed slowly to room temperature over 1.5 h then stirred at room temperature for 3.5 h. Saturated NaHCO₃ (25 mL) was added, and the aqueous solution was extracted with CH₂Cl₂. The combined organic extract was dried (MgSO₄), filtered, and concentrated. Purification by silica gel chromatography (eluant: 5% EtOAc—CH₂Cl₂) gave the product A-18 (150 mg, 51% yield) as a white solid. MS (M+1): 241

### Step 3: 3-(difluoromethyl)benzofuran-2-carboxylic acid (A-19)

Intermediate A-19 was prepared by the general procedure for intermediate A-3. MS (M+Na+1): 236

### Intermediate B-5

### 4-(5-Aminopyridin-2-yl)-N-(2-fluorophenyl)piperazine-1-carboxamide (B-5)

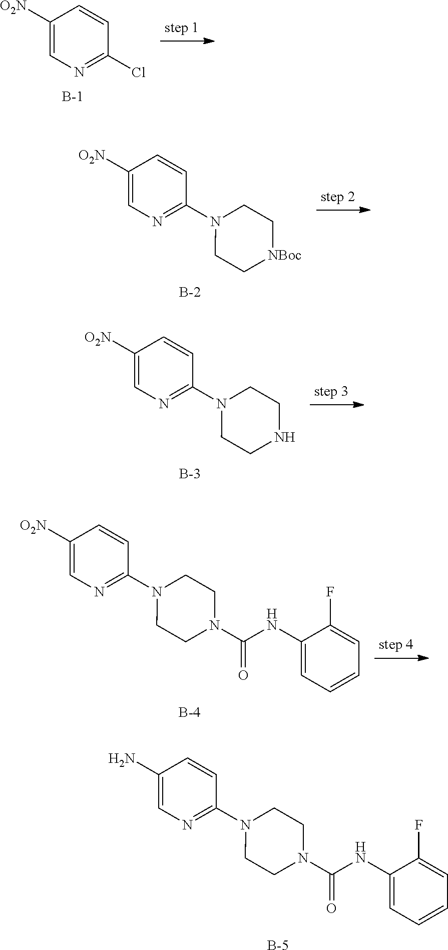

### Step 1: t-Butyl 4-(5-nitropyridin-2-yl)piperazine-1-carboxylate (B-2)

To 5-nitro-2-chloropyridine (10.0 g, 0.0631 mol) and N—BOC-piperazine (17.6 g, 0.0946 mol) dissolved in DMF (200 mL) was added N,N-diisopropylethylamine (24.5 g, 31.3 mL, 0.189 mol). The reaction mixture was heated at 100° C. for 16 h then cooled to RT and concentrated. Water (300 mL) was added, and the aqueous solution was extracted with CH₂Cl₂. The combined organic extract was dried (MgSO₄), filtered, and concentrated. Purification by vacuum filtration through silica gel (eluant: 5% EtOAc—CH₂Cl₂) gave t-butyl 4-(5-nitropyridin-2-yl)piperazine-1-carboxylate (B-2) as a yellow solid (19.45 g, 100% yield). MS (M+1): 309.

### Step 2: N-(5-nitropyridin-2-yl)piperazine (B-3)

To compound B-2 (19.45 g, 0.0631 mol) dissolved in CH₂Cl₂ (250 mL) and cooled to 0° C. was added trifluoroacetic acid (50 mL). The resulting reaction mixture was stirred at RT for 16 h then concentrated. The crude product was dissolved in CH₂Cl₂ (250 mL) and made basic with the addition of 1 N aqueous NaOH (200 mL) and 3 N aqueous NaOH (100 mL). The layers were separated, and the aqueous solution extracted with CH₂Cl₂ The combined organic extract was dried (MgSO₄), filtered, and concentrated to give the product N-(2-fluorophenyl)-4-(5-nitropyridin-2-yl)piperazine-1-carboxamide (B-3) as a yellow solid (13.13 g, 100% yield). MS (M+1): 209.

### Step 3: 4-(5-Nitropyridin-2-yl)-N-(2-fluorophenyl)piperazine-1-carboxamide (B-4)

To compound B-3 (6.6 g, 32 mmol) dissolved in dry THF (200 mL) was added triethylamine (8.8 mL, 63 mmol) and 2-fluorophenyl isocyanate (4.3 mL, 38 mmol). The resulting reaction mixture was heated at 80° C. for 16 h then cooled to RT and concentrated. Water (150 mL) was added, and the aqueous solution was extracted with CH₂Cl₂. The combined organic extract was dried (MgSO₄), filtered, and concentrated to give a yellow solid. The solid was triturated with water, filtered, and dried to give the product 4-(5-nitropyridin-2-yl)-N-(2-fluorophenyl)piperazine-1-carboxamide (B-4) as a yellow solid (11.4 g, 100% yield). MS (M+1): 346.

### Step 4: 4-(5-Aminopyridin-2-yl)-N-(2-fluorophenyl)piperazine-1-carboxamide (B-5)

To compound 13-4 (11.0 g, 31.8 mmol) suspended in ethyl acetate (100 mL) and isopropanol (100 mL) under a nitrogen atmosphere was added platinum dioxide catalyst (0.72 g, 3.18 mmol). The resulting reaction mixture was stirred at RT under a hydrogen atmosphere (balloon) for 16 h. The catalyst was removed by filtration through celite and washed with isopropanol. The filtrate was concentrated to give the product 4-(5-aminopyridin-2-yl)-N-(2-fluorophenyl)piperazine-1-carboxamide (B-5) as a white solid (9.2 g, 92% yield). MS (M+1): 316.

### Intermediate B-6

### ethyl 2-(4-(5-aminopyridin-2-yl)piperazine-1-carboxamido)benzoate (B-6)

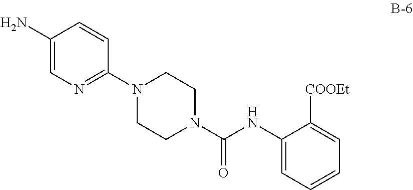

Intermediate B-6 was prepared by the general procedure for intermediate B-5. MS (M+1): 370.

### Intermediate B-9

### methyl 3-((1-(5-aminopyridin-2-yl)piperidin-4-yloxy)methyl)benzoate (N-9)

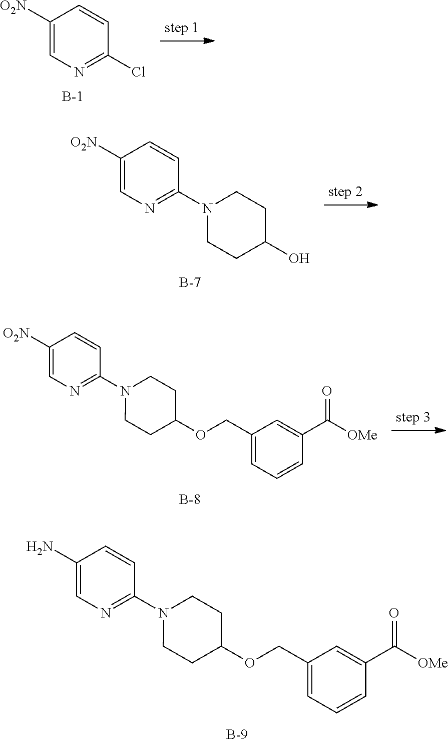

### Step 1: 1-(5-nitropyridin-2-yl)piperidin-4-ol (B-7)

Compound 13-7 was prepared by the general procedure for compound 13-2. MS (M+1): 224

### Step 2: methyl 34(1-(5-nitropyridin-2-yl)piperidin-4-yloxy)methyl)benzoate (B-8)

To compound B-7 (1.00 g, 4.48 mmol) in dry THF (20 mL) under nitrogen was added sodium hydride (0.215 g of 60 wt % in oil, 5.38 mmol). The mixture was stirred at room temperature for 20 mins then added 3-(bromomethyl)benzoate (1.54 g, 6.72 mmol) and tetrabutylammmonium iodide (0.41 g, 1.12 mmol). The reaction mixture was heated at reflux for 4 h then cooled and concentrated. Water (50 mL) was added, and the aqueous solution was extracted with, CH₂Cl₂. The combined extracts were dried (MgSO₄), filtered, and concentrated. Purification by silica gel chromatography (eluant: CH₂Cl₂ to 6% EtOAc—CH₂Cl₂) gave the product 8-8 (0.96 g, 58% yield) as a yellow oil. MS (M+1): 372

### Step 3: methyl 3-((1-(5-aminopyridin-2-yl)piperidin-4-yloxy)methyl)benzoate (B-9)

Compound B-9 was prepared by the general procedure for compound 8-5. MS (M+1): 342.5

### Intermediate B-13

### methyl 3-((1-(5-aminopyridin-2-yl)piperidin-4-yl)methoxy)benzoate (B-13)

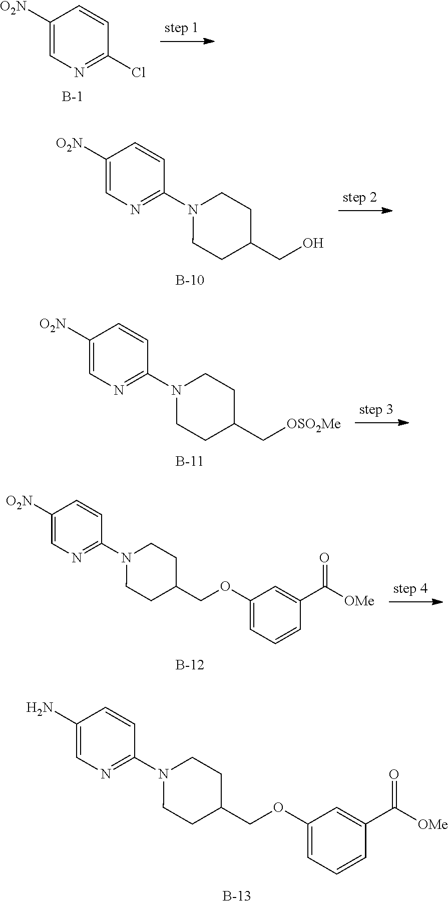

### Step 1: (1-(5-nitropyridin-2-yl)piperidin-4-yl)methanol (B-10)

Compound B-10 was prepared by the general procedure for compound B-2. MS (M+1): 238

### Step 2: (1-(5-nitropyridin-2-yl)piperidin-4-yl)methyl methanesulfonate (B-11)

To compound 8-10 (1.00 g, 4.21 mmol) in CH₂Cl₂ (30 mL) and cooled to 0° C. was added triethylamine (0.85 g, 1.2 mL, 8.43 mmol) and mesyl chloride (0.60 g, 0.41 mL, 5.27 mmol). The reaction mixture was stirred at 0° C. for 15 mins then at room temperature for 60 mins. Water (50 mL) was added, and the aqueous solution was extracted with CH₂Cl₂. The combined extracts were dried (MgSO₄), filtered, and concentrated to give the product B-11 (1.33 g, 100% yield). MS (M+1): 316

### Step 3: methyl 3-((1-(5-nitropyridin-2-yl)piperidin-4-yl)methoxy)benzoate (B-12)

To methyl 3-hydroxybenzoate (0.96 g, 6.28 mmol) in dry DMF (20 mL) under nitrogen was added sodium hydride (0.25 g of 60 wt % in oil, 6.28 mmol). The mixture was stirred at room temperature for 15 mins then added compound B-11 (1.32 g, 4.19 mmol) in dry DMF (10 L). The resulting mixture was heated at 50° C. for 5 h then cooled and concentrated. Water (50 mL) was added, and the aqueous solution was extracted with CH₂Cl₂. The combined extracts were dried (MgSO₄), filtered, and concentrated. Purification by silica gel chromatography (eluant: 5% EtOAc—CH₂Cl₂) gave the product B-12 (1.22 g, 78% yield). MS (M+1): 372

### Step 4: methyl 3-((1-(5-aminopyridin-2-yl)piperidin-4-yl)methoxy)benzoate (B-13)

Compound B-13 was prepared by the general procedure for compound B-5. MS (M+1): 342

### INTERMEDIATE B-19

### methyl 3-(5-(4-aminophenyl)pyridin-2-yloxy)-2,2-dimethylpropanoate (B-19)

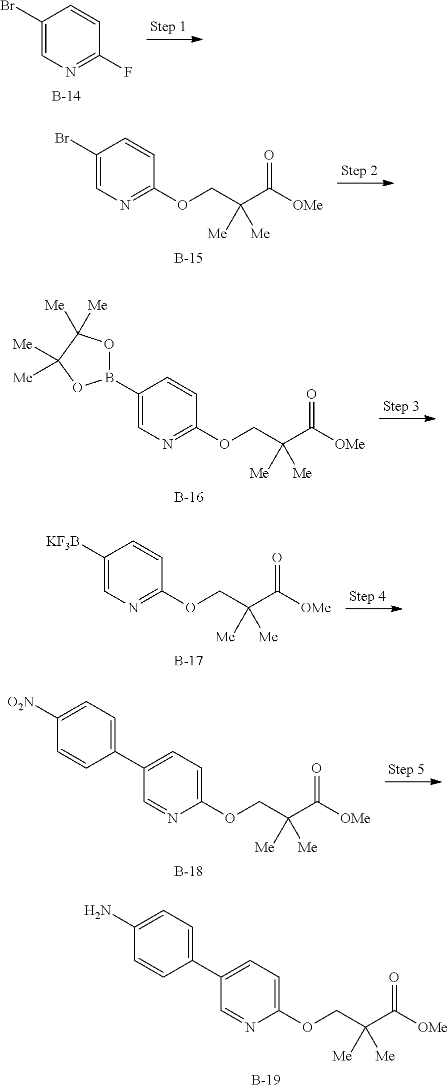

### Step 1: methyl 3-(5-bromopyridin-2-yloxy)-2,2-dimethylpropanoate (B-15)

Sodium hydride (60% disp. in oil, 908.0 mg, 22.7 mmol) was added portionwise at RT to a solution of 5-bromo-2-fluoropyridine B-14 (1.16 mL, 11.4 mmol) and methyl 3-hydroxy-2,2-dimethylpropanoate (1.88 mL, 14.8 mmol) in anhydrous THF (33 mL) and dry 1,3-dimethyl-3,4,5,6-tetrahydro-2(1H)-pyrimidinone (5.0 mL) under argon. After 2 h of stirring at RT, the reaction mixture was heated at 50° C. for 10 h and then to 70° C. for 3 h. The suspension was cooled to RT, filtered over a thin celite pad, rinsed with Et₂O (150 mL). The filtrate was concentrated to a volume of ca. 5 mL, then diluted with Et₂O (150 mL), quenched with water (90 mL) and decanted. The aqueous layer was extracted with Et₂O; the combined organic extract was successively washed with water, brine, dried (MgSO₄), filtered and concentrated. Purification by silica gel chromatography (eluant: 0-20% EtOAc/hexanes) yielded the title compound B-15 as a clear oil (1.796 g, 55% yield). MS (M+1): 288, 290.

### Step 2: methyl 2,2-dimethyl-3-(5-(4,4,5,5-tetramethyl-1,3,2-dioxaborolan-2-yl)pyridin-2-yloxy)propanoate (B-16)

[1,1′-Bis(diphenylphosphino)ferrocene]dichloropalladium(II) (217 mg, 0.27 mmol), potassium acetate (1.57 g, 16.0 mmol), bis(pinacolato)diboron (1.62 g, 6.39 mmol) and methyl 3-(5-bromopyridin-2-yloxy)-2,2-dimethylpropanoate B-15 (1.53 g, 5.32 mmol) were mixed at RT in anhydrous 1,4-dioxane (26.6 mL). The reaction was degassed several times, placed under argon and stirred at 60° C. for 10 h. After cooling, the mixture was filtered through a celite pad, washed with EtOAc and concentrated. Purification by silica gel chromatography (eluant: 5-95% EtOAc/hexanes) yielded the title compound B-16 as a white solid (750 mg; 42% yield). MS (M+1): 3361

### Step 3: potassium trifluoro(6-(3-methoxy-2,2-dimethyl-3-oxopropoxy)pyridin-3-yl)borate (B-17)

Potassium hydrogen difluoride (525 mg, 6.72 mmol) was added at RT to a solution of methyl 2,2-dimethyl-3-(5-(4,4,5,5-tetramethyl-1,3,2-dioxaborolan-2-yl)pyridin-2-yloxy)propanoate B-16 (750 mg, 2.24 mmol) in a 2:1 mixture of water and methanol (11 mL). The reaction was stirred at RT in a polypropylene reactor for 4 h. The reaction mixture was then concentrated, the resulting residue was triturated in ice-cold water (50 mL), quickly filtered, washed with ice-cold diethyl ether (250 mL) and dried under high vacuum to give title product B-17 as a white solid (700 mg, 98% yield).

### Step 4: methyl 2,2-dimethyl-3-(5-(4-nitrophenyl)pyridin-2-yloxy)propanoate (B-18)

Intermediate 8-17 potassium trifluoro(6-(3-methoxy-2,2-dimethyl-3-oxopropoxy)pyridin-3-yl)borate (175 mg, 0.55 mmol), 1-bromo-4-nitrobenzene (93.3 mg, 0.46 mmol), anhydrous potassium carbonate (160 mg, 1.16 mmol) and [1,3-bis(2,6-diisopropylphenyl)imidazol-2-ylidene](3-chloropyridyl)palladium(II) dichloride (PEPPSI-iPr, 31 mg, 0.046 mmol) were mixed in 1:1 EtOH:water (4.62 mL) in a 5 mL microwave reactor vial, evacuated several times and placed under argon. The reaction mixture was sealed, heated at 60° C. for 30 min under microwave irradiation, then cooled to RT and concentrated. Purification by silica gel chromatography (eluant: 5-100% EtOAc/hexanes) yielded the title compound B-18 as a light beige solid (122 mg; 80% yield). MS (M+1): 331.2.

### Step 5: methyl 3-(5-(4-aminophenyl)pyridin-2-yloxy)-2,2-dimethylpropanoate (8-19)

To methyl 2,2-dimethyl-3-(5-(4-nitrophenyl)pyridin-2-yloxy)propanoate B-18 (122 mg, 0.37 mmol) dissolved in EtOAc (1.65 mL) and EtOH (1.65 mL) under a nitrogen atmosphere was added platinum dioxide (20 mg). The resulting reaction mixture was stirred at RT under a hydrogen atmosphere (balloon) for 16 h. The catalyst was removed by filtration through celite and washed with EtOH. The filtrate was concentrated to yield Intermediate B-19 as a light beige solid (113 mg, 94% yield). MS (M+1): 301.2.

### INTERMEDIATE B-25

### methyl 3-((1-(5-aminopyrimidin-2-yl)piperidin-4-yl)methoxy)benzoate (B-25)

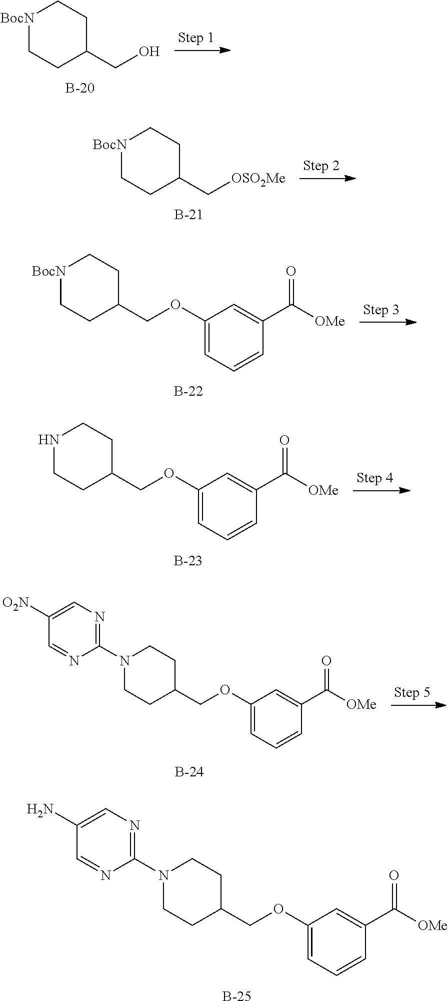

### Step 1: tert-butyl 4-((methylsulfonyloxy)methyl)piperidine-1-carboxylate (B-21)

To tert-butyl 4-(hydroxymethyl)piperidine-1-carboxylate B-20 (3.8 g, 17.4 mmol) in CH₂Cl₂ (70 mL) at 0° C. was added N,N-diisopropylethylamine (6.1 mL, 34.8 mmol) and mesyl chloride (2.0 mL, 26.1 mmol). The reaction mixture was stirred at 0° C. for 20 min then at RT for 1 h. Water (50 mL) was added, and the aqueous solution was extracted with Et₂O. The combined extract was successively washed with water then brine, dried (MgSO₄), filtered, and concentrated to yield the title compound B-21 as an orange oil (5.15 g, 100% yield).

### Step 2: tert-butyl 4-((3-(methoxycarbonyl)phenoxy)methyl)piperidine-1-carboxylate (B-22)

To methyl 3-hydroxybenzoate (3.97 g, 26.10 mmol) in dry DMF (50 mL) under argon was added sodium hydride (1.18 g of 60 wt % in oil, 29.6 mmol). The mixture was stirred at RT for 15 min then added tert-butyl 4-((methylsulfonyloxy)methyl)piperidine-1-carboxylate B-21 (5.15 g, 17.4 mmol) in dry DMF (35 mL). The resulting mixture was heated at 50° C. for 16 h then cooled and concentrated. Water (50 mL) was added, and the aqueous solution was extracted with Et₂O. The combined extracts was successively washed with water then brine, dried (MgSO₄), filtered, and concentrated. Purification by silica gel chromatography (eluant: 5-95% EtOAc/hexanes) yielded the title compound B-22 as a solid (4.10 g, 67% yield). MS (M+Na): 372.2.

### Step 3: methyl 3-(piperidin-4-ylmethoxy)benzoate (B-23)

tert-Butyl 4-((3-(methoxycarbonyl)phenoxy)methyl)piperidine-1-carboxylate B-22 was stirred at RT in 4N HCl in dioxane for 3 h. The reaction mixture was then concentrated, and the solid residue was triturated in Et₂O (100 mL), filtered and rinsed with Et₂O. The residue was then dissolved in EtOAc, then successively washed with Na₂CO₃ (sat) then brine, dried (MgSO₄), filtered, and concentrated to give intermediate B-23 as a light tan solid (2.60 g, 88% yield). MS (M+1): 250.1.

### Step 4: methyl 3-((1-(5-nitropyrimidin-2-yl)piperidin-4-yl)methoxy)benzoate (B-24)

Methyl 3-(piperidin-4-ylmethoxy)benzoate B-23 (507 mg, 2.04 mmol), 2-chloro-5-nitropyrimidine (250 mg, 1.57 mmol) and N,N-diisopropylethylamine (0.55 mL, 3.13 mmol) were dissolved in ααα-trifluorotoluene (3.90 mL) under nitrogen in a microwave reactor vial. The reaction mixture was sealed and heated under microwave irradiation for 120 min at 120° C. EtOAc and water were added and the mixture was decanted. The aqueous solution was extracted with EtOAc, and the combined organic extract was successively washed with water then brine, dried (MgSO₄), filtered, and concentrated. Purification by silica gel chromatography (eluant: 10-100% EtOAc/hexanes) yielded the title compound B-24 as a solid (159 mg, 27% yield). MS (M+1): 373.2.

### Step 5: methyl 3-((1-(5-aminopyrimidin-2-yl)piperidin-4-yl)methoxy)benzoate (B-25)

To methyl 3-((1-(5-nitropyrimidin-2-yl)piperidin-4-yl)methoxy)benzoate B-24 (159 mg, 0.42 mmol) dissolved in EtOAc (1.05 mL) and EtOH (1.05 mL) under a nitrogen atmosphere was added platinum dioxide (35 mg). The resulting reaction mixture was stirred at RT under a hydrogen atmosphere (balloon) for 20 h. The catalyst was removed by filtration through celite and washed with EtOH. The filtrate was concentrated to yield crude Intermediate B-25 as a pale yellow solid (110 mg, ca. 68% yield). MS (M+1): 343.2.

### INTERMEDIATE B-27

### methyl 3-((1-(4-aminophenyl)piperidin-4-yl)methoxy)benzoate (B-27)

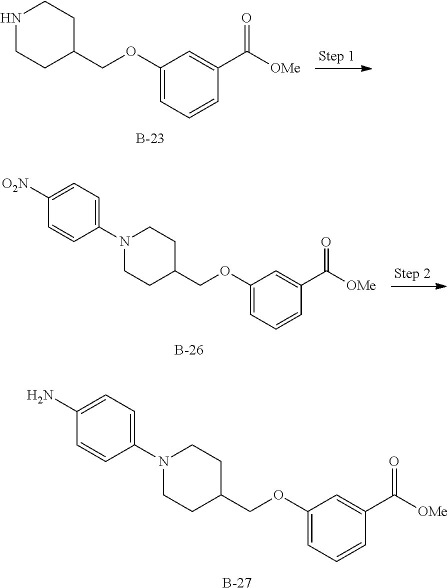

### Step 1: methyl 3-((1-(4-nitrophenyl)piperidin-4-yl)methoxy)benzoate (B-26)

A solution of methyl 3-(piperidin-4-ylmethoxy)benzoate B-23 (288 mg, 1.16 mmol) in acetonitrile (3.0 mL) was added at RT to a solution of 1-fluoro-4-nitrobenzene (0.123 mL, 1.16 mmol) and N,N-diisopropylethylamine (0.40 mL, 2.31 mmol) in dry acetonitrile (2.8 mL) under nitrogen. The reaction mixture was refluxed for 9 h at 85° C. and then concentrated to dryness. The residue was dissolved in EtOAc and water, and the mixture was decanted. The aqueous solution was extracted with EtOAc, and the combined organic extract was successively washed with water then brine, dried (MgSO₄), filtered, and concentrated. Purification by silica gel chromatography (eluant: 10-100% EtOAc/hexanes) yielded the title compound 8-26 as a yellow solid (295.0 mg, 69% yield). MS (M+1): 371.2.

### Step 2: methyl 341-(4-aminophenyl)piperidin-4-yl)methoxy)benzoate (B-27)

To methyl 341-(4-nitrophenyl)piperidin-4-yl)methoxy)benzoate 8-26 (295.0 mg, 0.80 mmol) dissolved in EtOAc (1.96 mL) and EtOH (1.96 mL) under a nitrogen atmosphere was added platinum dioxide (80.0 mg). The resulting reaction mixture was stirred at RT under a hydrogen atmosphere (balloon) for 24 h. The catalyst was removed by filtration through celite and washed with EtOH. The filtrate was concentrated to yield crude Intermediate B-27 as a beige solid (273 mg, ca. 95% yield). MS (M+1): 341.3.

### Intermediate B-28

### methyl 3-(5-amino-2,3′-bipyridin-6′-yloxy)-2,2-dimethylpropanoate (B-28)

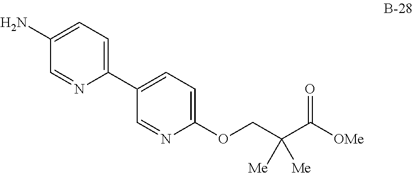

Intermediate B-28 was prepared by the general procedure for intermediate B-19. MS (M+1): 302.2.

### Intermediate B-29

### methyl 3-((1-(4-amino-2-fluorophenyl)piperidin-4-yl)methoxy)benzoate (B-29)

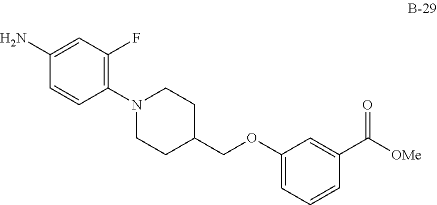

Intermediate B-29 was prepared by the general procedure for intermediate B-27. MS (M+1): 359.2.

### INTERMEDIATE B-33

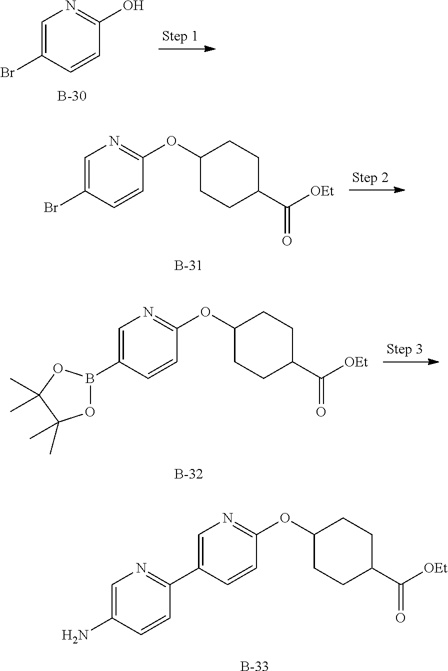

### Step 1: ethyl 4-(5-bromopyridin-2-yloxy)cyclohexanecarboxylate (B-31)

To a solution of 5-bromo-2-hydroxypyridine B-30 (1.0 g, 5.75 mmol) and ethyl 4-hydroxycyclohexanecarboxylate (990 mg, 5.75 mmol) in THF (50 mL) was added PPh₃ (2.4 g, 9.150 mmol) and DIAD (1.8 mL, 9.14 mmol). The reaction mixture was stirred at RT for 17 h. The solution was then concentrated and purified by ISCO to yield ethyl 4-(5-bromopyridin-2-yloxy)cyclohexanecarboxylate as a colorless oil (1.16 g, 62% yield, 3:2 trans:cis ratio).

### Step 2: ethyl 4-(5-(4,4,5,5-tetramethyl-1,3,2-dioxaborolan-2-yl)pyridin-2-yloxy)cyclohexanecarboxylate (B-32)

To a solution of ethyl 4-(5-bromopyridin-2-yloxy)cyclohexane-carboxylate (1.23 g, 4.84 mmol) and bis(pinacolato)diboron (1.16 g, 3.53 mmol) in 1,4 dioxane (30 mL) was added KOAc (1.98 g, 20.17 mmol) and Pd(dppf)Cl₂*CH₂Cl₂ (164 mg, 0.201 mmol). The reaction mixture was heated to reflux and stirred for 15 h. The solution was filtered through celite, concentrated and purified by ISCO to yield ethyl 4-(5-(4,4,5,5-tetramethyl-1,3,2-dioxaborolan-2-yl)pyridin-2-yloxy)cyclohexanecarboxylate as a colorless oil (1.18 g, 89% yield 1:1 trans:cis ratio).

### Step 3: ethyl 4-(5-amino-2,3′-bipyridin-6′-yloxy)cyclohexanecarboxylate (B-33)

To a solution of ethyl 4-(5-(4,4,5,5-tetramethyl-1,3,2-dioxaborolan-2-yl)pyridin-2-yloxy)cyclohexanecarboxylate (300 mg, 0.799 mmol) and 5-amino-2-bromopyridine (160 mg, 0.924 mmol) in 1,4 dioxane:H₂O (3:1, 20 mL) was added tetrakis(triphenylphosphine)palladium(0) (194 mg, 0.168 mmol) and Na₂CO₃ (267 mg, 2.52 mmol). The reaction mixture was heated to reflux and stirred for 15 h. The solution was filtered through celite, concentrated and purified by ISCO to yield ethyl 4-(5-amino-2,3′-bipyridin-6′-yloxy)cyclohexanecarboxylate as a brown oil (191 mg, 70% yield 1:1 trans:cis ratio). MS (M+1): 342.

### Example 1

### N-(2-fluorophenyl)-4-(5-(3-methylbenzofuran-2-carboxamido)pyridin-2-yl)piperazine-1-carboxamide (1)

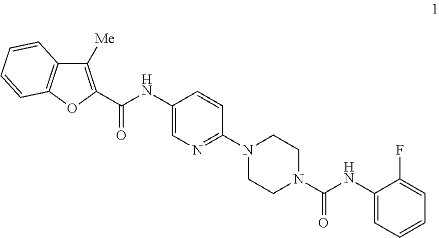

To 3-methylcoumarilic acid (100 mg, 0.57 mmol) in dry DMF (7.5 mL) was added amine B-5 (233 mg, 0.740 mmol), HATU (432 mg, 1.14 mmol), and Hunig's base (0.20 mL, 1.14 mmol). The reaction mixture was stirred at room temperature for 16 h then concentrated. Water was added, and the aqueous solution was extracted with CH₂Cl₂. The combined organic extract was dried (MgSO4), filtered, and concentrated. Purification by silica gel chromatography (eluant: EtOAc) gave a solid which was triturated with CH₂Cl₂ to give the product 1 (149 mg, 90% yield) as a light yellow solid.

¹H NMR (500 MHz, DMSO-d6) δ 10.42 (s, 1H), 8.56 (s, 1H), 8.42 (s, 1H), 8.04 (d, 1H, J-8.5 Hz), 7.80 (d, 8 Hz), 7.67 (d, 1H, J=8.5 Hz), 7.53 (t, 1H, J=8.5 Hz), 7.45 (m, 1H), 7.39 (t, 1H, J=7 Hz), 7.20 (m, 1H), 7.13 (m, 2H), 6.99 (d, 1H, J=9 Hz), 3.58 (broad s, 4H), 3.56 (broad s, 4H), 2.60 (s, 3H). MS (M+1): 474.2

### Example 2

### (R)—N-(6-(4-(2-hydroxy-2-phenylacetyl)piperazin-1-yl)pyridin-3-yl)-3-methylbenzofuran-2-carboxamide (2)

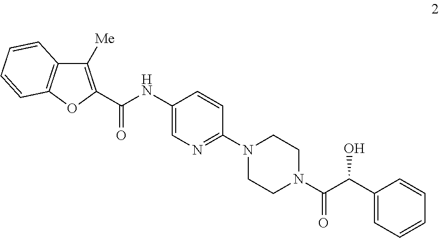

Compound 2 was prepared by the general procedure for compound 1.

¹H NMR (500 MHz, DMSO-d6) δ 10.37 (s, 1H), 8.51 (d, 1H, J=2 Hz), 7.97 (d, 1H, J=9.5, 2.5 Hz), 7.79 (d, 1H, J=7.5 Hz), 7.66 (d, 1H, J=8.5 Hz), 7.52 (t, 1H, J=7 Hz), 7.40 (m, 5H), 7.30 (t, 1H, J=7 Hz), 6.85 (d, 1H, J=9 Hz), 5.47 (s, 1H), 3.61 (m, 3H), 3.45 (m, 2H), 3.40 (m, 1H), 3.25 (m, 1H), 3.09 (m, 1H), 2.59 (s, 3H). MS (M+1): 471.2

### Example 3

### 3-methyl-N-(6-(4-(3,3,3-trifluoro-2-hydroxypropanoyl)piperazin-1-yl)pyridin-3-yl)benzofuran-2-carboxamide (3)

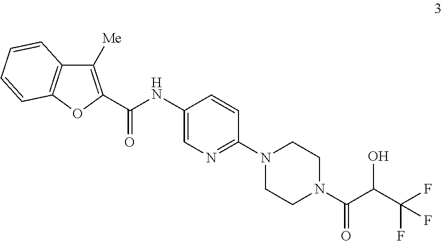

Compound 3 was prepared by the general procedure for compound 1.

¹H NMR (500 MHz, DMSO-d6) δ 10.40 (s, 1H), 8.55 (d, 1H, J=2.5 Hz), 8.02 (d, 1H, J=9, 2.5 Hz), 7.80 (d, 1H, J=8 Hz), 7.67 (d, 1H, J=8 Hz), 7.53 (t, 1H, J=8.5 Hz), 7.39 (t, 1H, J=7.5 Hz), 6.95 (d, 1H, J=9 Hz), 6.80 (broad s, 1H), 5.19 (broad s, 1H), 3.68 (m, 3H), 3.56 (m, 3H), 3.48 (m, 2H), 2.60 (s, 3H). MS (M+1): 463.2

### Example 4

### N-(6-(4-(2-(2,6-difluorophenyl)acetyl)piperazin-1-yl)pyridin-3-yl)-3-methylbenzofuran-2-carboxamide (4)

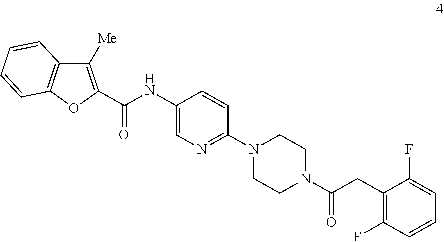

Compound 4 was prepared by the general procedure for compound 1.

¹H NMR (500 MHz, DMSO-d6) δ 10.39 (s, 1H), 8.56 (d, 1H, J=3 Hz), 8.01 (d, 1H, J=9, 2.5 Hz), 7.80 (d, 1H, J=8 Hz), 7.67 (d, 1H, J=8 Hz), 7.53 (t, 1H, J=8 Hz), 7.39 (t, 2H, J=7.5 Hz), 7.08 (t, 2H, J=7.5 Hz), 6.92 (d, 1H, J=9 Hz), 3.83 (s, 2H), 3.75 (broad s, 2H), 3.59 (broad s, 4H), 3.49 (broad s, 2H), 2.60 (s, 3H). MS (M+1): 491.2

### Example 5

### 4-(5-(5-fluoro-3-methylbenzofuran-2-carboxamido)pyridin-2-yl)-N-(2-fluorophenyl)piperazine-1-carboxamide (5)

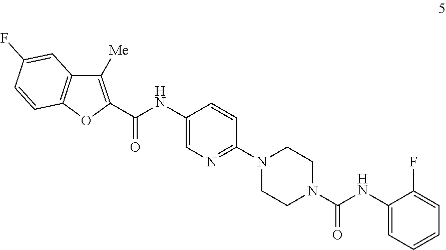

Compound 5 was prepared by the general procedure for compound 1 using intermediate B-5.

¹H NMR (500 MHz, DMSO-d6) δ 10.42 (s, 1H), 8.55 (d, 1H, J=3 Hz), 8.42 (s, 1H), 8.00 (dd, 1H, J=8.5, 3 Hz), 7.69 (dd, 1H, J=9, 4 Hz), 7.66 (dd, 1H, J=8.5, 2.5 Hz), 7.45 (m, 1H), 7.37 (td, 1H, J=9, 3 Hz), 7.20 (m, 1H), 7.13 (m, 2H), 6.93 (d, 1H, J=9.5 Hz), 3.57 (m, 4H), 3.53 (m, 4H), 2.57 (s, 3H). MS (M+1): 492.1

### Example 6

### N-(2-fluorophenyl)-4-(5-(3,6,7-trimethylbenzofuran-2-carboxamido)pyridin-2-yl)piperazine-1-carboxamide (6)

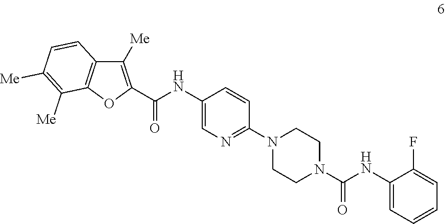

Compound 6 was prepared by the general procedure for compound 1 using intermediate B-5.

¹H NMR (500 MHz, DMSO-d6) δ 10.12 (s, 1H), 8.51 (s, 1H), 8.43 (s, 1H), 7.95 (d, 1H, J=9 Hz), 7.45 (d, 2H, J=7.5 Hz), 7.20 (m, 1H), 7.17 (d, 1H, J=8 Hz), 7.13 (m, 2H), 6.95 (d, 1H, J=9 Hz), 3.58 (m, 4H), 3.55 (m, 4H), 2.55 (s, 3H), 2.39 (s, 3H). MS (M+1): 502.2

### Example 7

### 4-(5-(3,5-dimethylbenzofuran-2-carboxamido)pyridin-2-yl)-N-(2-fluorophenyl)piperazine-1-carboxamide (7)

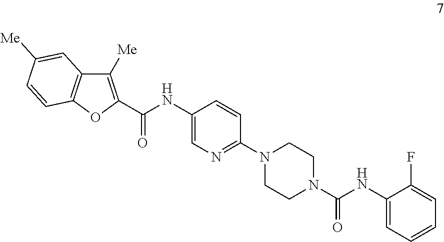

Compound 7 was prepared by the general procedure for compound 1 using intermediate B-5.

¹H NMR (500 MHz, DMSO-d6) δ 10.32 (s, 1H), 8.55 (s, 1H), 8.41 (s, 1H), 8.00 (dd, 1H, J=9, 1.5 Hz), 7.56 (s, 1H), 7.54 (d, 1H, J=8.5 Hz), 7.46 (m, 1H), 7.33 (d, 1H, J=8.5 Hz), 7.20 (m, 1H), 7.13 (m, 2H), 6.92 (d, 1H, J=9 Hz), 3.57 (m, 4H), 3.53 (m, 4H), 2.57 (s, 3H), 2.45 (s, 3H). MS (M+1): 488.2

### Example 8

### 4-(5-(3,7-dimethylbenzofuran-2-carboxamido)pyridin-2-yl)-N-(2-fluorophenyl)piperazine-1-carboxamide (8)

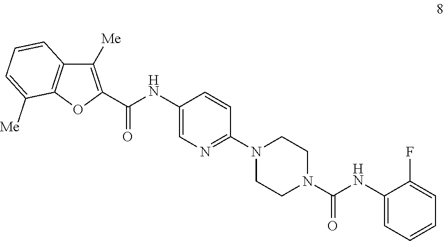

Compound 8 was prepared by the general procedure for compound 1 using intermediate B-5.

¹H NMR (500 MHz, DMSO-d6) δ 10.15 (s, 1H), 8.51 (d, 1H, J=3 Hz), 8.42 (s, 1H), 7.95 (dd, 1H, J=9, 2.5 Hz), 7.57 (d, 1H, J=7.5 Hz), 7.46 (m, 1H), 7.32 (d, 1H, J=7 Hz), 7.26 (t, 1H, J=7.5 Hz), 7.20 (m, 1H), 7.13 (m, 2H), 6.95 (d, 1H, J=9 Hz), 3.58 (m, 4H), 3.55 (m, 4H), 2.60 (s, 3H), 2.58 (s, 3H). MS (M+1): 488.2

### Example 9

### 4-(5-(7-fluoro-3-methylbenzofuran-2-carboxamido)pyridin-2-yl)-N-(2-fluorophenyl)piperazine-1-carboxamide (9)

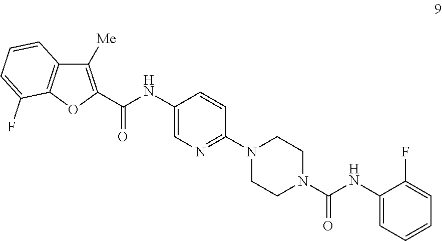

Compound 9 was prepared by the general procedure for compound 1 using intermediate B-5.

¹H NMR (500 MHz, DMSO-d6) δ 10.41 (s, 1H), 8.54 (s, 1H), 8.42 (s, 1H), 7.98 (d, 1H, J=9 Hz), 7.63 (d, 1H, J=7.5 Hz), 7.46 (m, 2H), 7.38 (m, 1H), 7.20 (m, 1H), 7.13 (m, 2H), 6.94 (d, 1H, J=9.5 Hz), 3.57 (m, 4H), 3.55 (m, 4H), 2.60 (s, 3H). MS (M+1): 492.2

### Example 10

### 4-(5-(benzo[b]thiophene-3-carboxamido)pyridin-2-yl)-N-(2-fluorophenyl)piperazine-1-carboxamide (10)

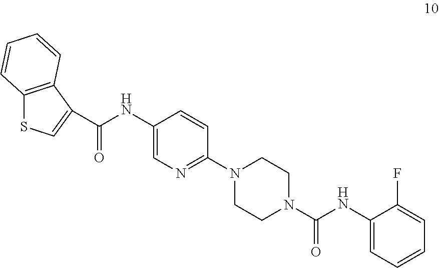

Compound 10 was prepared by the general procedure for compound 1 using intermediate B-5. ¹H NMR (500 MHz, DMSO-d6) δ 10.26 (s, 1H), 8.55 (s, 1H), 8.50 (d, 1H, J=2 Hz), 8.44 (d, 1H, J=7.5 Hz), 8.42 (s, 1H), 8.09 (d, 1H, J=8 Hz), 7.97 (dd, 1H, J=9, 2.5 Hz), 7.49 (t, 1H, J=7 Hz), 7.46 (m, 2H), 7.20 (m, 1H), 7.13 (m, 2H), 6.96 (d, 1H, J=9.5 Hz), 3.58 (m, 4H), 3.53 (m, 4H). MS (M+1): 476.3

### Example 11

### 4-(5-(benzofuran-3-carboxamido)pyridin-2-yl)-N-(2-fluorophenyl)piperazine-1-carboxamide (11)

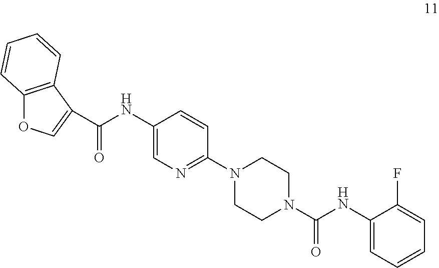

Compound 11 was prepared by the general procedure for compound 1 using intermediate B-5. ¹H NMR (500 MHz, DMSO-d6) δ 10.12 (s, 1H), 8.75 (s, 1H), 8.46 (d, 1H, J=2.5 Hz), 8.41 (s, 1H), 8.11 (d, 1H, J=7.5 Hz), 7.94 (dd, 1H, J=9.5, 2.5 Hz), 7.70 (d, 1H, J=7.5 Hz), 7.46 (m, 1H), 7.43 (m, 1H), 7.39 (td, 1H, J=7, 1.5 Hz), 7.20 (m, 1H), 7.13 (m, 2H), 6.96 (d, 1H, J=9 Hz), 3.58 (m, 4H), 3.53 (m, 4H). MS (M+1): 460.3

### Example 12

### N-(2-fluorophenyl)-4-(5-(3-methyl-5-phenylbenzofuran-2-carboxamido)pyridin-2-yl)piperazine-1-carboxamide (12)

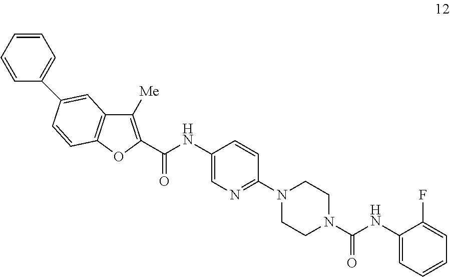

Compound 12 was prepared by the general procedure for compound 1 using intermediates A-3 and B-5. ¹H NMR (500 MHz, DMSO-d6) δ 10.41 (s, 1H), 8.57 (s, 1H), 8.42 (s, 1H), 8.04 (m, 2H), 7.81 (d, 1H, J=7.5 Hz), 7.75 (m, 3H), 7.51 (t, 2H, J=7.5 Hz), 7.46 (m, 1H), 7.40 (t, 1H, J=7 Hz), 7.20 (m, 1H), 7.13 (m, 2H), 6.94 (d, 1H, J=9 Hz), 3.58 (m, 4H), 3.53 (m, 4H), 2.66 (s, 3H). MS (M+1): 550.4

### Example 13

### N-(2-fluorophenyl)-4-(5-(3-methyl-6-phenylbenzofuran-2-carboxamido)pyridin-2-yl)piperazine-1-carboxamide (13)

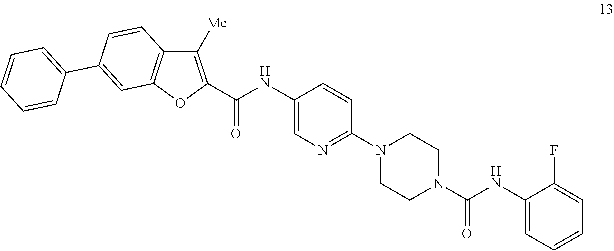

Compound 13 was prepared by the general procedure for compound 1 using intermediates A-10 and B-5. ¹H NMR (500 MHz, DMSO-d6) δ 10.41 (s, 1H), 8.56 (d, 1H, J=2.5 Hz), 8.42 (s, 1H), 8.02 (dd, 1H, J=9.5, 2.5 Hz), 7.87 (t, 2H, J=3.5 Hz), 7.77 (d, 2H, J=8.5 Hz), 7.70 (d, 1H, J=8.5 Hz), 7.53 (t, 2H, J=7.5 Hz), 7.46 (m, 1H), 7.42 (t, 1H, J=8 Hz), 7.20 (m, 1H), 7.13 (m, 2H), 6.94 (d, 1H, J=9 Hz), 3.57 (m, 4H), 3.54 (m, 4H), 2.63 (s, 3H). MS (M+1): 550.3

### Example 14

### ethyl 2-(4-(5-(3-methyl-5-phenylbenzofuran-2-carboxamido)pyridin-2-yl)piperazine-1-carboxamido)benzoate (14)

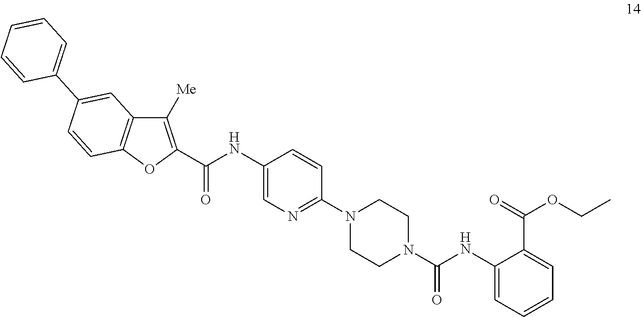

Compound 14 was prepared by the general procedure for compound 1 using intermediates A-3 and B-6. ¹H NMR (500 MHz, DMSO-d6) δ 10.46 (s, 1H), 10.42 (s, 1H), 8.57 (d, 1H, J=3 Hz), 8.35 (d, 1H, J=7.5 Hz), 8.05 (d, 1H, J=2 Hz), 8.03 (dd, 11H, J=9.5, 3 Hz), 7.96 (dd, 1H, J=8, 1.5 Hz), 7.81 (dd, 1H, J=8.5, 2 Hz), 7.75 (m, 3H), 7.58 (t, 1H, J=9 Hz), 7.51 (t, 2H, J=7.5 Hz), 7.40 (t, 1H, J=7.5 Hz), 7.07 (t, 1H, J=8.5 Hz), 6.93 (d, 1H, J=9 Hz), 4.36 (q, 211, J=7 Hz), 3.61 (m, 8H), 3.34 (s, 3H), 2.65 (s, 3H), 1.35 (t, 3H, J=7 Hz). MS (M+1): 604.3

### Example 15

### methyl 3-((1-(5-(3-methyl-5-phenylbenzofuran-2-carboxamido)pyridin-2-yl)piperidin-4-yl)methoxy)benzoate (15)

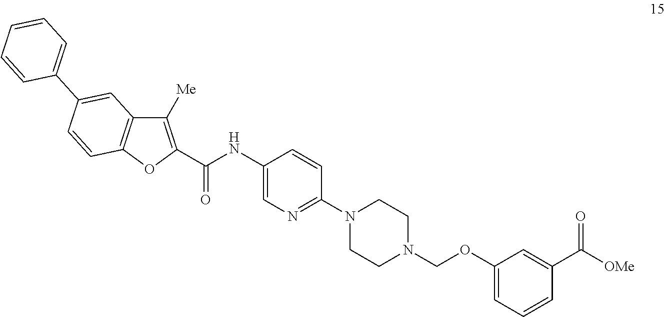

Compound 15 was prepared by the general procedure for compound 1 using intermediates A-3 and B-13. ¹H NMR (500 MHz, DMSO-d6) δ 10.36 (s, 1H), 8.51 (d, 1H, J=3 Hz), 8.04 (s, 1H), 7.96 (dd, 1H, J=8.5, 2.5 Hz), 7.81 (d, 1H, J=8.5 Hz), 7.76 (d, 2H, J=8 Hz), 7.73 (d, 1H, J=9 Hz), 7.55 (d, 1H, J=7.5 Hz), 7.51 (t, 2H, J=7.5 Hz), 7.46 (s, 1H), 7.44 (t, 1H, J=8 Hz), 7.39 (t, 1H, J=7 Hz), 7.25 (dd, 1H, J=8, 3 Hz), 6.88 (d, 1H, J=9.5 Hz), 4.32 (d, 2H, J=13 Hz), 3.93 (d, 2H, J=6.5 Hz), 3.86 (s, 3H), 2.83 (t, 2H, J=11 Hz), 2.65 (s, 3H), 2.03 (m, 1H), 1.86 (d, 2H, J=10.5 Hz), 1.33 (qd, 2H, J=12, 3.5 Hz). MS (M+1): 576.4

### Example 16

### methyl 3-((1-(5-(3-methyl-5-phenylbenzofuran-2-carboxamido)pyridin-2-yl)piperidin-4-yloxy)methyl)benzoate (16)

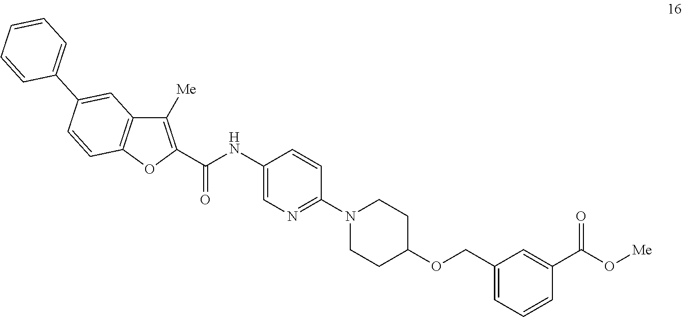

Compound 16 was prepared by the general procedure for compound 1 using intermediates A-3 and B-9. ¹H NMR (500 MHz, DMSO-d6) δ 10.38 (s, 1H), 8.52 (d, 1H, J=2.5 Hz), 8.05 (s, 1H), 7.97 (d, 1H, J=10 Hz), 7.95 (s, 1H), 7.88 (d, 1H, J=8 Hz), 7.81 (d, 1H, J=8.5 Hz), 7.76 (m, 3H), 7.64 (d, 1H, J=7.5 Hz), 7.53 (d, 1H, J=7 Hz), 7.51 (t, 2H, J=7.5 Hz), 7.40 (t, 1H, J=7.5 Hz), 6.91 (d, 1H, J=9.5 Hz), 4.64 (s, 2H), 3.96 (d, 2H, J=13.5 Hz), 3.87 (s, 3H), 3.67 (m, 1H), 3.16 (t, 2H, J=10 Hz), 2.65 (s, 3H), 1.97 (m, 2H), 1.54 (m, 2H). MS (M+1): 576.3

### Example 17

### 3-((1-(5-(3-methyl-5-phenylbenzofuran-2-carboxamido)pyridin-2-yl)piperidin-4-yloxy)methyl)benzoic acid (17)

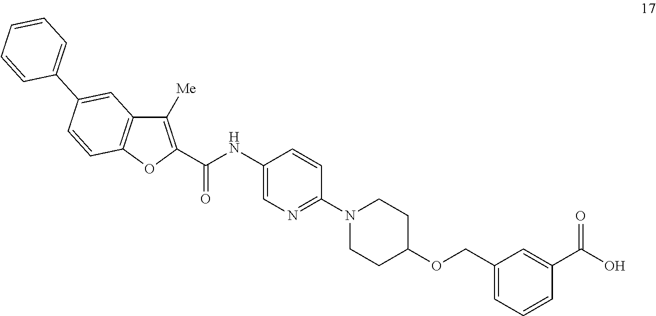

Compound 17 was prepared by the hydrolysis of compound 16.

¹H NMR (500 MHz, DMSO-d6) δ 10.36 (s, 1H), 8.51 (s, 1H), 8.05 (s, 1H), 7.96 (d, 1H, J=9.5 Hz), 7.94 (s, 1H), 7.86 (d, 1H, J=7.5 Hz), 7.80 (d, 1H, J=9 Hz), 7.76 (d, 211, J=7.5 Hz), 7.73 (d, 1H, J=8.5 Hz), 7.56 (d, 1H, J=6 Hz), 7.50 (t, 2H, J=8 Hz), 7.45 (t, 1H, J=8 Hz), 7.39 (t, 1H, J=7 Hz), 6.89 (d, 1H, J=9 Hz), 4.62 (s, 2H), 3.97 (d, 2H, J=13.5 Hz), 3.66 (m, 1H), 3.15 (t, 2H, J=10 Hz), 2.65 (s, 3H), 1.96 (m, 2H), 1.53 (m, 2H). MS (M+1): 562.4

### Example 18

### 3-((1-(5-(3-methyl-5-phenylbenzofuran-2-carboxamido)pyridin-2-yl)piperidin-4-yl)methoxy)benzoic acid (18)

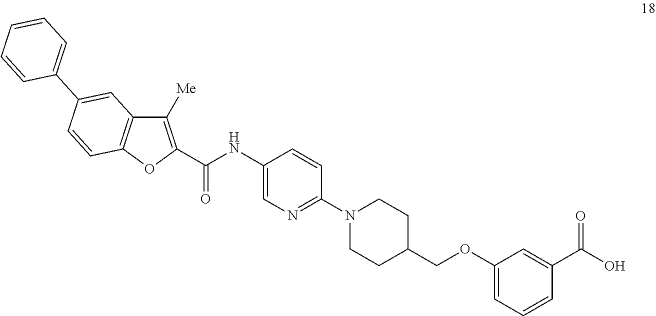

Compound 18 was prepared by the hydrolysis of compound 15.

¹H NMR (500 MHz, DMSO-d6) δ 10.36 (s, 1H), 8.51 (d, 1H, J=2.5 Hz), 8.04 (s, 1H), 7.96 (dd, 1H, 9, 2.5 Hz), 7.80 (d, 1H, J=9 Hz), 7.76 (d, 2H, J=8 Hz), 7.73 (d, 1H, J=8.5 Hz), 7.51 (t, 3H, J=7.5 Hz), 7.46 (s, 1H), 7.39 (t, 1H, J=7 Hz), 7.36 (t, 1H, J=7.5 Hz), 7.13 (d, 1H, J=8 Hz), 6.88 (d, 1H, J=9 Hz), 4.62 (d, 2H, J=13 Hz), 3.91 (d, 2H, J=6.5 Hz), 2.83 (t, 2H, J=12 Hz), 2.65 (s, 3H), 2.03 (m, 1H), 1.86 (d, 2H, J=11.5 Hz), 1.32 (q, 2H, J=12 Hz). MS (M+1): 562.4

### Example 19

### 2-(4-(5-(3-methyl-5-phenylbenzofuran-2-carboxamido)pyridin-2-yl)piperazine-1-carboxamido)benzoic acid (19)

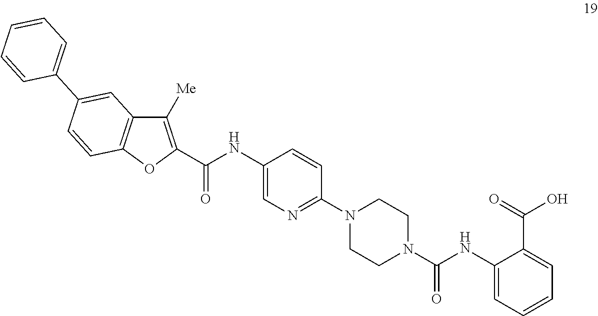

Compound 19 was prepared by the hydrolysis of compound 14.

¹H NMR (500 MHz, DMSO-d6) δ 10.97 (s, 1H), 10.69 (s, 1H), 8.65 (s, 1H), 8.44 (d, 1H, J=8.5 Hz), 8.26 (d, 1H, 9 Hz), 8.07 (s, 1H), 7.98 (d, 1H, J=8 Hz), 7.83 (d, 1H, J=7.5 Hz), 7.75 (m, 3H), 7.56 (t, 1H, J=7.5 Hz), 7.51 (t, 2H, J=7.5 Hz), 7.40 (t, 1H, J=7.5 Hz), 7.29 (broad s, 1H), 7.05 (t, 1H, J=7.5 Hz), 3.77 (broad s, 4H), 3.70 (broad s, 4H), 2.67 (s, 3H). MS (M+1): 576.4

### Example 20

### N-(2-fluorophenyl)-4-(5-(5-(2-fluorophenyl)-3-methylbenzofuran-2-carboxamido)pyridin-2-yl)piperazine-1-carboxamide (20)

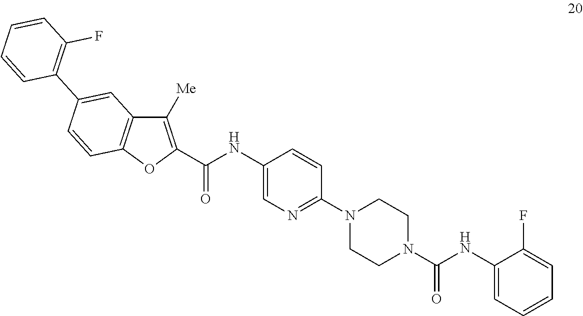

Compound 20 was prepared by the general procedure for compound 1 using intermediates A-4 and B-5. ¹H NMR (500 MHz, DMSO-d6) δ 10.44 (s, 1H), 8.57 (d, 1H, J=2.5 Hz), 8.42 (s, 1H), 8.03 (dd, 1H, J=8.5, 2.5 Hz), 7.94 (s, 1H), 7.76 (d, 1H, J=9 Hz), 7.69 (d, 1H, J=8 Hz), 7.64 (t, 1H, J=7 Hz), 7.46 (m, 2H), 7.36 (m, 2H), 7.20 (m, 1H), 7.13 (m, 2H), 6.94 (d, 111, J=9 Hz), 3.58 (m, 4H), 3.54 (m, 4H), 2.63 (s, 3H). MS (M+1): 568.2

### Example 21

### ethyl 2-(4-(5-(5-(2-fluorophenyl)-3-methylbenzofuran-2-carboxamido)pyridin-2-yl)piperazine-1-carboxamido)benzoate (21)

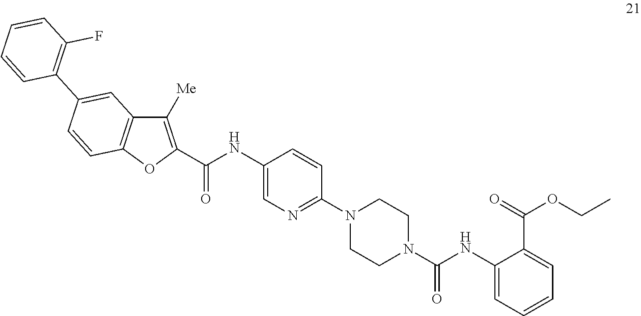

Compound 21 was prepared by the general procedure for compound 1 using intermediates A-4 and B-6. ¹H NMR (500 MHz, DMSO-d6) δ 10.45 (s, 2H), 8.58 (d, 1H, J=2.5 Hz), 8.35 (d, 1H, J=8 Hz), 8.05 (d, 1H, J=9 Hz), 7.96 (d, 1H, J=8 Hz), 7.94 (s, 1H), 7.76 (d, 1H, J=9 Hz), 7.68 (d, 1H, J=8.5 Hz), 7.63 (t, 1H, J=8 Hz), 7.58 (t, 1H, J=8.5 Hz), 7.46 (q, 1H, J=6.5 Hz), 7.37 (d, 1H, J=8.5 Hz), 7.34 (d, 1H, J=7.5 Hz), 7.07 (t, 1H, J=7.5 Hz), 6.97 (d, 1H, J=9 Hz), 4.36 (q, 2H, J=7.5 Hz), 3.62 (broad s, 8H), 2.63 (s, 3H), 1.35 (t, 3H, J=7.5 Hz). MS (M+1): 622.3

### Example 22

### methyl 3-((1-(5-(5-(2-fluorophenyl)-3-methylbenzofuran-2-carboxamido)pyridin-2-yl)piperidin-4-yloxy)methyl)benzoate (22)

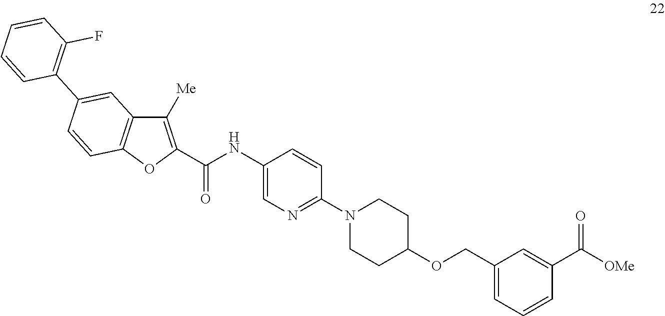

Compound 22 was prepared by the general procedure for compound 1 using intermediates A-4 and B-9. ¹H NMR (500 MHz, DMSO-d6) δ 10.39 (s, 1H), 8.52 (d, 1H, J=2.5 Hz), 7.96 (m, 1H), 7.96 (s, 1H), 7.94 (s, 1H), 7.88 (d, 1H, J=7.5 Hz), 7.76 (d, 1H, J=9 Hz), 7.68 (d, 1H, J=8.5 Hz), 7.63 (m, 2H), 7.52 (t, 1H, J=7.5 Hz), 7.46 (m, 1H), 7.37 (d, 1H, J=9 Hz), 7.34 (d, 1H, J=7.5 Hz), 6.90 (d, 1H, J=9.5 Hz), 4.65 (s, 2H), 3.97 (m, 2H), 3.87 (s, 3H), 3.67 (m, 1H), 3.16 (t, 2H, J=10 Hz), 2.63 (s, 3H), 1.97 (m, 2H), 1.53 (q, 2H, J=9.5 Hz). MS (M+1): 594.3

### Example 23

### methyl 3-((1-(5-(5-(2-fluorophenyl)-3-methylbenzofuran-2-carboxamido)pyridin-2-yl)piperidin-4-yl)methoxy)benzoate (23)

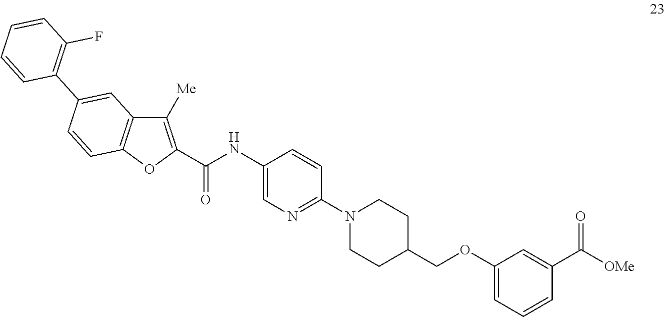

Compound 23 was prepared by the general procedure for compound 1 using intermediates A-4 and 13-13. ¹H NMR (500 MHz, DMSO-d6) δ 10.38 (s, 1H), 8.51 (d, 1H, J=2.5 Hz), 7.96 (dd, 1H, J=9.5, 2.5 Hz), 7.94 (s, 1H), 7.76 (d, 1H, J=8.5 Hz), 7.68 (d, 1H, J=8.5 Hz), 7.63 (t, 1H, J=7 Hz), 7.55 (d, 1H, J=7.5 Hz), 7.46 (m, 3H), 7.37 (d, 1H, J=9 Hz), 7.34 (d, 1H, J=7.5 Hz), 7.25 (dd, 1H, J=8.5, 2.5 Hz), 6.89 (d, 1H, J=9 Hz), 4.32 (d, 2H, J=13 Hz), 3.94 (d, 2H, J=7 Hz), 3.86 (s, 3H), 2.83 (t, 2H, J=12.5 Hz), 2.63 (s, 3H), 2.04 (m, 1H), 1.87 (d, 2H, J=12.5 Hz), 1.33 (qd, 2H, J=11.5, 3 Hz). MS (M+1): 594.4

### Example 24

### 2-(4-(5-(5-(2-fluorophenyl)-3-methylbenzofuran-2-carboxamido)pyridin-2-yl)piperazine-1-carboxamido)benzoic acid (24)

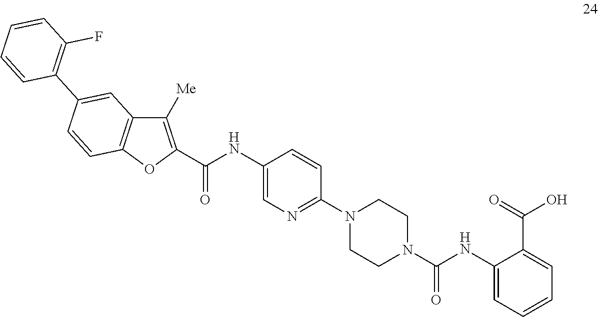

Compound 24 was prepared by the hydrolysis of compound 21.

¹H NMR (500 MHz, DMSO-d6) δ 10.97 (s, 1H), 10.66 (broad s, 1H), 8.63 (s, 1H), 8.44 (d, 1H, J=8.5 Hz), 8.21 (broad s, 1H), 7.98 (d, 1H, J=7.5 Hz), 7.96 (s, 1H), 7.77 (d, 1H, J=8.5 Hz), 7.70 (d, 1H, J=9 Hz), 7.63 (t, 1H, J=6.5 Hz), 7.56 (t, 1H, J=8 Hz), 7.46 (q, TH, J=7 Hz), 7.37 (d, 1H, J=8.5 Hz), 7.34 (d, 1H, J=7.5 Hz), 7.21 (broad s, 1H), 7.04 (t, 1H, J=7.5 Hz), 3.72 (broad s, 4H), 3.69 (broad s, 4H), 2.64 (s, 3H). MS (M+1): 594.4

### Example 25

### 3-((1-(5-(5-(2-fluorophenyl)-3-methyl benzofuran-2-carboxamido)pyridin-2-yl)piperidin-4-yloxy)methyl)benzoic acid (25)

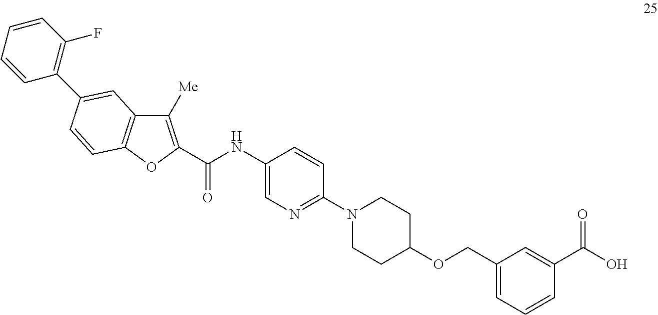

Compound 25 was prepared by the hydrolysis of compound 22.

¹H NMR (500 MHz, DMSO-d6) δ 10.79 (broad s, 1H), 8.64 (s, 1H), 8.29 (d, 1H, J=9.5 Hz), 7.96 (d, 2H, J=5 Hz), 7.87 (d, 1H, J=7 Hz), 7.78 (d, 1H, J=8.5 Hz), 7.71 (d, 1H, J=7.5 Hz), 7.63 (m, 2H), 7.50 (t, 1H, J=7.5 Hz), 7.46 (m, 2H), 7.37 (d, 1H, J=8.5 Hz), 7.34 (d, 1H, J=7.5 Hz), 4.65 (s, 2H), 3.96 (m, 2H), 3.76 (m, 1H), 3.52 (m, 2H), 2.64 (s, 3H), 2.02 (m, 2H), 1.69 (m, 2H). MS (M+1): 580.4

### Example 26

### 3-((1-(5-(5-(2-fluorophenyl)-3-methylbenzofuran-2-carboxamido)pyridin-2-yl)piperidin-4-yl)methoxy)benzoic acid (26)

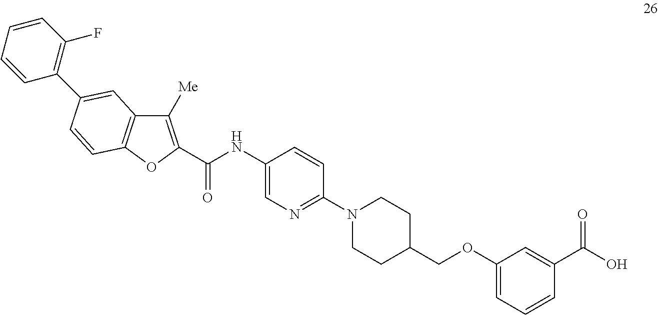

Compound 26 was prepared by the hydrolysis of compound 23.

¹H NMR (500 MHz, DMSO-d6) δ 10.71 (broad s, 1H), 8.60 (s, 1H), 8.23 (broad s, 1H), 7.96 (s, 1H), 7.78 (d, 1H, J=8.5 Hz), 7.70 (d, 1H, J=8.5 Hz), 7.63 (t, 1H, J=8 Hz), 7.54 (d, 1H, J=7.5 Hz), 7.46 (m, 2H), 7.42 (t, 1H, J=8 Hz), 7.36 (m, 3H), 7.21 (dd, 1H, J=8.5, 2.5 Hz), 4.30 (d, 2H, J=13.5 Hz), 3.95 (d, 2H, J=6.5 Hz), 3.14 (m, 2H), 2.64 (s, 3H), 2.14 (m, 1H), 1.93 (d, 2H, 12 Hz), 1.41 (q, 2H, J=11.5 Hz). MS (M+1): 580.4

### Example 27

### N-(2-fluorophenyl)-4-(5-(5-(3-fluorophenyl)-3-methylbenzofuran-2-carboxamido)pyridin-2-yl)piperazine-1-carboxamide (27)

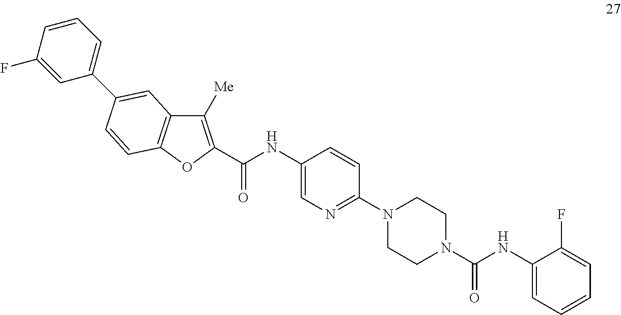

Compound 27 was prepared by the general procedure for compound 1 using intermediates A-5 and B-5. ¹H NMR (500 MHz, DMSO-d6) δ 10.61 (broad s, 1H), 8.59 (d, 1H, J=2.5 Hz), 8.45 (s, 1H), 8.19 (d, 1H, J=7 Hz), 8.15 (s, 1H), 7.87 (d, 1H, J=8.5 Hz), 7.75 (d, 1H, J=9 Hz), 7.64 (m, 2H), 7.55 (q, 1H, J=7 Hz), 7.46 (m, 1H), 7.22 (m, 3H), 7.14 (m, 2H), 3.63 (s, 8H), 2.67 (s, 3H). MS (M+1): 568.4

### Example 28

### methyl 3-((1-(5-(5-(3-fluorophenyl)-3-methylbenzofuran-2-carboxamido)pyridin-2-yl)piperidin-4-yloxy)methyl)benzoate (28)

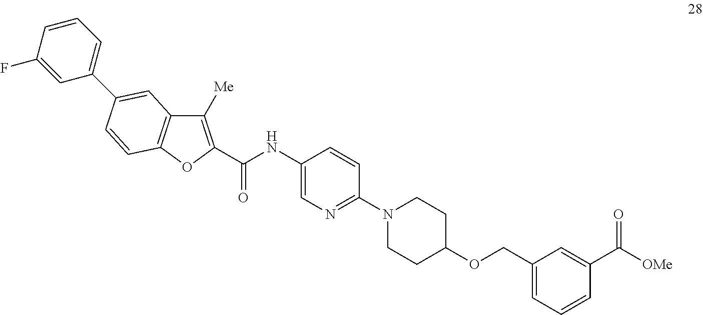

Compound 28 was prepared by the general procedure for compound 1 using intermediates A-5 and B-9. ¹H NMR (500 MHz, DMSO-d6) δ 10.59 (broad s, 1H), 8.54 (d, 1H, J=2.5 Hz), 8.15 (s, 1H), 8.13 (broad s, 1H), 7.96 (s, 1H), 7.89 (d, 1H, J=7.5 Hz), 7.86 (dd, 1H, J=8.5, 1.5 Hz), 7.75 (d, 1H, 8.5 Hz), 7.64 (m, 3H), 7.55 (m, 2H), 7.23 (td, 2H, J=8.5, 2.5 Hz), 4.66 (s, 2H), 3.93 (m, 2H), 3.87 (s, 3H), 3.72 (m, 1H), 3.33 (m, 2H), 2.66 (s, 3H), 2.00 (m, 2H), 1.62 (m, 2H). MS (M+1): 594.4

### Example 29

### 3-((1-(5-(5-(3-fluorophenyl)-3-methylbenzofuran-2-carboxamido)pyridin-2-yl)piperidin-4-yloxy)methyl)benzoic acid (29)

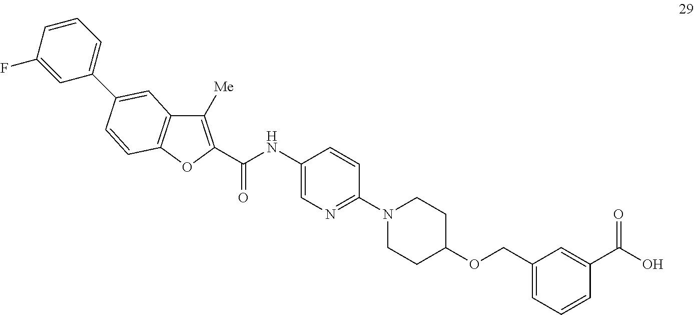

Compound 29 was prepared by the hydrolysis of compound 28.

¹H NMR (500 MHz, DMSO-d6) δ 10.75 (broad s, 1H), 8.62 (s, 1H), 8.26 (d, 1H, J=8 Hz), 8.16 (s, 1H), 7.95 (s, 1H), 7.87 (d, 2H, J=8 Hz), 7.75 (d, 1H, J=8.5 Hz), 7.63 (m, 3H), 7.55 (t, 1H, J=8 Hz), 7.50 (t, 1H, J=8 Hz), 7.41 (broad s, 1H), 7.23 (t, 1H, J=9 Hz), 4.65 (s, 2H), 3.96 (m, 2H), 3.76 (m, 1H), 3.49 (m, 2H), 2.67 (s, 3H), 2.02 (m, 2H), 1.68 (m, 2H). MS (M+1): 580.3

### Example 30

### ethyl 2-(4-(5-(5-(3-fluorophenyl)-3-methylbenzofuran-2-carboxamido)pyridin-2-yl)piperazine-1-carboxamido)benzoate (30)

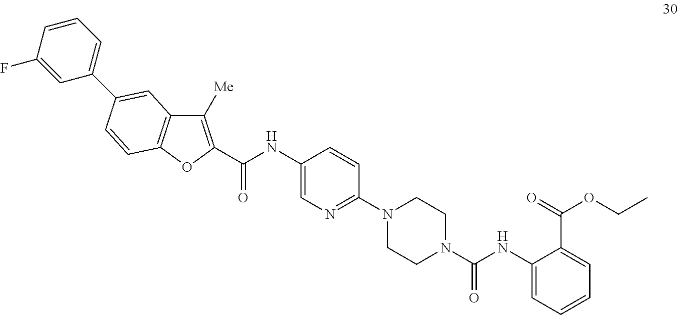

Compound 30 was prepared by the general procedure for compound 1 using intermediates A-5 and B-6. ¹H NMR (500 MHz, DMSO-d6) δ 10.46 (s, 1H), 10.42 (s, 1H), 8.57 (d, 1H, J=2.5 Hz), 8.35 (d, 1H, J=8.5 Hz), 8.13 (s, 1H), 8.02 (dd, 1H, J=9, 2.5 Hz), 7.96 (d, 1H, J=8 Hz), 7.85 (d, 1H, J=9 Hz), 7.74 (d, 1H, J=9 Hz), 7.64 (m, 2H), 7.58 (t, 1H, J=6.5 Hz), 7.54 (q, 1H, J=6.5 Hz), 7.22 (td, 1H, J=8.5, 2 Hz), 7.07 (t, 1H, J=7.5 Hz), 6.93 (d, 1H, J=9 Hz), 4.36 (q, 2H, J=7 Hz), 3.61 (m, 8H), 2.65 (s, 3H), 1.35 (t, 3H, J=7.5 Hz). MS (M+1): 622.4

### Example 31

### 2-(4-(5-(5-(3-fluorophenyl)-3-methylbenzofuran-2-carboxamido)pyridin-2-yl)piperazine-1-carboxamido)benzoic acid (31)

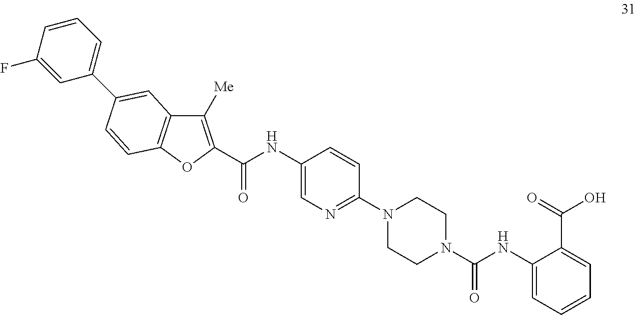

Compound 31 was prepared by the hydrolysis of compound 30.

¹H NMR (500 MHz, DMSO-d6) δ 10.97 (s, 1H), 10.69 (broad s, 1H), 8.64 (d, 1H, J=2 Hz), 8.44 (d, 1H, J=8 Hz), 8.24 (d, 1H, J=7.5 Hz), 8.15 (s, 1H), 7.98 (dd, 1H, J=8, 2 Hz), 7.87 (dd, 1H, J=8.5, 2 Hz), 7.75 (d, 1H, J=8.5 Hz), 7.64 (m, 2H), 7.55 (m, 2H), 7.26 (broad s, 1H), 7.23 (td, 1H, J=8.5, 2 Hz), 7.05 (t, 1H, J=8 Hz), 3.75 (broad s, 4H), 3.70 (broad s, 4H), 2.67 (s, 3H). MS (M+1): 594.3

### Example 32

### methyl 3-((1-(5-(5-(3-fluorophenyl)-3-methylbenzofuran-2-carboxamido)pyridin-2-yl)piperidin-4-yl)methoxy)benzoate (32)

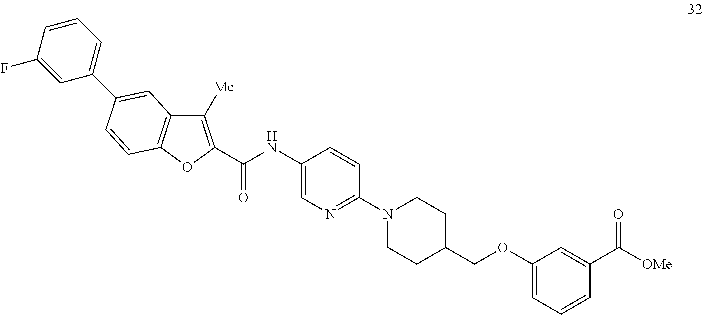

Compound 32 was prepared by the general procedure for compound 1 using intermediates A-5 and B-13. ¹H NMR (500 MHz, DMSO-d6) δ 10.36 (s, 1H), 8.51 (d, 1H, J=2.5 Hz), 8.12 (s, 1H), 7.95 (dd, 1H, J=9, 2 Hz), 7.85 (d, 1H, J=8.5 Hz), 7.74 (d, 1H, J=8.5 Hz), 7.64 (m, 2H), 7.55 (m, 2H), 7.45 (s, 1H), 7.44 (t, 1H, J=8 Hz), 7.25 (dd, 1H, J=8.5, 2.5 Hz), 7.22 (td, 1H, J=8, 2 Hz), 6.88 (d, 1H, J=9.5 Hz), 4.32 (d, 2H, J=13 Hz), 3.93 (d, 2H, J=6.5 Hz), 3.85 (s, 3H), 2.83 (t, 2H, J=12 Hz), 2.65 (s, 3H), 2.03 (m, 1H), 1.86 (d, 211, J=11 Hz), 1.33 (q, 2H, J=12 Hz). MS (M+1): 594.4

### Example 33

### 3-((1-(5-(5-(3-fluorophenyl)-3-methylbenzofuran-2-carboxamido)pyridin-2-yl)piperidin-4-yl)methoxy)benzoic acid (33)

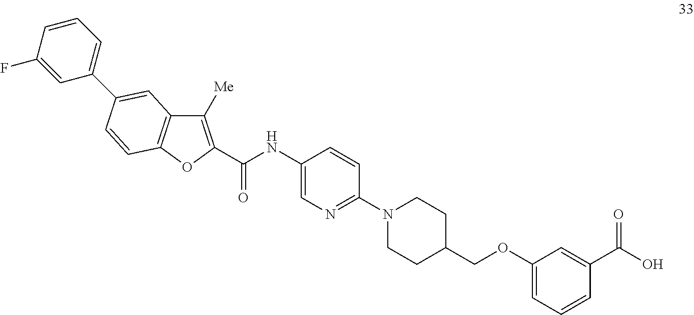

Compound 33 was prepared by the hydrolysis of compound 32.

¹H NMR (500 MHz, DMSO-d6) δ 10.64 (broad s, 1H), 8.59 (s, 1H), 8.18 (broad s, 1H), 8.15 (s, 1H), 7.87 (d, 1H, J=9 Hz), 7.75 (d, 1H, J=8.5 Hz), 7.64 (m, 2H), 7.54 (m, 2H), 7.46 (m, 1H), 7.42 (t, 1H, J=8 Hz), 7.23 (m, 3H), 4.30 (d, 2H, J=13 Hz), 3.94 (d, 2H, J″ 6 Hz), 3.08 (broad s, 2H), 2.66 (s, 3H), 2.12 (m, 1H), 1.92 (d, 2H, J=11 Hz), 1.40 (q, 2H, J=9.5 Hz). MS (M+1): 580.6

### Example 34

### methyl 3-((1-(5-(5-(4-fluorophenyl)-3-methylbenzofuran-2-carboxamido)pyridin-2-yl)piperidin-4-yl)methoxy)benzoate (34)

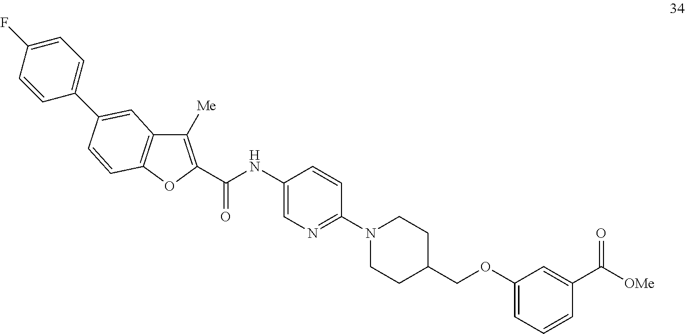

Compound 34 was prepared by the general procedure for compound 1 using intermediates A-6 and B-13. ¹H NMR (500 MHz, DMSO-d6) δ 10.43 (s, 1H), 8.52 (d, 1H, J=2.5 Hz), 8.04 (s, 1H), 8.03 (d, 1H, J=9.5 Hz), 7.80 (m, 3H), 7.73 (d, 1H, J=9 Hz), 7.55 (d, 1H, J=7.5 Hz), 7.46 (s, 1H), 7.43 (d, 1H, J=8.5 Hz), 7.33 (t, 2H, J=8.5 Hz), 7.25 (dd, 1H, J=8.5, 2.5 Hz), 7.00 (broad s, 1H), 4.30 (d, 2H, J=13 Hz), 3.94 (d, 2H, J=6.5 Hz), 3.85 (s, 3H), 2.90 (t, 2H, J=12.5 Hz), 2.64 (s, 3H), 2.06 (m, 1H), 1.88 (d, 2H, J=12 Hz), 1.35 (q, 2H, J=12 Hz). MS (M+1): 594.6

### Example 35

### methyl 3-((1-(5-(5-(4-fluorophenyl)-3-methylbenzofuran-2-carboxamido)pyridin-2-yl)piperidin-4-yloxy)methyl)benzoate (35)

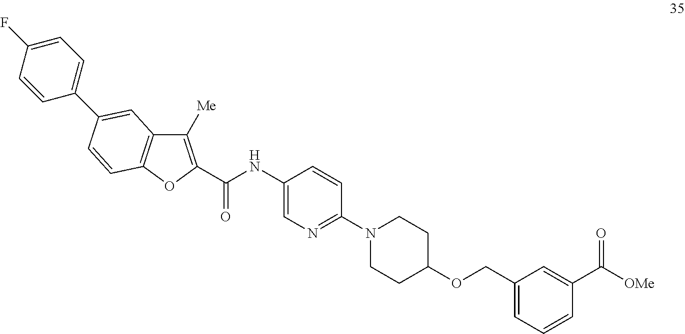

Compound 35 was prepared by the general procedure for compound 1 using intermediates A-6 and B-9. ¹H NMR (500 MHz, DMSO-d6) δ 10.49 (broad s, 1H), 8.54 (s, 1H), 8.07 (m, 1H), 8.05 (s, 1H), 7.96 (s, 1H), 7.89 (d, 1H, J=7.5 Hz), 7.80 (m, 3H), 7.73 (d, 1H, J=8.5 Hz), 7.65 (d, 1H, J=8 Hz), 7.53 (t, 1H, J=7.5 Hz), 7.34 (t, 2H, J=9 Hz), 7.10 (broad s, 1H), 4.65 (s, 2H), 3.94 (m, 2H), 3.87 (s, 3H), 3.70 (m, 1H), 3.27 (m, 2H), 2.65 (s, 3H), 1.99 (m, 2H), 1.59 (m, 2H). MS (M+1): 594.6

### Example 36

### 2-(4-(5-(5-(4-fluorophenyl)-3-methylbenzofuran-2-carboxamido)pyridin-2-yl)piperazine-1-carboxamido)benzoic acid (36)

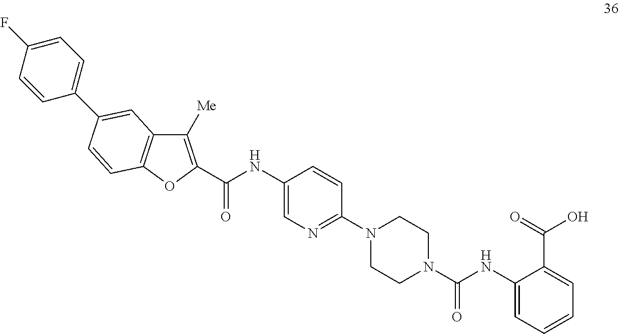

Compound 36 was prepared by the hydrolysis of compound 38.

¹H NMR (500 MHz, DMSO-d6) δ 13.60 (broad s, 1H), 10.98 (s, 1H), 10.46 (s, 1H), 8.57 (d, 1H, J=2 Hz), 8.44 (d, 1H, J=8.5 Hz), 8.06 (d, 1H, J=9 Hz), 8.05 (s, 1H), 7.98 (d, 1H, J=7.5 Hz), 7.80 (m, 3H), 7.73 (d, 1H, J=8.5 Hz), 7.56 (t, 1H, J=8.5 Hz), 7.33 (t, 2H, J=8.5 Hz), 7.04 (t, 1H, J=7.5 Hz), 6.99 (d, 1H, J=8.5 Hz), 3.63 (s, 8H), 2.65 (s, 3H). MS (M+1): 594.6

### Example 37

### 3-((1-(5-(5-(4-fluorophenyl)-3-methylbenzofuran-2-carboxamido)pyridin-2-yl)piperidin-4-yloxy)methyl)benzoic acid (37)

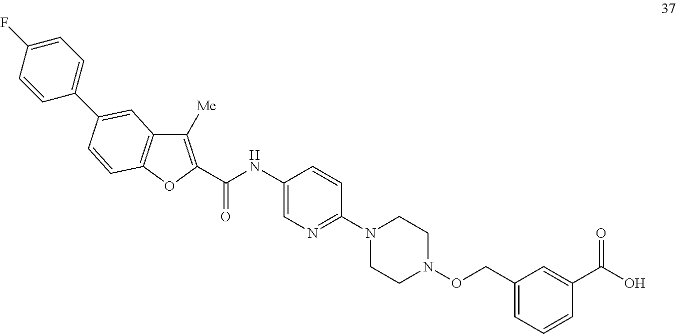

Compound 37 was prepared by the hydrolysis of compound 35.

¹H NMR (500 MHz, DMSO-d6) δ 10.6 5 (broad s, 1H), 8.58 (s, 1H), 8.19 (broad s, 1H), 8.06 (s, 1H), 7.95 (s, 1H), 7.87 (d, 2H, J=8 Hz), 7.80 (m, 3H), 7.74 (d, 1H, J=8.5 Hz), 7.62 (d, 1H, J=7.5 Hz), 7.50 (t, 1H, J=7.5 Hz), 7.34 (t, 2H, J=8.5 Hz), 7.29 (broad s, 1H), 4.65 (s, 2H), 3.94 (m, 2H), 3.73 (m, 1H), 3.40 (m, 2H), 2.65 (s, 3H), 2.00 (m, 2H), 1.64 (m, 2H). MS (M+1): 580.6

### Example 38

### ethyl 2-(4-(5-(5-(4-fluorophenyl)-3-methylbenzofuran-2-carboxamido)pyridin-2-yl)piperazine-1-carboxamido)benzoate (38)

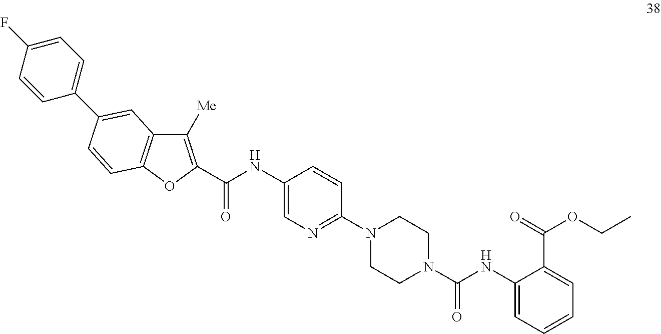

Compound 38 was prepared by the general procedure for compound 1 using intermediates A-6 and B-6. ¹H NMR (500 MHz, DMSO-d6) δ 10.46 (s, 1H), 10.42 (s, 1H), 8.57 (d, 1H, J=3 Hz), 8.35 (d, 1H, J=8.5 Hz), 8.04 (s, 1H), 8.02 (d, 1H, J=8.5 Hz), 7.96 (d, 1H, J=8 Hz), 7.80 (m, 3H), 7.73 (d, 1H, J=8.5 Hz), 7.58 (t, 1H, J=8.5 Hz), 7.33 (t, 2H, J=9 Hz), 7.07 (t, 1H, J=8 Hz), 6.94 (d, 1H, J=9 Hz), 4.36 (q, 2H, J=7.5 Hz), 3.61 (m, 8H), 2.65 (s, 3H), 1.35 (t, 31-1, J=7 Hz). MS (M+1): 622.6

### Example 39

### 3-((1-(5-(5-(4-fluorophenyl)-3-methylbenzofuran-2-carboxamido)pyridin-2-yl)piperidin-4-yl)methoxy)benzoic acid (39)

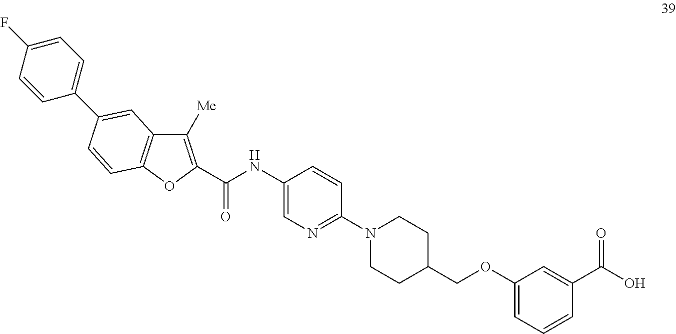

Compound 39 was prepared by the hydrolysis of compound 34.

¹H NMR (500 MHz, DMSO-d6) δ 10.35 (s, 1H), 8.51 (d, 1H, J=2 Hz), 8.03 (s, 1H), 7.95 (dd, 1H, J=9, 2 Hz), 7.80 (m, 3H), 7.72 (d, 1H, J=9 Hz), 7.52 (d, 1H, J=7.5 Hz), 7.45 (s, 1H), 7.39 (t, 1H, J=8 Hz), 7.33 (t, 2H, J=9 Hz), 7.18 (d, 1H, J=8 Hz), 6.88 (d, 1H, J=9.5 Hz), 4.32 (d, 2H, J=13.5 Hz), 3.92 (d, 2H, J=6.5 Hz), 2.83 (t, 2H, J=11.5 Hz), 2.64 (s, 3H), 2.03 (m, 1H), 1.86 (d, 2H, J=11.5 Hz), 1.32 (qd, 2H, J=12, 3.5 Hz). MS (M+1): 580.4

### Example 40

### N-(2-fluorophenyl)-4-(5-(5-(4-fluorophenyl)-3-methylbenzofuran-2-carboxamido)pyridin-2-yl)piperazine-1-carboxamide (40)

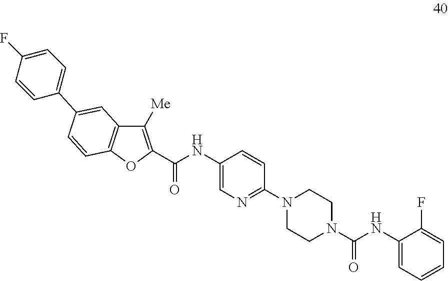

Compound 40 was prepared by the general procedure for compound 1 using intermediates A-6 and B-5. ¹H NMR (500 MHz, DMSO-d6) δ 10.41 (s, 1H), 8.56 (d, 1H, J=2.5 Hz), 8.42 (s, 1H), 8.04 (s, 1H), 8.02 (dd, 1H, J=9.5, 2.5 Hz), 7.80 (m, 3H), 7.73 (d, 1H, J=8.5 Hz), 7.46 (m, 1H), 7.34 (t, 2H, J=8.5 Hz), 7.20 (m, 1H), 7.13 (m, 2H), 6.94 (d, 1H, f=9 Hz), 3.57 (m, 4H), 3.54 (m, 4H), 2.65 (s, 3H). MS (M+1): 568.4

### Example 41

### N-(2-fluorophenyl)-4-(5-(benzofuran-2-carboxamido)pyridin-2-yl)piperazine-1-carboxamide

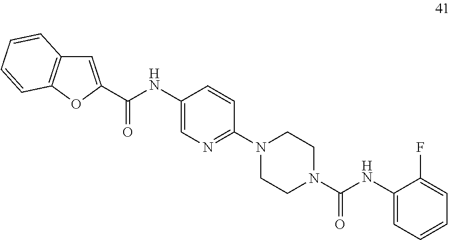

Compound 41 was prepared by the general procedure for compound 1 using intermediate B-5. ¹H NMR (500 MHz, DMSO-d6) δ 10.52 (s, 1H), 8.55 (s, 1H), 8.42 (s, 1H), 7.99 (dd, 1H, J=9, 3 Hz), 7.83 (d, 1H, J=7.5 Hz), 7.73 (t, 2H, J=5 Hz), 7.52 (t, 1H, J=7.5 Hz), 7.45 (m, 1H), 7.38 (t, 1H, J=7.5 Hz), 7.20 (m, 1H), 7.13 (m, 2H), 6.95 (d, 1H, J=9 Hz), 3.57 (m, 4H), 3.54 (m, 4H). MS (M+1): 460.3

### Example 42

### N-(2-fluorophenyl)-4-(5-(3-methylbenzo[b]thiophene-2-carboxamido)pyridin-2-yl)piperazine-1-carboxamide (42)

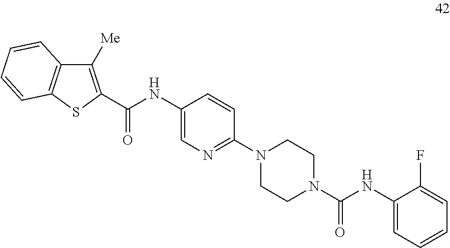

Compound 42 was prepared by the general procedure for compound 1 using intermediate B-5. ¹H NMR (500 MHz, DMSO-d6) δ 10.17 (s, 1H), 8.46 (s, 1H), 8.42 (s, 1H), 8.05 (t, 1H, J=5 Hz), 7.94 (t, 1H, J=4 Hz), 7.91 (dd, 1H, J=9, 2.5 Hz), 7.51 (t, 2H, J=4.5 Hz), 7.46 (m, 1H), 7.20 (m, 1H), 7.13 (m, 2H), 6.94 (d, 1H, J=9 Hz), 3.58 (m, 4H), 3.53 (m, 4H), 2.64 (s, 3H). MS (M+1): 490.3

### Example 43

### N-(2-fluorophenyl)-4-(5-(5-methoxy-3-methylbenzofuran-2-carboxamido)pyridin-2-yl)piperazine-1-carboxamide (43)

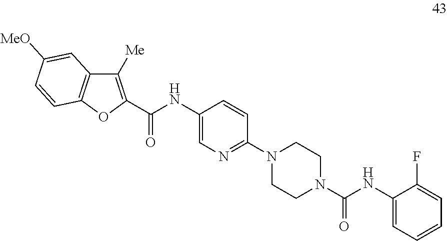

Compound 43 was prepared by the general procedure for compound 1 using intermediate B-5. ¹H NMR (500 MHz, DMSO-d6) δ 10.33 (s, 1H), 8.54 (d, 1H, J=2.5 Hz), 8.42 (s, 1H), 8.00 (dd, 1H, J=9.5, 2.5 Hz), 7.55 (d, 1H, J=9 Hz), 7.45 (m, 1H), 7.27 (d, 1H, J=2.5 Hz), 7.20 (m, 1H), 7.12 (m, 3H), 6.92 (d, 1H, J=9 Hz), 3.85 (s, 3H), 3.57 (m, 4H), 3.53 (m, 4H), 2.58 (s, 3H). MS (M+1): 504.3

### Example 44

### 4-(5-(3-(fluoromethyl)benzofuran-2-carboxamido)pyridin-2-yl)-N-(2-fluorophenyl)piperazine-1-carboxamide (44)

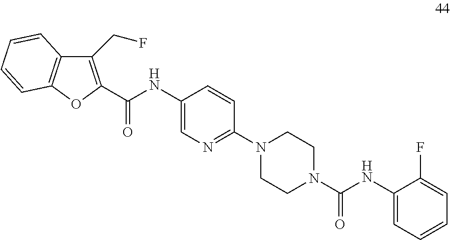

Compound 44 was prepared by the general procedure for compound 1 using intermediates A-16 and B-5. ¹H NMR (500 MHz, DMSO-d6) δ 10.68 (s, 1H), 8.56 (d, 1H, J=2.5 Hz), 8.42 (s, 1H), 8.01 (dd, 1H, J=9.5, 2.5 Hz), 7.90 (d, 1H, J=8 Hz), 7.76 (d, 1H, J=9 Hz), 7.59 (t, 1H, J=7 Hz), 7.45 (m, 2H), 7.20 (m, 1H), 7.13 (m, 2H), 6.94 (d, 1H, J=9 Hz), 6.06 (d, 2H, J=47.5 Hz), 3.57 (m, 4H), 3.55 (m, 4H). MS (M+1): 492.3

### Example 45

### 4-(5-(3-(difluoromethyl)benzofuran-2-carboxamido)pyridin-2-yl)-N-(2-fluorophenyl)piperazine-1-carboxamide (45)

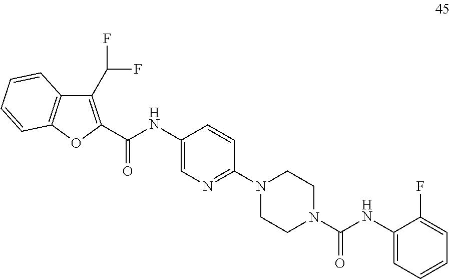

Compound 45 was prepared by the general procedure for compound 1 using intermediates A-19 and B-5. ¹H NMR (500 MHz, DMSO-d6) δ 10.89 (s, 1H), 8.56 (d, 1H, J=2.5 Hz), 8.42 (s, 1H), 8.02 (dd, 1H, J=9, 3 Hz), 7.91 (d, 1H, J=7.5 Hz), 7.82 (t, 1H, J=11.5 Hz), 7.64 (t, 1H, J=8.5 Hz), 7.50 (t, 1H, J=7.5 Hz), 7.45 (m, 2H), 7.20 (m, 1H), 7.13 (m, 2H), 6.95 (d, 1H, J=9 Hz), 3.57 (m, 4H), 3.55 (m, 4H). MS (M+1): 510.3

### Example 46

### N-(2-fluorophenyl)-4-(5-(3-methyl-5-phenylbenzo[b]thiophene-2-carboxamido)pyridin-2-yl)piperazine-1-carboxamide (46)

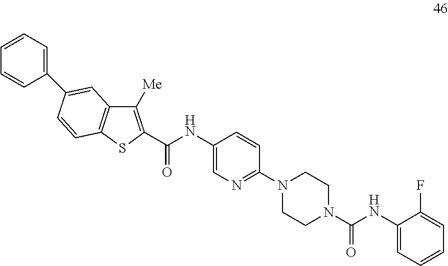

Compound 46 was prepared by the general procedure for compound 1 using intermediates A-8 and B-5. ¹H NMR (500 MHz, DMSO-d6) δ 10.21 (s, 1H), 8.48 (s, 1H), 8.42 (s, 1H), 8.17 (s, 1H), 8.13 (d, 1H, J=8.5 Hz), 7.92 (d, 1H, J=8.5 Hz), 7.82 (d, 3H, J=8 Hz), 7.53 (t, 2H, J=7.5 Hz), 7.46 (m, 1H), 7.42 (t, 1H, J=7.5 Hz), 7.20 (m, 1H), 7.13 (m, 2H), 6.95 (d, 1H, J=9 Hz), 3.57 (m, 4H), 3.54 (m, 4H), 2.71 (s, 3H). MS (M+1): 566.2

### Example 47

### N-(2-fluorophenyl)-4-(5-(3-methyl-5-(pyrimidin-5-yl)benzo[b]thiophene-2-carboxamido)pyridin-2-yl)piperazine-1-carboxamide (47)

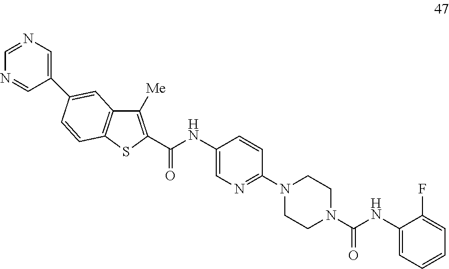

Compound 47 was prepared by the general procedure for compound 1 using intermediates A-9 and B-5. ¹H NMR (500 MHz, DMSO-d6) δ 10.24 (s, 1H), 9.32 (s, 1H), 9.24 (s, 1H), 8.47 (broad s, 1H), 8.40 (d, 2H, J=12.5 Hz), 8.23 (d, 1H, J=8.5 Hz), 7.96 (d, 1H, J=8 Hz), 7.92 (d, 1H, J=9 Hz), 7.46 (m, 1H), 7.20 (m, 1H), 7.13 (m, 2H), 6.95 (d, 1H, J=9 Hz), 3.57 (m, 4H), 3.55 (m, 4H), 2.73 (s, 3H). MS (M+1): 568.2

### Example 48

### methyl 3-((1-(5-(3-methyl-5-phenylbenzo[b]thiophene-2-carboxamido)pyridin-2-yl)piperidin-4-yloxy)methyl)benzoate (48)

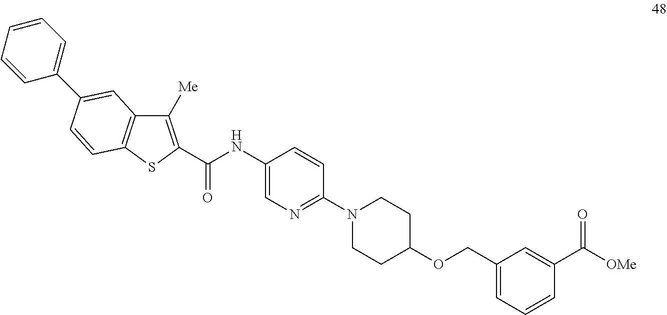

Compound 48 was prepared by the general procedure for compound 1 using intermediates A-8 and B-9. ¹H NMR (500 MHz, CDCl₃) δ 8.25 (d, 1H, J=2.5 Hz), 8.06 (s, 1H), 8.02 (s, 1H), 7.98 (m, 2H), 7.93 (d, 1H, J=8.5 Hz), 7.74 (dd, 1H, J=8, 2 Hz), 7.70 (d, 2H, J=7 Hz), 7.61 (d, 1H, J=7.5 Hz), 7.53 (d, 1H, J=2.5 Hz), 7.51 (d, 2H, J=8 Hz), 7.46 (t, 1H, J=8 Hz), 7.42 (t, 1H, J=7.5 Hz), 6.75 (d, 1H, J=9 Hz), 4.67 (s, 2H), 4.02 (dm, 2H, J=13.5 Hz), 3.95 (s, 3H), 3.69 (m, 1H), 3.27 (td, 2H, J=9.5, 3.5 Hz), 2.83 (s, 3H), 2.04 (m, 2H), 1.76 (qm, 2H, J=8.5 Hz). MS (M+1): 592.2

### Example 49

### methyl 3-((1-(5-(3-methyl-5-phenylbenzo[b]thiophene-2-carboxamido)pyridin-2-yl)piperidin-4-yl)methoxy)benzoate (49)

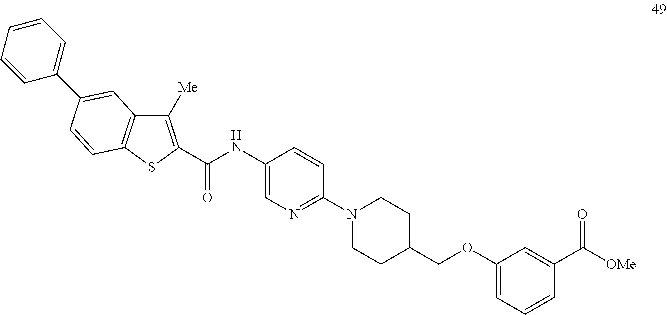

Compound 49 was prepared by the general procedure for compound 1 using intermediates A-8 and B-13. ¹H NMR (500 MHz, CDCl₃) δ 8.25 (d, 1H, 2.5 Hz), 8.02 (s, 1H), 7.98 (d, 1H, J=7 Hz), 7.93 (d, 1H, J=8.5 Hz), 7.74 (dd, 1H, 8.5, 1 Hz), 7.70 (d, 2H, J=8 Hz), 7.66 (d, 1H, J=7.5 Hz), 7.58 (s, 1H), 7.52 (m, 3H), 7.42 (t, 1H, J=8 Hz), 7.37 (t, 1H, J=7.5 Hz), 7.13 (dd, 1H, J=8.5, 2.5 Hz), 6.76 (d, 1H, J=9 Hz), 4.37 (d, 2H, j=13 Hz), 3.94 (s, 3H), 3.91 (d, 2H, J=6 Hz), 2.93 (t, 2H, J=12.5 Hz), 2.83 (s, 3H), 2.11 (m, 1H), 1.98 (d, 2H, J=12 Hz), 1.47 (qd, 2H, J=12.5, 4 Hz). MS (M+1): 592.2

### Example 50

### ethyl 2-(4-(5-(3-methyl-5-phenylbenzo[b]thiophene-2-carboxamido)pyridin-2-yl)piperazine-1-carboxamido)benzoate (50)

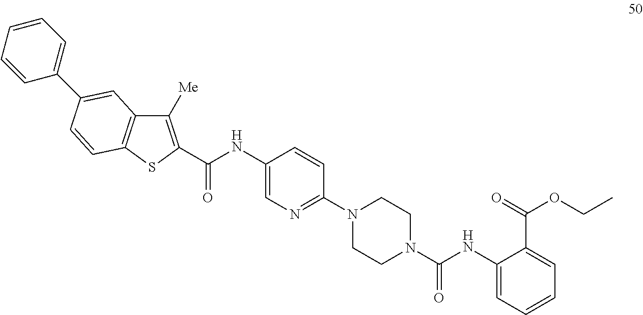

Compound 50 was prepared by the general procedure for compound 1 using intermediates A-8 and B-6. ¹H NMR (500 MHz, DMSO-d6) δ 10.46 (s, 1H), 10.22 (s, 1H), 8.48 (s, 1H), 8.35 (d, 1H, J=8.5 Hz), 8.17 (s, 1H), 8.13 (d, 1H, J=8.5 Hz), 7.96 (d, 1H, J=7.5 Hz), 7.93 (d, 1H, J=8.5 Hz), 7.83 (d, 3H, J=8.5 Hz), 7.58 (t, 1H, J=8 Hz), 7.53 (t, 2H, J=7.5 Hz), 7.42 (t, 1H, J=7.5 Hz), 7.08 (t, 1H, J=7 Hz), 6.95 (d, 1H, J=9.5 Hz), 4.36 (q, 2H, J=8 Hz), 3.61 (m, 8H), 2.71 (s, 3H), 1.35 (t, 3H, J=6.5 Hz). MS (M+Na): 642.2

### Example 51

### 3-((1-(5-(3-methyl-5-phenylbenzo[b]thiophene-2-carboxamido)pyridin-2-yl)piperidin-4-yloxy)methyl)benzoic acid (51)

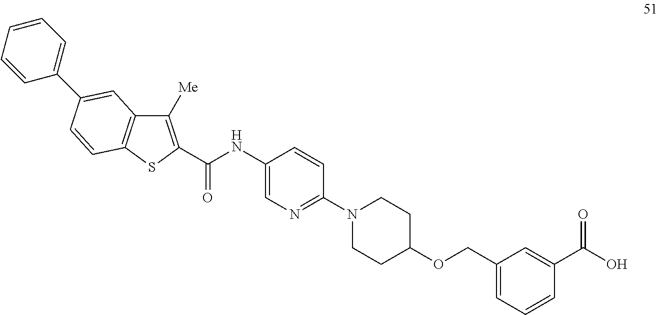

Compound 51 was prepared by the hydrolysis of compound 48.

¹H NMR (500 MHz, DMSO-d6) δ 10.17 (s, 1H), 8.42 (s, 1H), 8.17 (s, 1H), 8.13 (d, 1H, J=8.5 Hz), 7.94 (s, 1H), 7.86 (d, 2H, J=8 Hz), 7.82 (d, 3H, J=8 Hz), 7.58 (d, 1H, J=7 Hz), 7.52 (t, 2H, J=7.5 Hz), 7.47 (t, 1H, J=7.5 Hz), 7.41 (t, 1H, J=8 Hz), 6.91 (d, 1H, J=9 Hz), 4.63 (s, 2H), 3.96 (m, 2H), 3.67 (m, 1H), 3.17 (t, 2H, J=10 Hz), 2.70 (s, 3H), 1.96 (m, 2H), 1.53 (m, 2H). MS (M+Na): 578.2

### Example 52

### 3-((1-(5-(3-methyl-5-phenylbenzo[b]thiophene-2-carboxamido)pyridin-2-yl)piperidin-4-yl)methoxy)benzoic acid (52)

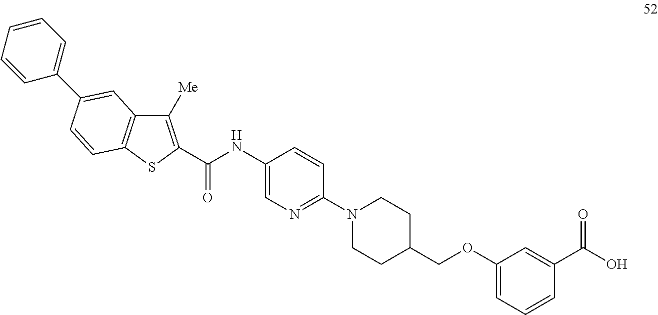

Compound 52 was prepared by the hydrolysis of compound 49. ¹H NMR (500 MHz, DMSO-d6) δ 10.16 (s, 1H), 8.42 (s, 1H), 8.17 (s, 1H), 8.13 (d, 1H, J=8.5 Hz), 7.86 (d, 2H, J=9.5 Hz), 7.82 (d, 3H, J=8 Hz), 7.52 (m, 3H), 7.45 (s, 1H), 7.42 (d, 1H, J=8 Hz), 7.39 (d, 1H, J=8 Hz), 7.19 (d, 1H, J=8 Hz), 6.90 (d, 1H, J=9 Hz), 4.32 (d, 2H, J=13 Hz), 3.92 (d, 2H, J=6.5 Hz), 2.84 (t, 2H, J=12 Hz), 2.71 (s, 3H), 2.04 (m, 1H), 1.86 (d, 2H, J=13 Hz), 1.32 (q, 2H, J=12.5 Hz). MS (M+Na): 578.2

### Example 53

### 2-(4-(5-(3-methyl-5-phenylbenzo[b]thiophene-2-carboxamido)pyridin-2-yl)piperazine-1-carboxamido)benzoic acid (53)

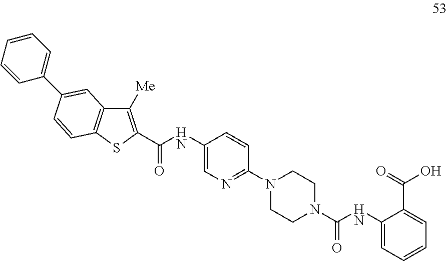

Compound 53 was prepared by the hydrolysis of compound 50.

¹H NMR (500 MHz, DMSO-d6) δ 11.08 (broad s, 1H), 10.22 (s, 1H), 8.48 (s, 1H), 8.44 (d, 1H, J=8.5 Hz), 8.17 (s, 1H), 8.13 (d, 1H, J=8.5 Hz), 7.97 (d, 1H, J=8 Hz), 7.92 (d, 1H, J=8.5 Hz), 7.82 (d, 3H, J=8 Hz), 7.53 (m, 3H), 7.42 (t, 1H, J=7.5 Hz), 7.03 (t, 1H, J=8 Hz), 6.94 (d, 1H, J=9 Hz), 3.62 (m, 4H), 3.60 (m, 4H), 2.71 (s, 3H). MS (M+1): 592.2

### Example 54

### N-(2-fluorophenyl)-4-(5-(5-(3-fluorophenyl)-3-methylbenzo[b]thiophene-2-carboxamido)pyridin-2-yl)piperazine-1-carboxamide (54)

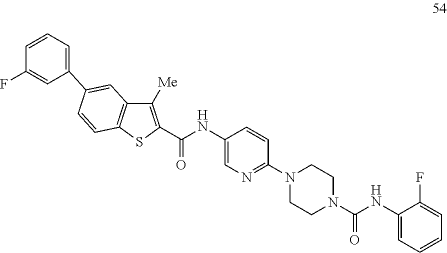

Compound 54 was prepared by the general procedure for compound 1 using intermediates A-11 and B-5. ¹H NMR (500 MHz, DMSO-d6) δ 10.22 (s, 1H), 8.47 (s, 1H), 8.42 (s, 1H), 8.24 (s, 1H), 8.15 (d, 1H, J=8.5 Hz), 7.92 (d, 1H, J=9 Hz), 7.86 (d, 1H, J=8.5 Hz), 7.72 (d, 1H, J=11 Hz), 7.70 (d, 1H, J=9 Hz), 7.56 (q, 1H, J=6.5 Hz), 7.46 (m, 1H), 7.24 (t, 1H, J=9 Hz), 7.20 (m, 1H), 7.13 (m, 2H), 6.95 (d, 1H, J=9 Hz), 3.57 (m, 4H), 3.54 (m, 4H), 2.72 (s, 3H). MS (M+1): 584.2

### Example 55

### N-(2-fluorophenyl)-4-(5-(5-(4-fluorophenyl)-3-methylbenzo[b]thiophene-2-carboxamido)pyridin-2-yl)piperazine-1-carboxamide (55)

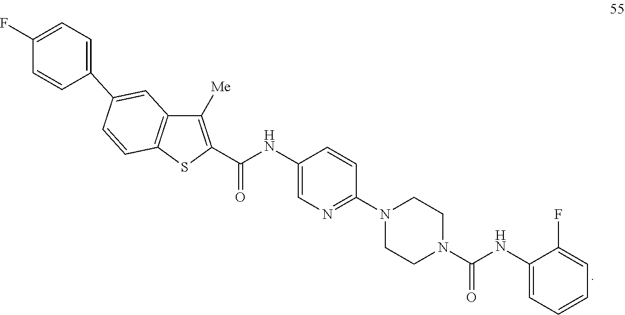

Compound 55 was prepared by the general procedure for compound 1 using intermediates A-12 and B-5. ¹H NMR (500 MHz, DMSO-d6) δ 10.21 (s, 1H), 8.47 (s, 1H), 8.42 (s, 1H), 8.16 (s, 1H), 8.13 (d, 1H, J=8.5 Hz), 7.92 (d, 1H, J=8 Hz), 7.88 (d, 1H, J=5.5 Hz), 7.87 (d, 1H, J=5.5 Hz), 7.81 (d, 1H, J=8 Hz), 7.46 (m, 1H), 7.35 (t, 2H, J=7.5 Hz), 7.20 (m, 1H), 7.13 (m, 2H), 6.95 (d, 1H, J=9 Hz), 3.57 (m, 4H), 3.54 (m, 4H), 2.71 (s, 3H). MS (M+1): 584.1

### Example 56

### methyl 3-((1-(5-(3-methyl-5-phenylbenzofuran-2-carboxamido)pyrimidin-2-yl)piperidin-4-yl)methoxy)benzoate (56)

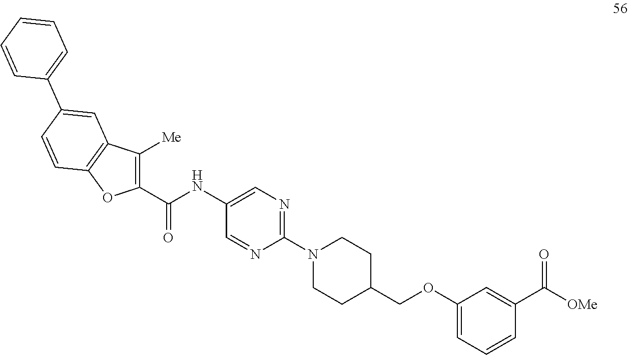

To intermediate B-25 (50.0 mg, 0.15 mmol) in dry DMF (1.73 mL) was added 3-methyl-5-phenylbenzofuran-2-carboxylic acid A-3 (47.9 mg, 0.19 mmol), HATU (111.0 mg, 0.29 mmol), and N,N-diisopropylethylamine (0.08 mL, 0.44 mmol). The reaction mixture was stirred at RT for 48 h then concentrated. Water was added, and the aqueous solution was extracted with ethyl acetate. The combined organic extract was successively washed with water then brine, dried (MgSO₄), filtered, and concentrated. Purification by C₁₈ reverse phase chromatography (eluant: 10-100% CH₃CN in water-with 0.1% HCOOH) yielded compound 56 as a light beige solid (98.7 mg, 95% yield). ¹H NMR (500 MHz, CDCl₃) δ 8.65 (s, 2H), 8.08 (broad s, 1H), 7.84 (d, 1H, J=1.5 Hz), 7.72 (dd, 1H, J=1.5, 8.5 Hz), 7.65-7.68 (m, 3H), 7.60-7.58 (m, 2H), 7.51 (t, 2H, J=7.5 Hz), 7.41 (t, 1H, J=7.0 Hz), 7.37 (t, 1H, J=8.5 Hz), 7.13 (dd, 1H, J=2.5, 8.5 Hz), 4.85 (broad d, 2H, J=13.5 Hz), 3.91 (d, 2H, J=6.5 Hz), 3.00 (dt, 2H, J=2.5, 13.0 Hz), 2.83 (s, 3H), 2.74 (s, 3H), 2.15 (m, 1H), 1.99 (broad d, 2H, J=12.0 Hz), 1.41 (dq, 211, J=4.5, 13.0 Hz). MS (M+1): 577.2.

### Example 57

### 3-((1-(5-(3-methyl-5-phenylbenzofuran-2-carboxamido)pyrimidin-2-yl)piperidin-4-yl)methoxy)benzoic acid (57)

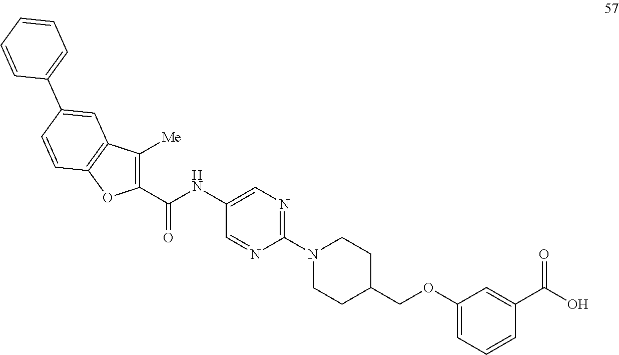

To a stirred solution of compound 56 (88.0 mg, 0.13 mmol) in MeOH (0.86 mL), THF (0.86 mL), and water (0.86 mL) was added lithium hydroxide monohydrate (28.0 mg, 0.66 mmol). The reaction mixture was stirred at RT for 16 h, then heated to 65° C. for 90 min under microwave irradiation. 1 N HCl (5.0 mL) was then added, and the solution was concentrated. The solid residue was triturated in water (10 mL), filtered, then successively rinsed with water (2×10 mL), diethyl ether (2×5 mL) and dried under high vacuum to yield compound 57 as a white solid (62.0 mg, 84% yield). ¹H NMR (500 MHz, DMSO-d₆) δ 13.00 (broad s, 1H), 10.47 (s, 1H), 8.73 (s, 2H), 8.06 (s, 1H), 7.81 (d, 1H, J=9.0 Hz), 7.76 (d, 2H, J=8.0 Hz), 7.73 (d, 1H, J=8.5 Hz), 7.54-7.49 (m, 3H), 7.45 (broad s, 1H), 7.43-7.38 (m, 2H), 7.20 (broad d, 1H, J=8.5 Hz), 4.69 (broad d, 2H, J=13.0 Hz), 3.93 (d, 2H, J=6.5 Hz), 2.95 (t, 2H, J=12.5 Hz), 2.65 (s, 3H), 2.10 (m, 1H), 1.87 (broad d, 2H, J=11.0 Hz), 1.28 (dq, 2H, J=3.5, 12.5 Hz). MS (M+1): 563.2.

### Example 58

### 2,2-dim ethyl-3-(5-(4-(3-methyl-5-phenylbenzofuran-2-carboxamido)phenyl)pyridin-2-yloxy)propanoic acid (58)

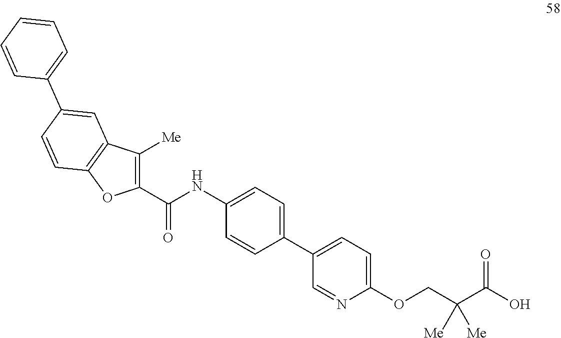

Compound 58 was prepared by the general procedure for compound 56 using intermediates A-3 and B-19. ¹H NMR (500 MHz, DMSO-d₆) δ10.57 (s, 1H), 8.47 (d, 1H, J=2.5 Hz), 8.07 (s, 1H), 8.00 (dd, 1H, J=2.0, 8.5 Hz), 7.97 (d, 2H, J=8.5 Hz), 7.83-7.76 (m, 4H), 7.68 (d, 2H, J=8.5 Hz), 7.51 (t, 2H, J=7.5 Hz), 7.40 (t, 1H, J=7.5 Hz), 6.85 (d, 1H, J=8.5 Hz), 4.26 (s, 2H), 2.68 (s, 3H), 1.15 (s, 6H). MS (M+1): 521.2.

### Example 59

### 2,2-dimethyl-3-(5-(3-methyl-5-phenylbenzofuran-2-carboxamido)-2,3′-bipyridin-6′-yloxy)propanoic acid (59)

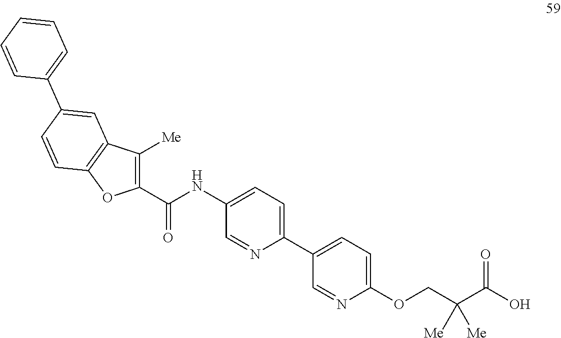

Compound 59 was prepared by the general procedure for compound 56 using intermediates A-3 and B-28. ¹H NMR (500 MHz, DMSO-d₆) δ 12.39 (broad s, 1H), 10.81 (s, 1H), 9.11 (d, 1H, J=2.5 Hz), 8.86 (d, 1H, J=2.5 Hz), 8.37 (m, 2H), 8.09 (s, 1H), 7.99 (d, 1H, J=9.0 Hz), 7.84 (m, 1H), 7.78 (m, 3H), 7.52 (t, 2H, J=8.0 Hz), 7.40 (t, 1H, J=7.5 Hz), 6.92 (d, 1H, J=8.5 Hz), 4.32 (s, 2H), 2.69 (s, 3H), 1.24 (s, 6H). MS (M+1): 522.2.

### Example 60

### methyl 3-((1-(2-fluoro-4-(3-methyl-5-phenylbenzofuran-2-carboxamido)phenyl)piperidin-4-yl)methoxy)benzoate (60)

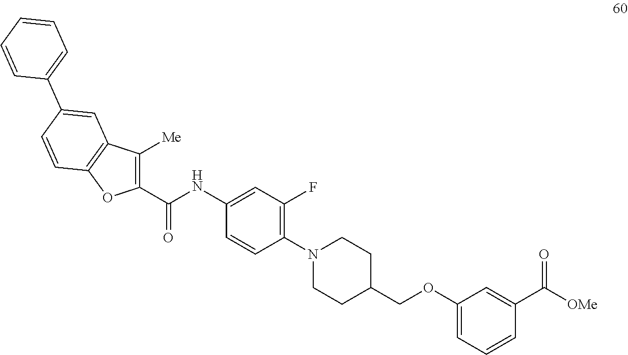

Compound 60 was prepared by the general procedure for compound 56 using intermediates A-3 and B-29. ¹H NMR (500 MHz, DMSO-d₆) δ 10.50 (s, 1H), 8.06 (s, 1H), 7.83-7.74 (m, 5H), 7.59 (broad d, 1H, J=8.0 Hz), 7.56 (d, 1H, J=7.0 Hz), 7.51 (t, 2H, J=7.5 Hz), 7.44-7.48 (m, 2H), 7.40 (t, 1H, J=7.5 Hz), 7.27 (broad d, 1H, J=8.5 Hz), 7.07 (t, 1H, J=9.5 Hz), 3.97 (d, 2H, J=5.5 Hz), 3.86 (s, 3H), 3.36-3.40 (m, 2H), 2.70 (t, 2H, J=10.5 Hz), 2.65 (s, 3H), 1.91 (m, 3H), 1.51 (m, 2H). MS (M+1): 593.2.

### Example 61

### methyl 3-((1-(4-(3-methyl-5-phenylbenzofuran-2-carboxamido)phenyl)piperidin-4-yl)methoxy)benzoate (61)

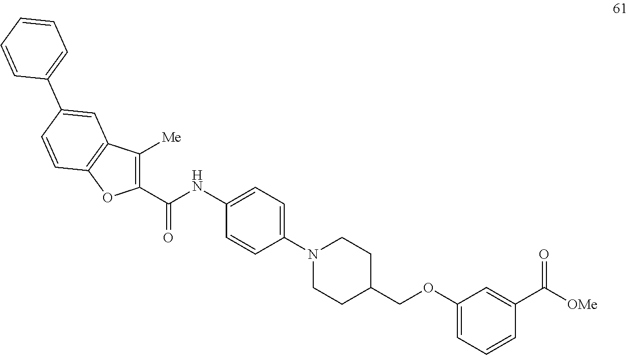

Compound 61 was prepared by the general procedure for compound 56 using intermediates A-3 and B-27. ¹H NMR (500 MHz, DMSO-d₆) δ 10.25 (s, 1H), 8.04 (broad s, 1H), 7.79 (dd, 1H, J=2.0, 9.0 Hz), 7.77 (d, 2H, J=8.0 Hz), 7.73 (d, 1H, J=8.5 Hz), 7.68 (d, 2H, J=9.0 Hz), 7.55 (d, 1H, J=8.0 Hz), 7.51 (t, 2H, J=7.5 Hz), 7.44-7.48 (m, 2H), 7.40 (t, 1H, J=7.0 Hz), 7.26 (dd, 1H, J=2.5, 8.0 Hz), 6.96 (d, 2H, J=9.0 Hz), 3.96 (d, 2H, 6.0 Hz), 3.86 (s, 3H), 3.72 (broad d, 2H, J=11.5 Hz), 2.69 (1, 2H, J=11.0 Hz), 2.65 (s, 3H), 1.89 (m, 3H), 1.40 (m, 2H). MS (M+1): 575.3.

### Example 62

### 3-((1-(2-fluoro-4-(3-methyl-5-phenylbenzofuran-2-carboxamido)phenyl)piperidin-4-yl)methoxy)benzoic acid (62)

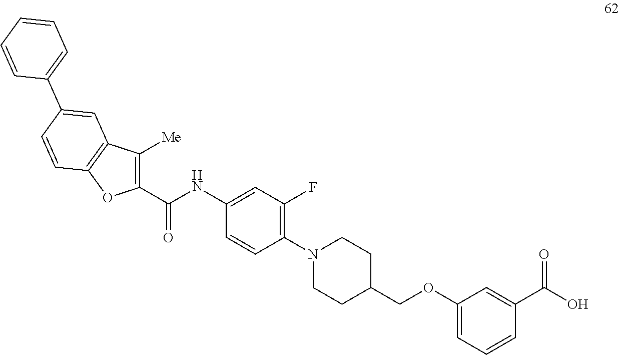

Compound 62 was prepared by the general procedure for compound 56 using intermediates A-3 and B-29. ¹H NMR (500 MHz, DMSO-d₆) δ 10.49 (s, 1H), 8.06 (s, 1H), 7.81 (dd, 1H, J=2.0, 8.5 Hz), 7.74-7.77 (m, 4H), 7.59 (broad d, 1H, J=9.0 Hz), 7.49-7.53 (m, 3H), 7.46 (broad s, 1H), 7.39 (m, 2H), 7.17 (broad d, 1H, J=8.5 Hz), 7.07 (t, 1H, J=9.5 Hz), 3.95 (broad d, 2H, J=6.0 Hz), 3.37 (m, 2H), 2.70 (t, 211, J=10.5 Hz), 2.66 (s, 3H), 1.90 (m, 3H), 1.51 (q, 2H, J=12.0 Hz). MS (M+1): 579.2.

### Example 63

### 3-((1-(4-(3-methyl-5-phenylbenzofuran-2-carboxamido)phenyl)piperidin-4-yl)methoxy)benzoic acid (63)

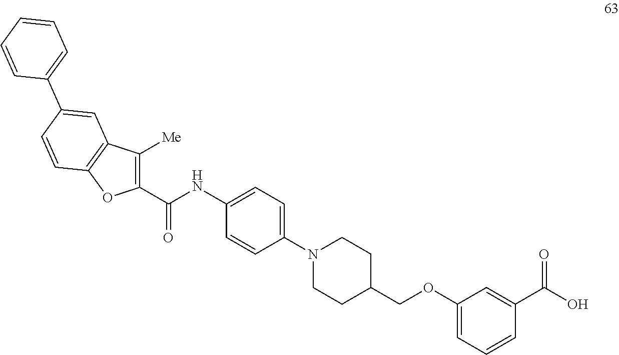

Compound 63 was prepared by the general procedure for compound 56 using intermediates A-3 and B-27. ¹H NMR (500 MHz, DMSO-d₆) δ 10.23 (s, 1H), 8.38 (s, 1H), 8.05 (s, 1H), 7.72-7.81 (m, 4H), 7.68 (d, 2H, J=9.0 Hz), 7.48-7.52 (m, 2H), 7.45 (broad s, 1H), 7.40 (t, 1H, J=7.0 Hz), 7.32 (t, 1H, J=8.5 Hz), 7.08 (m, 1H), 6.96 (d, 2H, J=9.0 Hz), 3.92 (d, 2H, J=5.5 Hz), 3.72 (broad d, 2H, J=11.5 Hz), 2.69 (t, 211, J=12.0 Hz), 2.65 (s, 3H), 1.90 (m, 3H), 1.45 (m, 2H). MS (M+1): 561.2.

### Example 64

### 3-((1-(5-(5-(4-methoxyphenyl)-3-methylbenzofuran-2-carboxamido)pyridin-2-yl)piperidin-4-yloxy)methyl)benzoic acid (64)

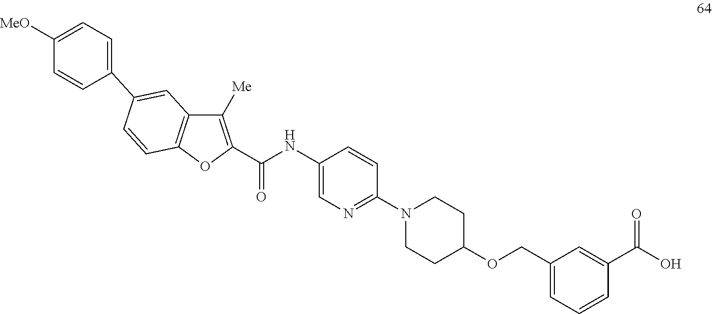

Compound 64 was prepared by the general procedure for compound 1 and subsequent hydrolysis. ¹H NMR (500 MHz, DMSO-d6) δ 10.53 (broad s, 1H), 8.55 (s, 1H), 8.11 (broad s, 1H), 7.99 (s, 1H), 7.95 (s, 1H), 7.87 (d, 1H, J=7.5 Hz), 7.76 (d, 1H, J=9 Hz), 7.70 (m, 3H), 7.61 (d, 1H, J=7.5 Hz), 7.50 (t, 1H, J=7.5 Hz), 7.16 (broad s, 1H), 7.06 (d, 2H, J=8 Hz), 4.65 (s, 2H), 3.94 (m, 2H), 3.82 (s, 3H), 3.71 (m, 1H), 3.32 (m, 2H), 2.65 (s, 3H), 1.99 (m, 2H), 1.60 (m, 2H). MS (M+1): 592.5

### Example 65

### ethyl 2-(4-(5-(5-(4-methoxyphenyl)-3-methylbenzofuran-2-carboxamido)pyridin-2-yl)piperazine-1-carboxamido)benzoate (65)

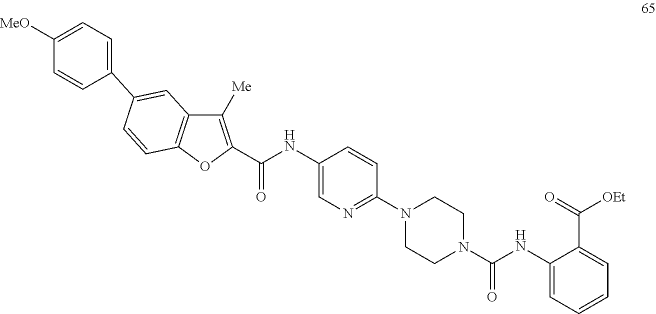

Compound 65 was prepared by the general procedure for compound 1. ¹H NMR (500 MHz, DMSO-d6) δ 10.46 (s, 1H), 10.39 (s, 1H), 8.57 (d, 1H, J=2.5 Hz), 8.35 (d, 1H, J=8.5 Hz), 8.02 (dd, 1H, J=9, 2.5 Hz), 7.98 (s, 1H), 7.96 (d, 1H, J=8.5 Hz), 7.75 (d, 1H, J=9 Hz), 7.70 (d, 3H, J=9 Hz), 7.58 (t, 1H, J=7 Hz), 7.07 (m, 3H), 6.93 (d, 1H, J=9 Hz), 4.36 (q, 2H, J=7 Hz), 3.82 (s, 3H), 3.61 (m, 8H), 2.64 (s, 3H), 1.35 (t, 3H, J=7 Hz). MS (M+1): 634.5

### Example 66

### ethyl 2-(4-(5-(5-(4-methoxyphenyl)-3-methylbenzofuran-2-carboxamido)pyridin-2-yl)piperazine-1-carboxamido)benzoate (66)

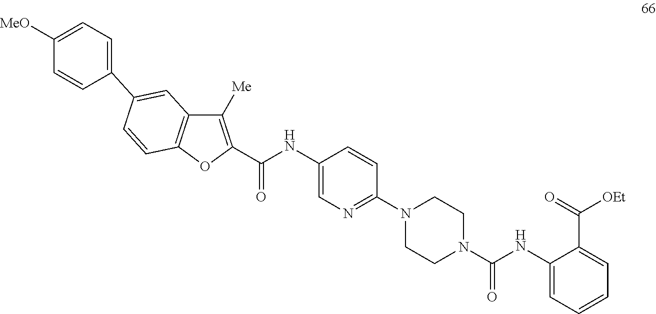

Compound 66 was prepared by the general procedure for compound 1. ¹H NMR (500 MHz, DMSO-d6) δ 10.97 (s, 1H), 10.68 (broad s, 1H), 8.65 (s, 1H), 8.44 (d, 1H, J=8.5 Hz), 8.26 (d, 1H, J=9 Hz), 8.00 (s, 1H), 7.98 (d, 1H, j=7.5 Hz), 7.78 (d, 1H, J=9 Hz), 7.71 (t, 3H, J=8.5 Hz), 7.56 (t, 1H, J=8.5 Hz), 7.30 (broad s, 1H), 7.05 (m, 3H), 3.82 (s, 3H), 3.77 (broad s, 4H), 3.70 (broad s, 4H), 2.66 (s, 3H). MS (M+1): 606.4

### Example 67

### methyl 3-((1-(5-(5-(2-methoxyphenyl)-3-methylbenzofuran-2-carboxamido)pyridin-2-yl)piperidin-4-yl)methoxy)benzoate (67)

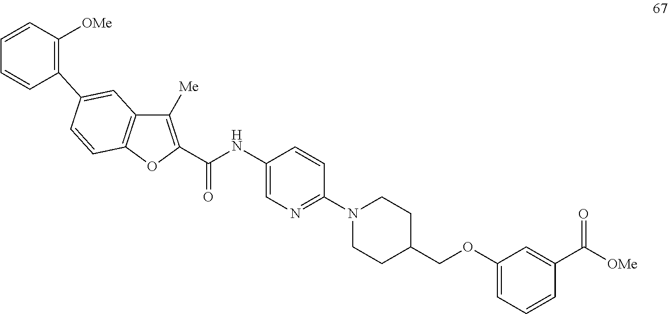

Compound 67 was prepared by the general procedure for compound 1. ¹H NMR (500 MHz, DMSO-d6) δ 10.34 (s, 1H), 8.52 (d, 1H, J=2.5 Hz), 7.96 (dd, 1H, J=9, 2.5 Hz), 7.80 (s, 1H), 7.67 (d, 1H, J=8.5 Hz), 7.61 (d, 1H, J=9 Hz), 7.55 (d, 1H, J=8 Hz), 7.46 (m, 2H), 7.38 (m, 2H), 7.25 (dd, 1H, J=8.5, 2.5 Hz), 7.15 (d, 1H, J=8.5 Hz), 7.07 (t, 1H, J=8 Hz), 6.88 (d, 1H, J=9 Hz), 4.32 (d, 2H, J=13 Hz), 3.93 (d, 2H, J=6.5 Hz), 3.86 (s, 3H), 3.79 (s, 3H), 2.83 (t, 2H, J=11.5 Hz), 2.61 (s, 3H), 2.03 (m, 1H), 1.86 (d, 2H, J=11.5 Hz), 1.33 (qd, 2H, J=12.5, 3.5 Hz). MS (M+1): 606.5

### Example 68

### ethyl 2-(4-(5-(5-(2-methoxyphenyl)-3-methylbenzofuran-2-carboxamido)pyridin-2-yl)piperazine-1-carboxamido)benzoate (68)

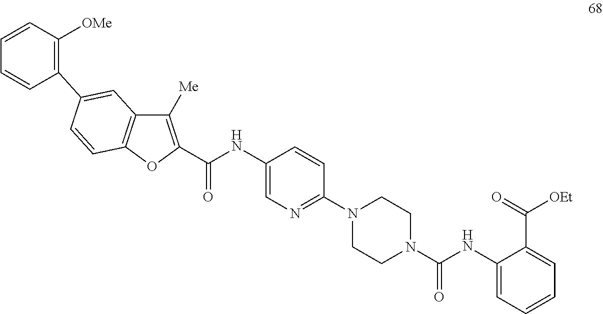

Compound 68 was prepared by the general procedure for compound 1. ¹H NMR (500 MHz, DMSO-d6) δ 10.46 (s, 1H), 10.40 (s, 1H), 8.58 (d, 1H, J=2.5 Hz), 8.35 (d, 1H, J=8 Hz), 8.03 (dd, 1H, J=9, 2.5 Hz), 7.96 (d, 1H, J=7.5 Hz), 7.81 (s, 1H), 7.68 (d, 1H, J=8.5 Hz), 7.61 (d, 1H, J=8.5 Hz), 7.58 (t, 1H, 8.5 Hz), 7.38 (m, 2H), 7.15 (d, 1H, J=8.5 Hz), 7.07 (td, 2H, J=8, 2 Hz), 6.93 (d, 1H, J=9.5 Hz), 4.36 (q, 2H, J=7 Hz), 3.79 (s, 3H), 3.61 (m, 8H), 2.61 (s, 3H), 1.35 (t, 3H, J=7 Hz). MS (M+1): 634.5

### Example 69

### N-(2-fluorophenyl)4-(5-(5-(2-methoxyphenyl)-3-methylbenzofuran-2-carboxamido)pyridin-2-yl)piperazine-1-carboxamide (69)

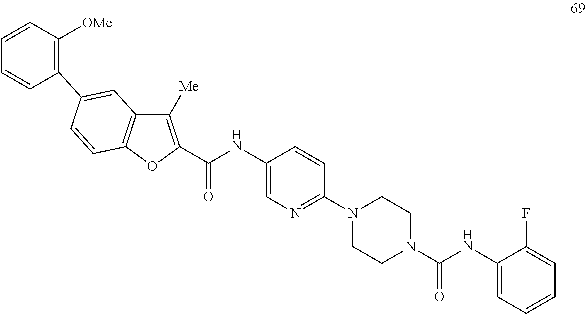

Compound 69 was prepared by the general procedure for compound 1. ¹H NMR (500 MHz, DMSO-d6) δ 10.41 (s, 1H), 8.58 (d, 1H, J=2.5 Hz), 8.43 (s, 1H), 8.03 (dd, 1H, J=9, 2.5 Hz), 7.81 (s, 1H), 7.68 (d, 1H, J=8.5 Hz), 7.62 (dd, 1H, J=8.5, 2 Hz), 7.46 (s, 1H), 7.38 (d, 2H, J=7 Hz), 7.21 (m, 1H), 7.14 (m, 3H), 7.07 (t, 1H, J=7 Hz), 6.94 (d, 1H, J=9 Hz), 3.79 (s, 3H), 3.58 (m, 4H), 3.54 (m, 4H), 2.62 (s, 3H). MS (M+1): 580.5

### Example 70

### ethyl 2-(4-(5-(5-(2-methoxyphenyl)-3-methylbenzofuran-2-carboxamido)pyridin-2-yl)piperazine-1-carboxamido)benzoate (70)

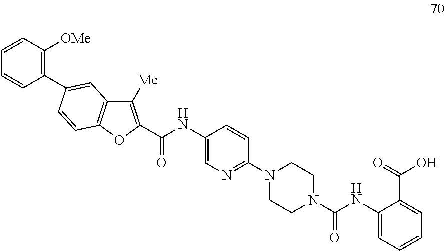

Compound 70 was prepared by the general procedure for compound 1 and subsequent hydrolysis. ¹H NMR (500 MHz, DMSO-d6) δ 10.97 (s, 1H), 10.77 (s, 1H), 8.69 (s, 1H), 8.44 (d, 1H, J=8.5 Hz), 8.32 (d, 1H, J=9 Hz), 7.98 (d, 1H, J=8 Hz), 7.83 (s, 1H), 7.69 (d, 1H, J=8.5 Hz), 7.63 (d, 1H, J=9 Hz), 7.56 (t, 1H, J=7.5 Hz), 7.38 (m, 3H), 7.15 (d, 1H, J=8.5 Hz), 7.05 (m, 2H), 3.82 (m, 4H), 3.79 (s, 3H), 3.72 (m, 4H), 2.63 (s, 3H). MS (M+1-1): 606.4

### Example 71

### methyl 3-((1-(5-(5-(2-methoxyphenyl)-3-methylbenzofuran-2-carboxamido)pyridin-2-yl)piperidin-4-yl)methoxy)benzoate (71)

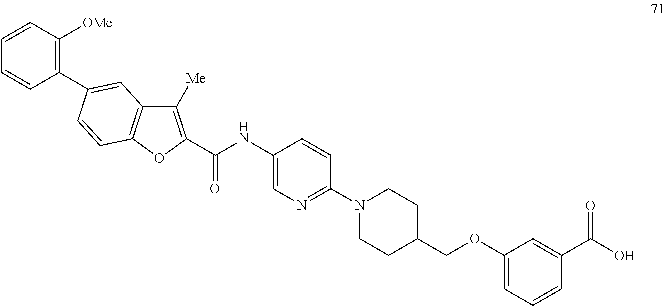

Compound 71 was prepared by the general procedure for compound 1 and subsequent hydrolysis. ¹H NMR (500 MHz, DMSO-d6) δ 10.72 (broad s, 1H), 8.63 (s, 1H), 8.26 (broad s, 1H), 7.83 (s, 1H), 7.69 (d, 1H, J=8.5 Hz), 7.64 (d, 1H, J=9 Hz), 7.54 (d, 1H, J=7.5 Hz), 7.46 (s, 1H), 7.42 (t, 2H, J=8 Hz), 7.38 (d, 2H, J=7.5 Hz), 7.22 (dd, 1H, 1H, J=8.5, 2.5 Hz), 7.16 (d, 1H, J=7.5 Hz), 7.07 (t, 1H, J=7 Hz), 4.31 (d, 2H, J=13 Hz), 3.95 (d, 2H, J=6.5 Hz), 3.79 (s, 3H), 3.17 (m, 2H), 2.63 (s, 3H), 2.15 (m, 1H), 1.94 (d, 2H, J=11 Hz), 1.43 (q, 2H, J=11 Hz). MS (M+1): 592.5

### Example 72

### methyl 3-((1-(5-(5-(2-methoxyphenyl)-3-methylbenzofuran-2-carboxamido)pyridin-2-yl)piperidin-4-yloxy)methyl)benzoate (72)

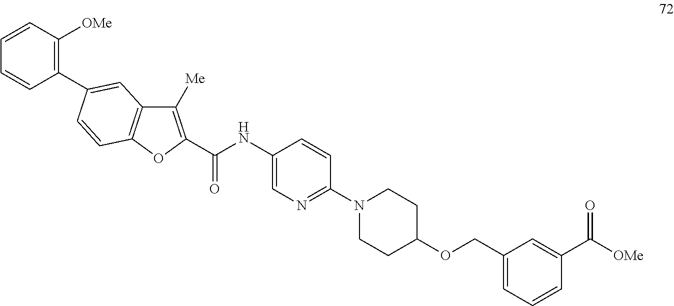

Compound 72 was prepared by the general procedure for compound 1. ¹H NMR (500 MHz, DMSO-d6) δ 10.36 (s, 1H), 8.52 (d, 1H, J=2.5 Hz), 7.97 (d, 1H, J=9.5 Hz), 7.96 (s, 1H), 7.88 (d, 1H, J=7.5 Hz), 7.80 (s, 1H), 7.67 (d, 1H, J=8.5 Hz), 7.65 (d, 1H, J=8 Hz), 7.61 (d, 1H, J=8.5 Hz), 7.52 (t, 1H, J=7.5 Hz), 7.38 (m, 2H), 7.15 (d, 1H, J=8.5 Hz), 7.07 (t, 1H, J=7.5 Hz), 6.90 (d, 1H, J=9 Hz), 4.65 (s, 2H), 3.97 (m, 2H), 3.87 (s, 3H), 3.79 (s, 3H), 3.67 (m, 1H), 3.15 (t, 2H, J=10.5 Hz), 2.61 (s, 3H), 1.97 (m, 2H), 1.53 (q, 211, J=9.5 Hz). MS (M+1): 606.2

### Example 73

### 3-((1-(5-(5-(2-methoxyphenyl)-3-methylbenzofuran-2-carboxamido)pyridin-2-yl)piperidin-4-yloxy)methyl)benzoic acid (73)

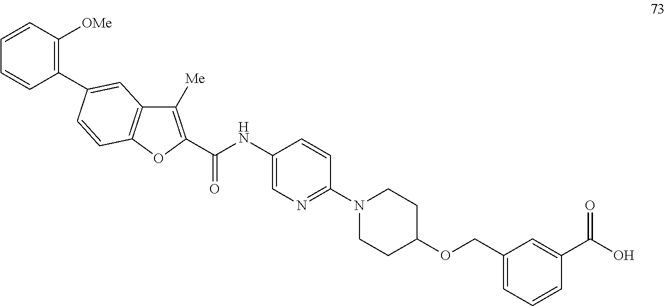

Compound 73 was prepared by the general procedure for compound 1 and subsequent hydrolysis. ¹H NMR (500 MHz, DMSO-d6) δ 10.70 (broad s, 1H), 8.63 (s, 1H), 8.25 (broad s, 1H), 7.95 (s, 1H), 7.87 (d, 1H, J=7.5 Hz), 7.82 (s, 1H), 7.69 (d, 1H, J=8.5 Hz), 7.63 (m, 2H), 7.50 (t, 1H, J=8 Hz), 7.38 (m, 3H), 7.15 (d, 1H, J=8 Hz), 7.07 (t, 1H, J=7 Hz), 4.65 (s, 2H), 3.96 (m, 2H), 3.79 (s, 3H), 3.75 (m, 1H), 3.46 (m, 2H), 2.62 (s, 3H), 2.01 (m, 2H), 1.66 (m, 2H). MS (M+1): 592.1

### Example 74

### 4-((1-(5-(3-methyl-5-phenylbenzofuran-2-carboxamido)pyridin-2-yl)piperidin-4-yl)methoxy)benzoic acid (74)

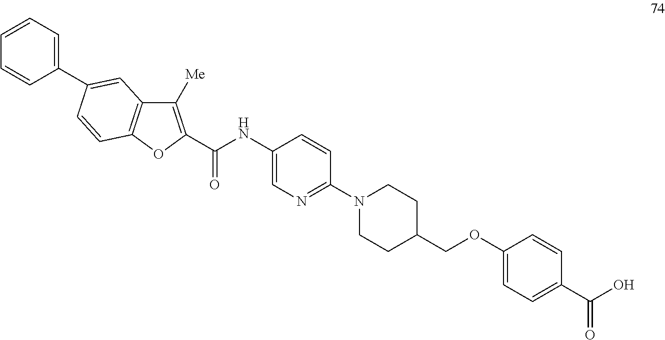

Compound 74 was prepared by the general procedure for compound 1 and subsequent hydrolysis. ¹H NMR (500 MHz, DMSO-d6) δ 10.38 (s, 1H), 8.51 (d, 1H, J=2 Hz), 8.05 (s, 1H), 7.96 (dd, 1H, J=9, 2.5 Hz), 7.77 (m, 6H), 7.51 (t, 2H, J=8 Hz), 7.40 (t, 1H, J=7.5 Hz), 6.88 (d, 1H, J=9 Hz), 6.82 (d, 2H, J=8 Hz), 4.32 (d, 2H, J=13 Hz), 3.87 (d, 2H, J=6.5 Hz), 2.82 (t, 2H, J=12 Hz), 2.65 (s, 3H), 2.02 (m, 1H), 1.85 (d, 2H, J=13 Hz), 1.31 (q, 2H, J=12 Hz). MS (M+1): 562.3

### Example 75

### 2-((1-(5-(3-methyl-5-phenylbenzofuran-2-carboxamido)pyridin-2-yl)piperidin-4-yl)methoxy)benzoic acid (75)

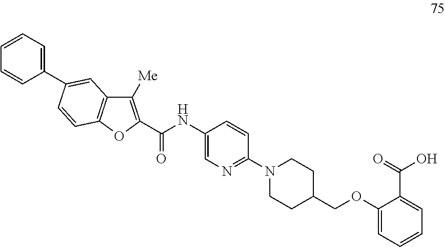

Compound 75 was prepared by the general procedure for compound 1 and subsequent hydrolysis. ¹H NMR (500 MHz, DMSO-d6) δ 10.78 (s, 1H), 8.63 (s, 1H), 8.31 (d, 1H, J=9.5 Hz), 8.08 (s, 1H), 7.83 (d, 1H, J=8.5 Hz), 7.76 (m, 3H), 7.63 (d, 1H, J=8 Hz), 7.50 (m, 4H), 7.40 (t, 1H, J=7 Hz), 7.12 (d, 1H, J=8.5 Hz), 7.00 (t, 1H, J=7.5 Hz), 4.29 (d, 2H, J=13 Hz), 3.96 (d, 2H, J=6 Hz), 3.22 (t, 2H, J=11.5 Hz), 2.67 (s, 3H), 2.16 (m, 1H), 1.97 (d, 2H, J=12 Hz), 1.46 (q, 2H, J=10.5 Hz). MS (M+1): 562.3

### Example 76

### 3-((1-(5-(5-(4-methoxyphenyl)-3-methylbenzofuran-2-carboxamido)pyridin-2-yl)piperidin-4-yloxy)methyl)benzoic acid (76)

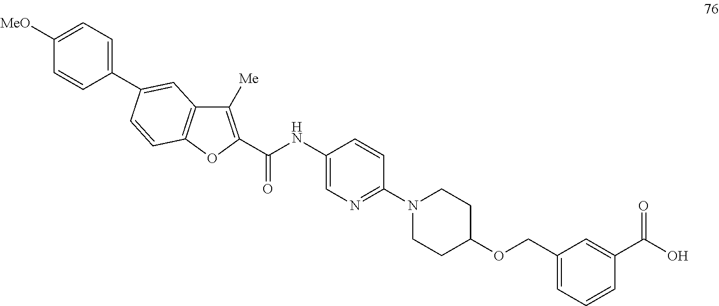

Compound 76 was prepared by the general procedure for compound 1 and subsequent hydrolysis. ¹H NMR (500 MHz, DMSO-d6) δ 13.10 (broad s, 1H), 10.37 (s, 1H), 8.52 (s, 1H), 8.05 (s, 1H), 7.97 (d, 1H, J=9.5 Hz), 7.80 (d, 1H, J=8.5 Hz), 7.76 (m, 3H), 7.65 (d, 1H, J=8.5 Hz), 7.56 (m, 1H), 7.51 (t, 2H, J=7.5 Hz), 7.40 (t, 1H, J=7 Hz), 7.34 (t, 1H, J=8.5 Hz), 6.89 (d, 1H, J=9.5 Hz), 4.32 (d, 2H, J=12.5 Hz), 4.02 (d, 2H, J=6.5 Hz), 2.84 (t, 2H, J=12 Hz), 2.65 (s, 3H), 2.07 (m, 1H), 1.86 (d, 2H, J=11.5 Hz), 1.34 (q, 2H, J=12.5 Hz). MS (M+1): 580.3

### Example 77

### (R)-3-((1-(5-(3-methyl-5-phenylbenzofuran-2-carboxamido)pyridin-2-yl)pyrrolidin-3-yl)methoxy)benzoic acid (77)

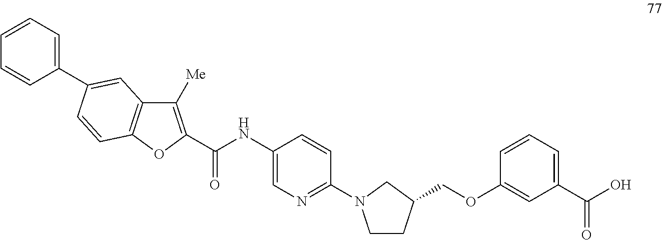

Compound 77 was prepared by the general procedure for compound 1 and subsequent hydrolysis. ¹H NMR (500 MHz, DMSO-d6) δ 10.29 (s, 1H), 8.46 (s, 1H), 8.04 (s, 1H), 7.96 (s, 1H), 7.93 (dd, 1H, J=8.5, 2.5 Hz), 7.80 (d, 1H, J=8.5 Hz), 7.76 (d, 2H, J=7.5 Hz), 7.73 (d, 1H, J=8.5 Hz), 7.54 (d, 1H, J=7.5 Hz), 7.50 (m, 3H), 7.42 (d, 1H, J=8 Hz), 7.39 (d, 1H, J=7.5 Hz), 7.22 (d, 1H, J=7 Hz), 6.50 (d, 1H, J=9 Hz), 4.11 (t, 1H, J=6.5 Hz), 4.06 (t, 1H, J=8 Hz), 3.65 (t, 1H, J=7.5 Hz), 3.55 (m, 1H), 3.42 (q, 1H, J=9.5 Hz), 3.31 (dd, 1H, J=10, 6.5 Hz), 2.80 (m, 1H), 2.64 (s, 3H), 2.19 (m, 1H), 1.91 (m, 1H). MS (M+1): 548.3

### Example 78

### 4-methyl-3-((1-(5-(3-methyl-5-phenylbenzofuran-2-carboxamido)pyridin-2-yl)piperidin-4-yloxy)methyl)benzoic acid (78)

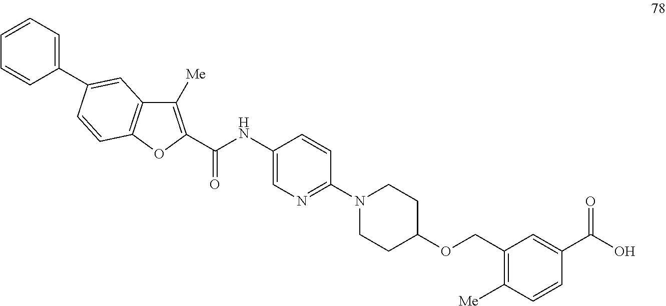

Compound 78 was prepared by the general procedure for compound 1 and subsequent hydrolysis. ¹H NMR (500 MHz, DMSO-d6) δ 10.72 (broad s, 1H), 8.63 (s, 1H), 8.26 (d, 1H, J=7.5 Hz), 8.07 (s, 1H), 7.83 (d, 1H, J=9 Hz), 7.76 (m, 3H), 7.51 (t, 2H, J=7.5 Hz), 7.47 (d, 1H, J=8 Hz), 7.43 (s, 1H), 7.40 (t, 1H, J=7 Hz), 7.38 (broad s, 1H), 7.27 (d, 1H, J=7.5 Hz), 4.33 (d, 2H, J=13 Hz), 3.95 (d, 2H, J=6 Hz), 3.18 (m, 2H), 2.66 (s, 3H), 2.23 (s, 3H), 2.18 (m, 1H), 1.96 (d, 2H, J=12.5 Hz), 1.46 (q, 2H, J=10 Hz). MS (M+1): 576.2

### Example 79

### 2-(3-((1-(5-(3-methyl-5-phenylbenzo furan-2-carboxamido)pyridin-2-yl)piperidin-4-yloxy)methyl)phenyl)acetic acid (79)

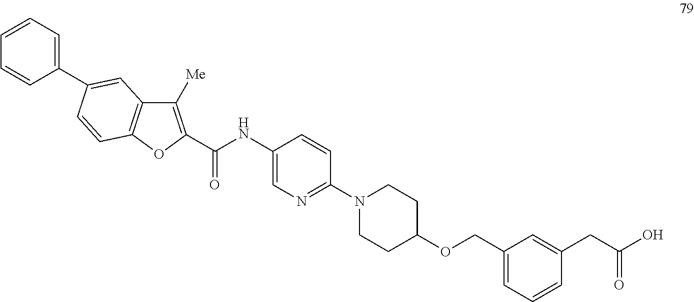

Compound 79 was prepared by the general procedure for compound 1 and subsequent hydrolysis. ¹H NMR (500 MHz, DMSO-d6) δ 10.36 (s, 1H), 8.51 (d, 1H, J=2 Hz), 8.04 (s, 1H), 7.95 (dd, 1H, J=9, 2.5 Hz), 7.80 (d, 1H, J=9 Hz), 7.76 (d, 2H, J=8 Hz), 7.73 (d, 1H, J=8.5 Hz), 7.50 (t, 2H, J=7.5 Hz), 7.39 (t, 1H, 7.5 Hz), 7.13 (t, 1H, J=8 Hz), 6.87 (d, 1H, J=9 Hz), 6.83 (s, 1H), 6.78 (d, 1H, J=7.5 Hz), 6.74 (d, 1H, J=8 Hz), 4.31 (d, 2H, 13 Hz), 3.82 (d, 2H, J=7 Hz), 3.33 (s, 2H), 2.82 (t, 2H, J=12 Hz), 2.65 (s, 3H), 2.00 (m, 1H), 1.85 (d, 2H, J=14 Hz), 1.31 (q, 2H, J=13.5 Hz). MS (M+1): 576.3

### Example 80

### (R)-4-((1-(5-(3-methyl-5-phenylbenzofuran-2-carboxamido)pyridin-2-yl)pyrrolidin-3-yl)methoxy)benzoic acid (80)

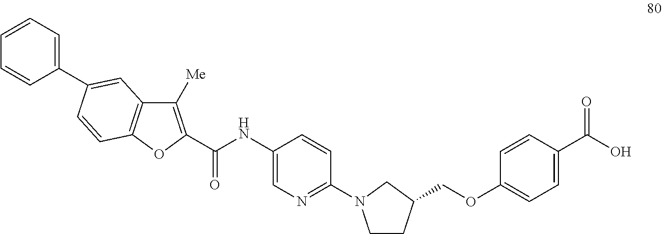

Compound 80 was prepared by the general procedure for compound 1 and subsequent hydrolysis. ¹H NMR (500 MHz, DMSO-d6) δ 12.67 (broad s, 1H), 10.63 (broad s, 1H), 8.55 (s, 1H), 8.20 (broad s, 1H), 8.07 (s, 1H), 7.91 (d, 2H, J=9 Hz), 7.82 (d, 1H, J=9 Hz), 7.75 (m, 3H), 7.51 (t, 2H, J=7.5 Hz), 7.40 (t, 1H, J=7.5 Hz), 7.06 (d, 2H, J=8.5 Hz), 6.99 (broad s, 1H), 4.18 (t, 1H, J=6.5 Hz), 4.11 (t, 1H, J=7.5 Hz), 3.76 (t, 1H, J=8.5 Hz), 3.66 (m, 1H), 3.55 (m, 1H), 3.41 (m, 1H), 2.89 (m, 1H), 2.66 (s, 3H), 2.24 (m, 1H), 1.97 (m, 1H). MS (M+1): 548.3

### Example 81

### (S)-4-((1-(5-(3-methyl-5-phenylbenzofuran-2-carboxamido)pyridin-2-yl)pyrrolidin-3-yl)methoxy)benzoic acid (81)

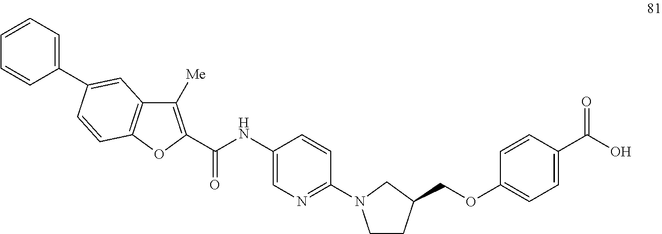

Compound 81 was prepared by the general procedure for compound 1 and subsequent hydrolysis. ¹H NMR (500 MHz, DMSO-d6) δ 10.78 (broad s, 1H), 8.63 (s, 1H), 8.31 (d, 1H, J=8.5 Hz), 8.08 (s, 1H), 7.91 (d, 2H, J=8 Hz), 7.83 (d, 1H, J=9 Hz), 7.75 (m, 3H), 7.51 (t, 2H, J=8 Hz), 7.40 (t, 1H, J=8 Hz), 7.17 (broad s, 1H), 7.06 (d, 2H, J=8 Hz), 4.18 (t, 1H, J=6 Hz), 4.11 (t, 1H, J=8.5 Hz), 3.82 (t, 1H, 8 Hz), 3.73 (m, 1H), 3.63 (m, 1H), 3.50 (m, 1H), 2.92 (m, 1H), 2.66 (s, 3H), 2.27 (m, 1H), 2.00 (m, Hi). MS (M+1): 548.3

### Example 82

### N-(2-fluorophenyl)-4-(5-(3-methyl-5-(oxazol-2-yl)benzofuran-2-carboxamido)pyridin-2-yl)piperazine-1-carboxamide (82)

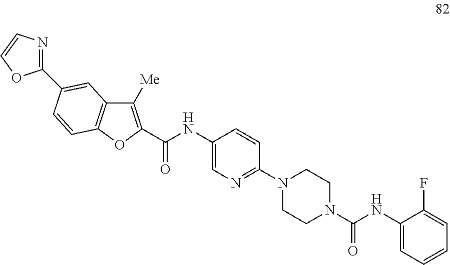

Compound 82 was prepared by the general procedure for compound 1. ¹H NMR (500 MHz, DMSO-d6) δ 10.46 (s, 1H), 8.55 (d, 1H, J=2.5 Hz), 8.42 (s, 1H), 8.37 (s, 1H), 8.28 (s, 1H), 8.15 (d, 1H, J=8.5 Hz), 8.01 (dd, 1H, J=9, 3 Hz), 7.82 (d, 1H, J=8.5 Hz), 7.46 (m, 1H), 7.43 (s, 1H), 7.20 (m, 1H), 7.13 (m, 2H), 6.94 (d, 1H, J=9.5 Hz), 3.57 (m, 4H), 3.54 (m, 4H), 2.66 (s, 3H). MS (M+1): 541.2

### Example 83

### methyl 3-((1-(5-(3-methyl-5-(oxazol-2-yl)benzofuran-2-carboxamido)pyridin-2-yl)piperidin-4-yl)methoxy)benzoate (83)

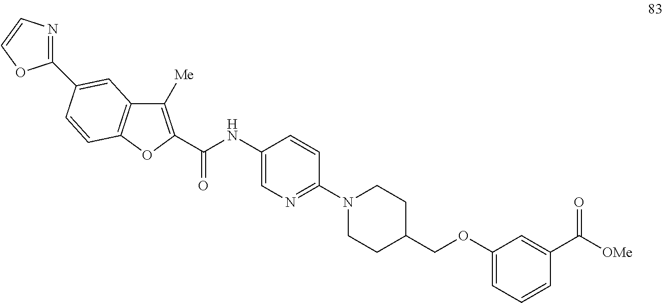

Compound 83 was prepared by the general procedure for compound 1. ¹H NMR (500 MHz, CDCl₃) δ 8.37 (d, 1H, J=1.5 Hz), 8.32 (d, 1H, J=2.5 Hz), 8.21 (m, 2H), 8.08 (dd, 1H, J=9, 3 Hz), 7.78 (s, 1H), 7.65 (d, 1H, J=8 Hz), 7.61 (d, 1H, J=8.5 Hz), 7.58 (broad s, 1H), 7.37 (t, 1H, J=8 Hz), 7.13 (dd, 1H, J=8, 2.5 Hz), 6.76 (d, 1H, J=9 Hz), 4.37 (d, 2H, J=13 Hz), 3.94 (s, 3H), 3.92 (d, 211, J=6.5 Hz), 2.94 (t, 2H, J=13 Hz), 2.74 (s, 3H), 2.11 (m, 1H), 1.99 (d, 2H, J=12.5 Hz), 1.48 (qd, 2H, J=12, 3.5 Hz). MS (M+1): 567.2

### Example 84

### 3-((1-(5-(3-methyl-5-(oxazol-2-yl)benzofuran-2-carboxamido)pyridin-2-yl)piperidin-4-yl)methoxy)benzoic acid (84)

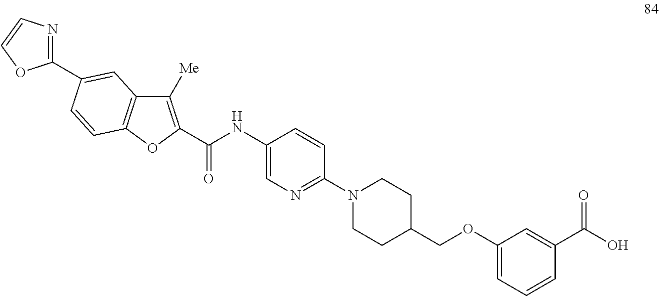

Compound 84 was prepared by the general procedure for compound 1 and subsequent hydrolysis. ¹H NMR (500 MHz, DMSO-d6) δ 10.78 (broad s, 1H), 8.61 (s, 1H), 8.38 (s, 1H), 8.28 (s, 1H), 8.26 (m, 1H), 8.17 (d, 1H, J=8.5 Hz), 7.83 (d, 1H, J=9 Hz), 7.53 (d, 1H, J=8 Hz), 7.46 (s, 1H), 7.43 (m, 3H), 7.21 (d, 1H, J=8 Hz), 4.31 (d, 2H, J=13 Hz), 3.95 (d, 2H, J=6 Hz), 3.19 (m, 2H), 2.67 (s, 3H), 2.16 (m, 1H), 1.94 (d, 2H, J=13 Hz), 1.42 (q, 2H, J=10.5 Hz). MS (M+1): 553.2

### Example 85

### methyl 3-((1-(5-(3,6,7-trimethylbenzofuran-2-carboxamido)pyridin-2-yl)piperidin-4-yloxy)methyl)benzoate (85)

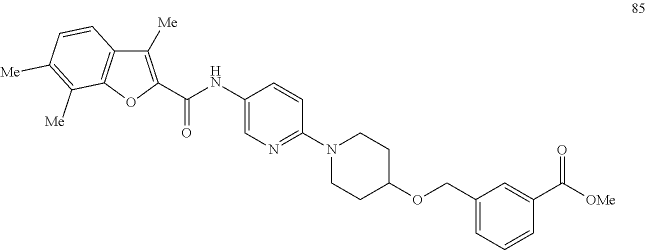

Compound 85 was prepared by the general procedure for compound 1. ¹H NMR (500 MHz, CDCl₃) δ 8.31 (d, 1H, J=2.5 Hz), 8.15 (s, 1H), 8.06 (m, 2H), 7.99 (d, 1H, J=8 Hz), 7.61 (d, 1H, J=7.5 Hz), 7.46 (t, 1H, =7.5 Hz), 7.37 (d, 1H, J=8 Hz), 7.15 (d, 1H, J=8 Hz), 6.75 (d, 1H, J=9 Hz), 4.66 (s, 2H), 4.02 (m, 2H), 3.95 (s, 3H), 3.69 (m, 1H), 3.25 (td, 2H, J=9.5, 3.5 Hz), 2.66 (s, 3H), 2.51 (s, 3H), 2.49 (s, 3H), 2.04 (m, 2H), 1.77 (m, 2H). MS (M+1): 528.2

### Example 86

### 3-((1-(5-(3-methyl-5-(oxazol-2-yl)benzofuran-2-carboxamido)pyridin-2-yl)piperidin-4-yloxy)methyl)benzoic acid (86)

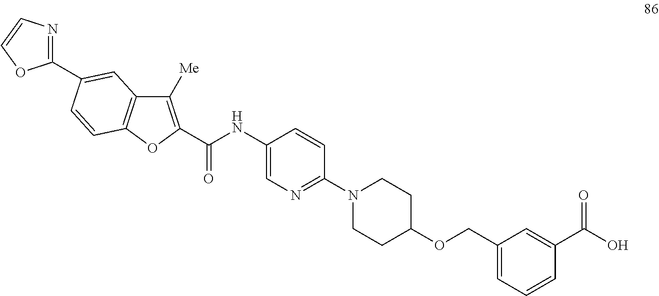

Compound 86 was prepared by the general procedure for compound 1 and subsequent hydrolysis. ¹H NMR (500 MHz, DMSO-d6) δ 10.41 (s, 1H), 8.50 (d, 1H, J=2.5 Hz), 8.37 (s, 1H), 8.27 (s, 1H), 8.15 (d, 1H, J=9 Hz), 7.95 (d, 1H, J=9 Hz), 7.94 (s, 1H), 7.86 (d, 1H, f=7.5 Hz), 7.82 (d, 1H, J=9 Hz), 7.60 (d, 1H, J=7.5 Hz), 7.49 (t, 1H, J=7.5 Hz), 7.42 (s, 1H), 6.90 (d, 1H, J=9.5 Hz), 4.63 (s, 2H), 3.96 (m, 2H), 3.66 (m, 1H), 3.16 (t, 2H, J=10 Hz), 2.65 (s, 3H), 1.97 (m, 2H), 1.53 (q, 2H, J=9 Hz). MS (M+1): 553.2

### Example 87

### 3-((1-(5-(3,6,7-trimethylbenzofuran-2-carboxamido)pyridin-2-yl)piperidin-4-yloxy)methyl)benzoic acid (87)

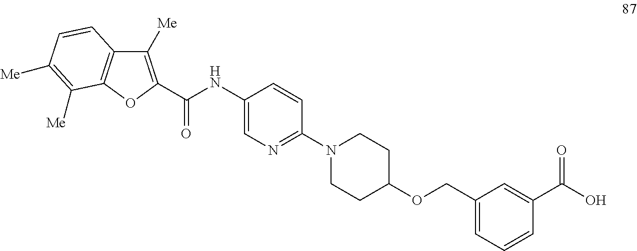

Compound 87 was prepared by the general procedure for compound 1 and subsequent hydrolysis. NMR (500 MHz, DMSO-d6) δ 10.08 (s, 1H), 8.45 (d, 1H, J=2 Hz), 7.94 (s, 1H), 7.87 (m, 2H), 7.60 (d, 1H, J=7 Hz), 7.48 (t, 1H, J=8 Hz), 7.45 (d, 1H, J=8 Hz), 7.17 (d, 1H, J=8 Hz), 6.91 (d, 1H, J=9.5 Hz), 4.64 (s, 2H), 3.97 (m, 2H), 3.67 (m, 1H), 3.16 (t, 2H, J=10 Hz), 2.54 (s, 3H), 2.51 (s, 3H), 2.39 (s, 3H), 1.97 (m, 2H), 1.54 (q, 2H, J=9 Hz). MS (M+1): 514.2

### Example 88

### methyl 3-((1-(5-(3-methyl-5-(thiazol-2-yl)benzofuran-2-carboxamido)pyridin-2-yl)piperidin-4-yl)methoxy)benzoate (88)

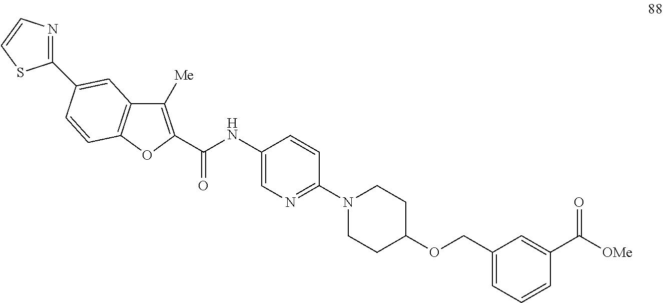

Compound 88 was prepared by the general procedure for compound 1. ¹H NMR (500 MHz, CDCl₃) δ 8.32 (d, 1H, J=2.5 Hz), 8.29 (s, 1H), 8.21 (s, 1H), 8.09 (m, 2H), 7.92 (d, 1H, J=3.5 Hz), 7.66 (d, 1H, J=7.5 Hz), 7.60 (d, 1H, J=8.5 Hz), 7.58 (m, 1H), 7.39 (d, 1H, J=3 Hz), 7.36 (d, 1H, J=7.5 Hz), 7.13 (dd, 1H, J=8, 3 Hz), 6.76 (d, 1H, J=9 Hz), 4.37 (d, 2H, J=13 Hz), 3.94 (s, 3H), 3.92 (d, 2H, J=6 Hz), 2.93 (1, 2H, J=12 Hz), 2.75 (s, 3H), 2.11 (m, 1H), 1.99 (d, 2H, J=12.5 Hz), 1.48 (qd, 211, J=12.5, 4 Hz). MS (M+1): 583.2

### Example 89

### methyl 3-((1-(5-(3-methyl benzo furan-2-carboxamido)pyridin-2-yl)piperidin-4-yl)methoxy)benzoate (89)

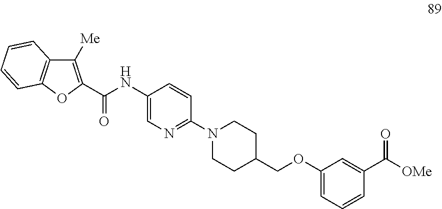

Compound 89 was prepared by the general procedure for compound 1. ¹H NMR (500 MHz, CDCl₃) δ 8.31 (d, 1H, J=2.5 Hz), 8.22 (s, 1H), 8.10 (dd, 1H, J=9, 3 Hz), 7.66 (t, 211, J=8.5 Hz), 7.58 (m, 1H), 7.54 (d, 1H, J=8 Hz), 7.48 (t, 1H, J=7.5 Hz), 7.37 (d, 1H, J=8 Hz), 7.34 (d, 1H, J=7 Hz), 7.13 (dd, 1H, J=7.5, 2.5 Hz), 6.76 (d, 1H, J=9.5 Hz), 4.36 (d, 2H, J=12.5 Hz), 3.94 (s, 3H), 3.91 (d, 2H, J=6.5 Hz), 2.93 (td, 2H, J=12.5, 2 Hz), 2.70 (s, 3H), 2.11 (m, 1H), 1.99 (d, 2H, J=12.5 Hz), 1.48 (qd, 2H, J=12.5, 4.5 Hz). MS (M+1): 500.2

### Example 90

### 3-((1-(5-(3-methyl-5-(thiazol-2-yl)benzofuran-2-carboxamido)pyridin-2-yl)piperidin-4-yl)methoxy)benzoic acid (90)

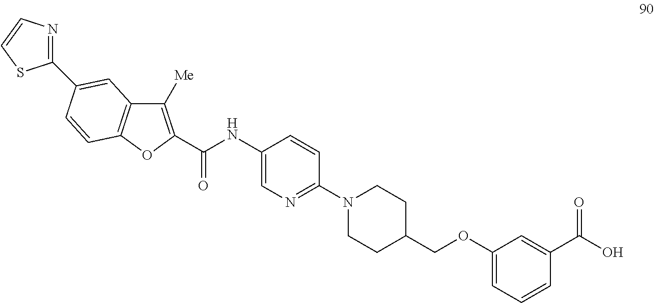

Compound 90 was prepared by the general procedure for compound 1 and subsequent hydrolysis. ¹H NMR (500 MHz, DMSO-d6) δ 10.39 (s, 1H), 8.50 (d, 1H, J=2 Hz), 8.34 (s, 1H), 8.12 (d, 1H, J=9 Hz), 7.96 (m, 2H), 7.82 (d, 1H, J=3.5 Hz), 7.78 (d, 1H, J=9 Hz), 7.52 (d, 1H, J=8 Hz), 7.45 (s, 1H), 7.40 (t, 1H, J=7.5 Hz), 7.20 (d, 1H, J=7.5 Hz), 6.88 (d, 1H, J=9 Hz), 4.32 (d, 2H, J=12.5 Hz), 3.92 (d, 2H, 6 Hz), 2.83 (t, 2H, J=11.5 Hz), 2.65 (s, 3H), 2.04 (m, 1H), 1.86 (d, 2H, J=11.5 Hz), 1.33 (q, 2H, J=12.5 Hz). MS (M+1): 569.2

### Example 91

### 3-((1-(5-(3-methylbenzofuran-2-carboxamido)pyridin-2-yl)piperidin-4-yl)methoxy)benzoic acid (91)

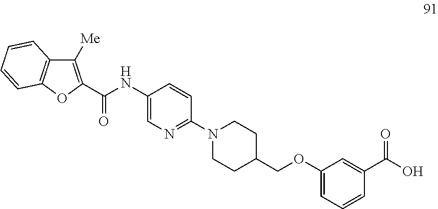

Compound 91 was prepared by the general procedure for compound 1 and subsequent hydrolysis. ¹H NMR (500 MHz, DMSO-d6) δ 10.74 (s, 1H), 8.63 (s, 1H), 8.28 (d, 1H, J=7.5 Hz), 7.82 (d, 1H, J=8 Hz), 7.68 (d, 1H, J=8 Hz), 7.54 (m, 2H), 7.42 (m, 4H), 7.21 (d, 1H, J=8 Hz), 4.32 (d, 2H, J=13 Hz), 3.95 (d, 2H, J=6.5 Hz), 3.21 (t, 2H, J=11 Hz), 2.61 (s, 3H), 2.16 (m, 1H), 1.94 (d, 2H, J=13 Hz), 1.43 (q, 2H, J=12 Hz). MS (M+1): 486.2

### Example 92

### methyl 3-((1-(5-(5-benzyl-3-methylbenzofuran-2-carboxamido)pyridin-2-yl)piperidin-4-yl)methoxy)benzoate (92)

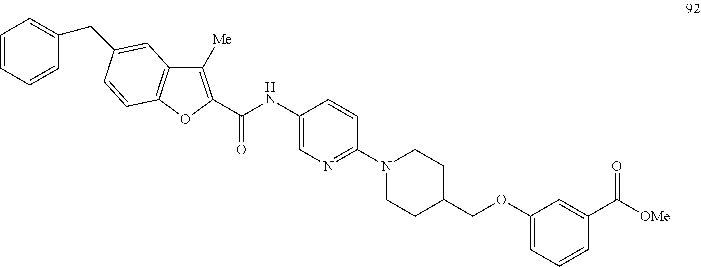

Compound 92 was prepared by the general procedure for compound 1. ¹H NMR (500 MHz, CDCl₃) δ 8.30 (d, 1H, J=2.5 Hz), 8.19 (s, 1H), 8.09 (dd, 1H, J=9, 3 Hz), 7.65 (d, 1H, J=7.5 Hz), 7.58 (s, 1H), 7.47 (s, 1H), 7.44 (d, 1H, J=8.5 Hz), 7.33 (m, 4H), 7.25 (m, 3H), 7.13 (dd, 1H, J=8.5, 2.5 Hz), 6.75 (d, 1H, J=9 Hz), 4.36 (d, 2H, J=13 Hz), 4.14 (s, 2H), 3.94 (s, 3H), 3.91 (d, 2H, J=6 Hz), 2.93 (t, 2H, J=13 Hz), 2.66 (s, 3H), 2.11 (m, 1H), 1.99 (d, 2H, J=13.5 Hz), 1.48 (qd, 2H, J=12.5, 4 Hz). MS (M+1): 590.3

### Example 93

### 3-((1-(5-(5-benzyl-3-methylbenzofuran-2-carboxamido)pyridin-2-yl)piperidin-4-yl)methoxy)benzoic acid (93)

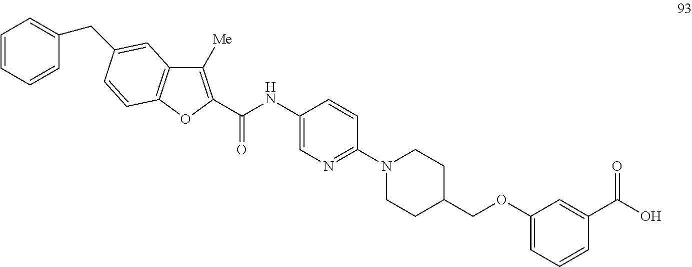

Compound 93 was prepared by the general procedure for compound 1 and subsequent hydrolysis. ¹H NMR (500 MHz, DMSO-d6) δ 10.56 (broad s, 1H), 8.56 (s, 1H), 8.16 (broad s, 1H), 7.67 (s, 1H), 7.58 (d, 1H, J=8.5 Hz), 7.53 (d, 1H, J=7.5 Hz), 7.45 (s, 1H), 7.42 (d, 1H, J=7.5 Hz), 7.39 (d, 1H, J=8.5 Hz), 7.29 (m, 4H), 7.20 (m, 2H), 4.28 (d, 2H, J=13 Hz), 4.09 (s, 2H), 3.94 (d, 2H, J=6 Hz), 3.07 (m, 2H), 2.57 (s, 3H), 2.12 (m, 1H), 1.92 (d, 2H, J=13 Hz), 1.39 (q, 2H, J=10.5 Hz). MS (M+1): 576.3

### Example 94

### 3-((1-(5-(3-methyl-5-phenoxybenzofuran-2-carboxamido)pyridin-2-yl)piperidin-4-yl)methoxy)benzoic acid (94)

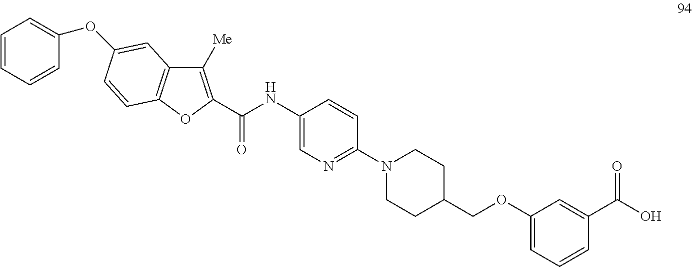

Compound 94 was prepared by the general procedure for compound 1 and subsequent hydrolysis. ¹H NMR (500 MHz, DMSO-d6) δ 13.02 (broad s, 1H), 10.34 (s, 1H), 8.50 (s, 1H), 7.94 (d, 1H, J=9 Hz), 7.69 (d, 1H, J=8 Hz), 7.52 (d, 1H, J=8 Hz), 7.45 (s, 2H), 7.40 (m, 3H), 7.22 (m, 2H), 7.13 (t, 1H, J=6.5 Hz), 7.01 (d, 2H, J=8.5 Hz), 6.88 (d, 1H, J=9.5 Hz), 4.32 (d, 2H, J=13.5 Hz), 3.92 (d, 2H, J=6.5 Hz), 2.83 (t, 2H, J=12.5 Hz), 2.51 (s, 3H), 2.04 (m, 1H), 1.86 (d, 2H, J=11 Hz), 1.33 (q, 2H, J=12 Hz). MS (M+1): 578.2

### Example 95

### 4-(5-(3-methyl-5-phenylbenzofuran-2-carboxamido)-2,3′-bipyridin-6′-yloxy)cyclohexanecarboxylic acid (95)

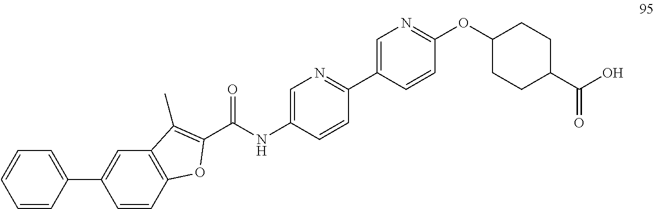

Compound 95 was prepared by the general procedure for compound 1 and subsequent hydrolysis. (2:1 trans:cis ratio). ¹H NMR (500 MHz, DMSO-d6) δ 12.15 (bs, 1H), 10.81 (s, 1H), 9.10 (m, 1H), 8.85 (m, 1H), 8.35 (m, 2H), 8.09 (s, 1H), 7.98 (d, 1H, J=9.0 Hz), 7.77-7.78 (m, 4H), 7.52 (t, 2H, J=7.5 Hz), 7.40 (t, 1H, J=7.5 Hz), 6.87 (d, 1H, J=9.0 Hz), 74.99-5.04 (m, 1H), 2.69 (s, 3H), 2.26-2.31 (m, 1H), 1.98-2.15 (m, 4H), 1.43-1.56 (m, 4H). MS (M+1): 548.3.

### Assay

A useful assay to determine the DGAT inhibitory activity of the inventive compounds is described below:

The in vitro assay to identify DGAT1 inhibitors uses human DGAT1 enzyme expressed in Sf9 insect cells prepared as microsomes. The reaction was initiated by the addition of the combined substrates 1,2-dioleoyl-sn-glycerol and [¹⁴C]-palmitoyl-Co A and incubated with test compounds and microsomal membranes for 2 hours at room temperature. The assay was stopped by adding 0.5 mg wheat germ agglutinin beads in assay buffer with 1% Brij-35 and 1% 3-cholamidopropyldimethyl-ammonio-1-propane sulfonate. Plates were sealed with TopSeal and incubated for 18 hours to allow the radioactive triglyceride product to come into proximity with the bead. Plates were read on a TopCount instrument.

Percent inhibition was calculated as the percent of (test compound inhibition minus non-specific binding) relative to (total binding minus non-specific binding). IC₅₀ values were determined by curve fitting the data to a Sigmoidal dose-response in GraphPad Prism utilizing the following equation:

Y=A+(B−A)/(1+10̂((Log IC₅₀−X))),

where A and B are the bottom and top of the curve (highest and lowest inhibition), respectively, and X is the logarithm of concentration.

| Example | IC50 (nM) |
|---|---|
| 1 | 25 |
| 2 | 625 |
| 3 | 589 |
| 4 | 46 |
| 5 | 39 |
| 6 | 24 |
| 7 | 42 |
| 8 | 48 |
| 9 | 49 |
| 10 | 37 |
| 11 | 95 |
| 12 | 103 |
| 13 | 282 |
| 14 | 361 |
| 17 | 125 |
| 18 | 52 |
| 19 | 70 |
| 20 | 115 |
| 21 | 566 |
| 24 | 59 |
| 25 | 93 |
| 26 | 59 |
| 27 | 316 |
| 29 | 134 |
| 30 | 1420 |
| 31 | 84 |
| 33 | 93 |
| 35 | 38 |
| 36 | 90 |
| 37 | 106 |
| 38 | 1009 |
| 39 | 55 |
| 40 | 100 |
| 41 | 145 |
| 42 | 55 |
| 43 | 51 |
| 44 | 60 |
| 45 | 40 |
| 46 | 84 |
| 50 | 2333 |
| 51 | 28 |
| 52 | 33 |
| 53 | 27 |
| 54 | 220 |
| 55 | 78 |
| 57 | 240 |
| 58 | 13 |
| 59 | 9 |
| 62 | 482 |
| 63 | 774 |
| 64 | 65 |
| 65 | 454 |
| 66 | 454 |
| 68 | 2458 |
| 69 | 204 |
| 70 | 1087 |
| 71 | 171 |
| 72 | 13265 |
| 73 | 108 |
| 74 | 120 |
| 75 | 311 |
| 76 | 65 |
| 77 | 152 |
| 78 | 156 |
| 80 | 600 |
| 81 | 1056 |
| 82 | 104 |
| 84 | 344 |
| 86 | 305 |
| 87 | 22 |
| 90 | 194 |
| 91 | 84 |
| 93 | 56 |
| 94 | 71 |
| 95 | 6 |

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

1. A compound, or pharmaceutically acceptable salt of said compound, the compound being represented by the formula I: 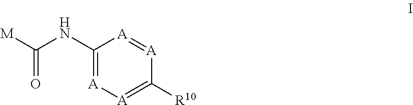
wherein:
each A is independently selected from C(R⁴) or N;
M is a bicyclic moiety selected from:
either 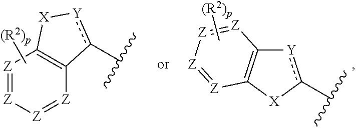
wherein each Z is independently selected from C(R³) or N;
each X is independently selected from C, O, N, or S;
each Y is independently selected from C(R³) or N;
p is 1-4; [symbol]
refers to an optional bond; and
R² is independently selected from H, alkyl-, haloalkyl-, halo, alkoxy-, (alkoxy)alkyl-, haloalkoxy-, cycloalkyl-, (cycloalkyl)alkyl-, aryl-, (aryl)alkyl-, heteroaryl-, (heteroaryl)alkyl-, heterocyclyl-, and (heterocyclyl)alkyl-, wherein each of said alkyl, haloalkyl, alkoxy, (alkoxy)alkyl, heteroaryl-, (heteroaryl)alkyl-, heterocyclyl-, and (heterocyclyl)alkyl can be independently unsubstituted or optionally independently substituted with one or more moieties which are the same or different, each substituent being independently selected from the group consisting of alkyl, alkoxy, alkoxyalkyl, haloalkoxy, alkenyl, alkynyl, cycloalkyl, cycloalkylalkyl, cycloalkenyl, cycloalkenylalkyl, aryl, arylalkyl, heteroaryl, heteroarylalkyl, halo, —CN, ═OR^{c}, ═O, —C(O)R^{c},
—C(O)OR^{c}, —C(O)N(R^{c})(R^{d}), —SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}),
—N(R^{c})(R^{d}), -alkyl-N(R^{c})(R^{d}), —N(R^{c})C(O)R^{d}, —CH₂—N(R^{c})C(O)R^{d},
—CH₂—N(R^{c})C(O)N(R^{d})(R^{b}), —CH₂—R^{c}; —CH₂N(R^{c})(R^{d}), —N(R^{c})S(O)R^{d},
—N(R^{c})S(O)₂R^{d}, —CH₂—N(R^{c})S(O)₂R^{d}, —N(R^{c})S(O)₂N(R^{d})(R^{b}),
—N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{c}, —N₃, —NO₂ and
—S(O)₂R^{c}, wherein each R^{b}, R^{c} and R^{d} is independently selected;
R¹⁰ is either (i) a 4-8 membered heterocyclyl ring having from 1 to 3 ring N atoms, or (ii) a bicyclic heterocyclyl ring having from 1 to 3 ring N atoms, or (iii) an aryl group, or (iv) a heteroaryl group,
wherein each of said aryl or heteroaryl group for R¹⁰ independently is unsubstituted or optionally independently substituted with one or more G moieties wherein said G moieties can be the same or different, each G moiety being independently selected from the group shown below,
and further wherein each of said heterocyclyl ring and bicyclic heterocyclyl ring for R¹⁰ independently is unsubstituted or optionally substituted, off of either (i) a ring N atom or (ii) a ring carbon atom on said heterocyclyl ring or said bicyclic heterocyclyl ring, with one or more G moieties wherein said G moieties can be the same or different, each G moiety being independently selected from the group consisting of:
(a) 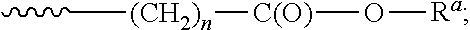
(b) 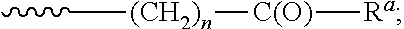
(c) 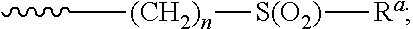
(d) 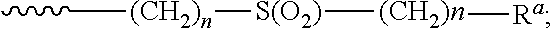
(e) 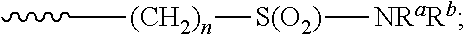
(f) 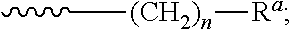
(g) 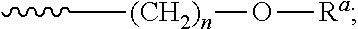
(h) 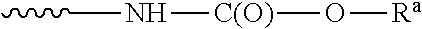
(i) 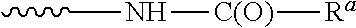
(J) 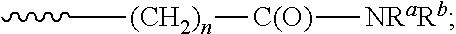
(k) 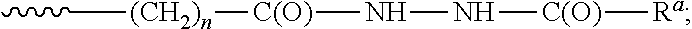
(l) 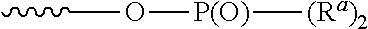
(m) 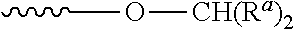
(n) an oxo group off of only C and not off of N; and
(o) 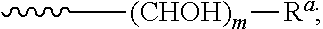
wherein R^{a} is selected from the group consisting of
hydrogen, hydroxy, CN, halo, alkyl, alkenyl, alkynyl, aryl, heteroaryl, heterocyclyl, cycloalkyl or spirocyclyl, wherein each of said alkyl, alkenyl, alkynyl, aryl, heteroaryl, heterocyclyl and cycloalkyl is unsubstituted or optionally independently substituted with one or more moieties which are the same or different, each moiety being selected independently from the group consisting of O-haloalkyl, S-haloalkyl, CN, NO₂, CF₃, cycloalkyl, heterocyclyl, haloalkyl, aryl, heteroaryl,
N-alkyl, N-haloalkyl, and N-cycloalkyl; alkyl, alkenyl, alkynyl, cycloalkylalkyl, cycloalkenyl, heterocyclylalkyl, aryl, arylalkyl, heteroaryl, heteroarylalkyl, halo, —OR^{c}, —C(O)R^{c}, —C(O)Oalkyl,
—C(O)Ocycloalkyl, —COOH, -alkylCOOH, —COOH bioisostere,
-alkylCOOH bioisostere, —C(O)N(R^{c})(R^{d}), —SF₅, —OSF₅, —Si(R^{c})₃, —SR^{c},
—S(O)N(R^{c})(R^{d}), —CH(R^{c})(R^{d}), —S(O)₂N(R^{c})(R^{d}), —C(═NOR^{c})R^{d},
—P(O)(OR^{c})(OR^{d}), —N(R^{c})(R^{d}), -alkyl-N(R^{c})(R^{d}), —N(R^{c})C(O)R^{d},
—CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d},
—N(R^{c})S(O)₂N(R^{d})(R^{b}), —N(R^{c})S(O)N(R^{d})(R^{b}), —N(R^{c})C(O)N(R^{d})(R^{b}),
—CH₂—N(R^{c})C(O)N(R^{d})(R^{b}), —N(R^{c})C(O)OR^{d}, —CH₂—N(R^{c})C(O)OR^{d},
—S(O)R^{c}, ═NOR^{c}, —N₃, and —S(O)₂R^{c};
wherein each R^{b}, R^{c} and R^{d} is independently selected;
R^{b} is H, lower alkyl, cycloalkyl, aryl, heteroaryl or heterocycloalkyl;
R^{c} is H, lower alkyl, cycloalkyl, aryl, heteroaryl or heterocycloalkyl;
R^{d} is H, lower alkyl, cycloalkyl, aryl, heteroaryl or heterocycloalkyl;
wherein each of said alkyl, cycloalkyl, aryl, heteroaryl or heterocycloalkyl in R^{b}, R^{c}, and R^{d} can be unsubstituted or optionally independently substituted with 1-2 substituents independently selected from halo, OH, NH₂, CF₃, CN, Oalkyl, NHalkyl, N(alkyl)₂ and Si(alkyl)₃;
R³ is independently selected from H, alkyl, haloalkyl, cycloalkyl, and (cycloalkyl)alkyl-;
R⁴ is independently selected from H, alkyl, haloalkyl, cycloalkyl, and (cycloalkyl)alkyl-;
m is 1-3, and
n is 0-3.

2. A compound or pharmaceutically acceptable salt of claim 1, wherein the moiety: 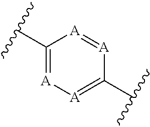
is

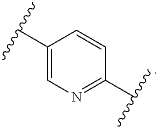

3. A compound or pharmaceutically acceptable salt thereof of claim 1 wherein, one Z is N and the other Z moieties are C(R³).

4. A compound or pharmaceutically acceptable salt thereof of claim 1 wherein, X is O.

5. A compound or pharmaceutically acceptable salt thereof of claim 1 wherein, X is S.

6. A compound or pharmaceutically acceptable salt thereof of any one of claims 4-5 wherein, Y is C(R³).

7. A compound or pharmaceutically acceptable salt thereof of claim 6 wherein, R² is selected from the group consisting of alkyl, halo and aryl.

8. A compound or pharmaceutically acceptable salt thereof of claim 7 wherein, R³ is hydrogen.

9. A compound or pharmaceutically acceptable salt thereof of claim 1 wherein, R¹⁰ is a 4-8 membered heterocyclyl ring having from 1 to 3 ring N atoms, wherein said heterocyclyl ring is substituted off of a ring N atom.

10. A compound or pharmaceutically acceptable salt thereof of claim 9 wherein, R¹⁰ is piperidine or the moiety:

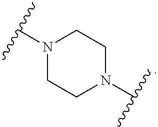

11. A compound or pharmaceutically acceptable salt thereof of claim 10 wherein, G is selected from the group consisting of:
(a) 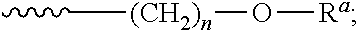
(b) 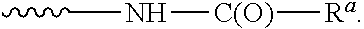
off of only C and not off of N; and
(c) 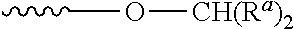
off of only C and not off of N.

12. A compound or pharmaceutically acceptable salt thereof of claim 11 wherein, R^{a} is aryl substituted as described in claim 1.

13. A compound, or a pharmaceutically acceptable salt of said compound, wherein the compound is selected from the group consisting of the following:

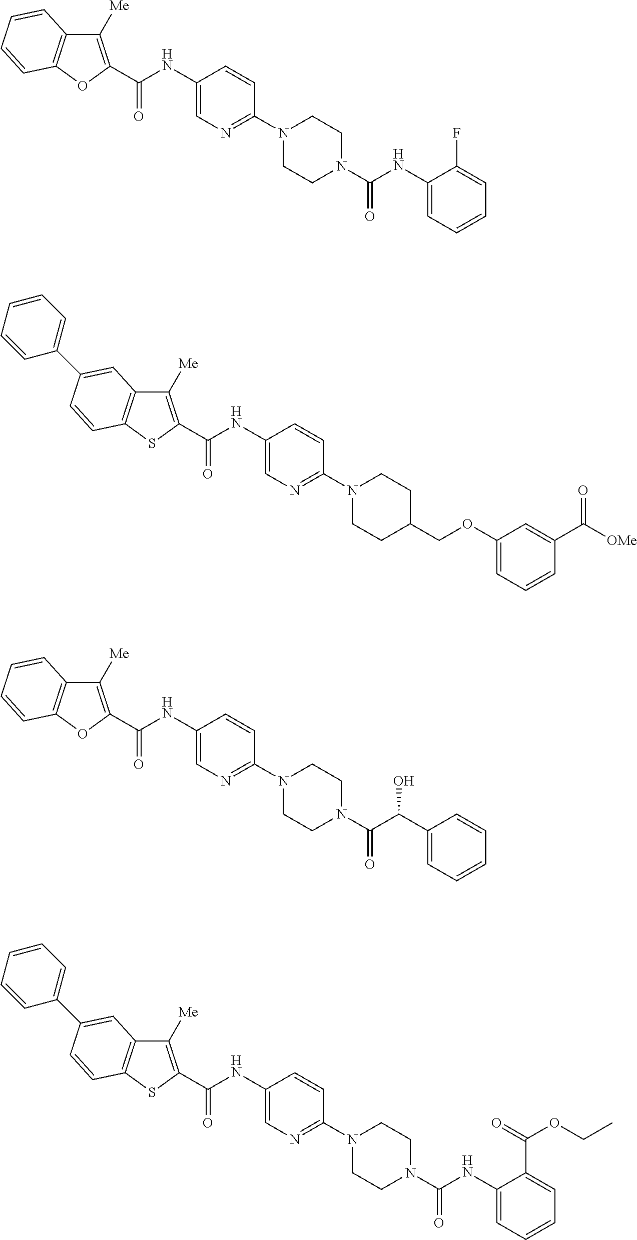

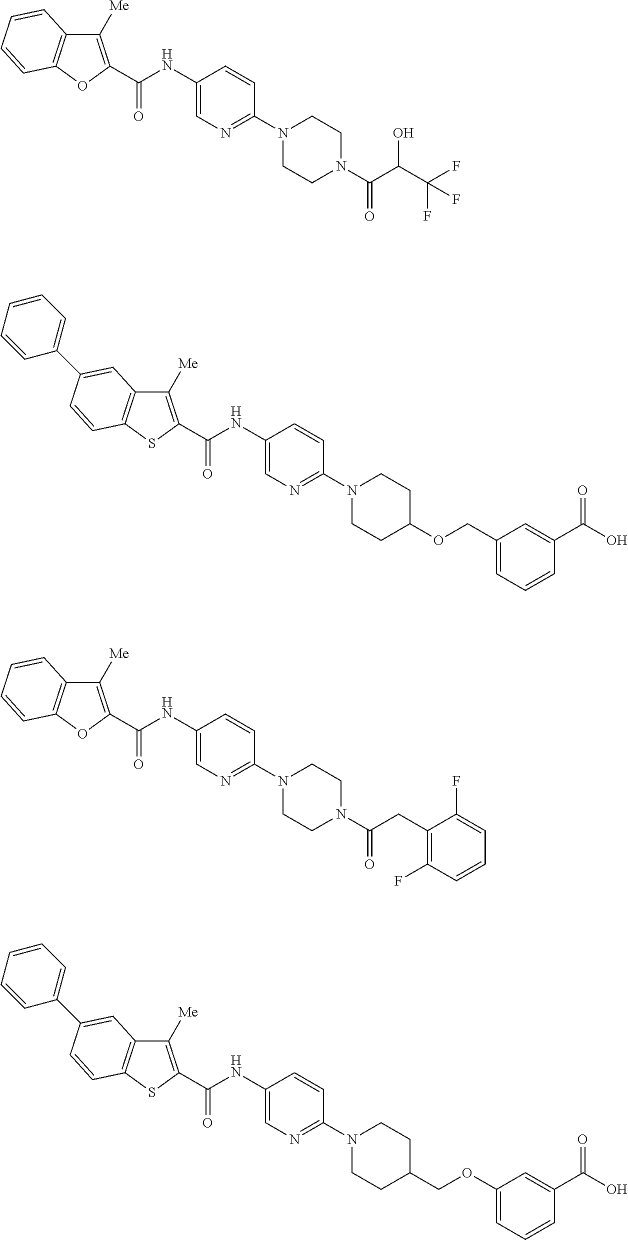

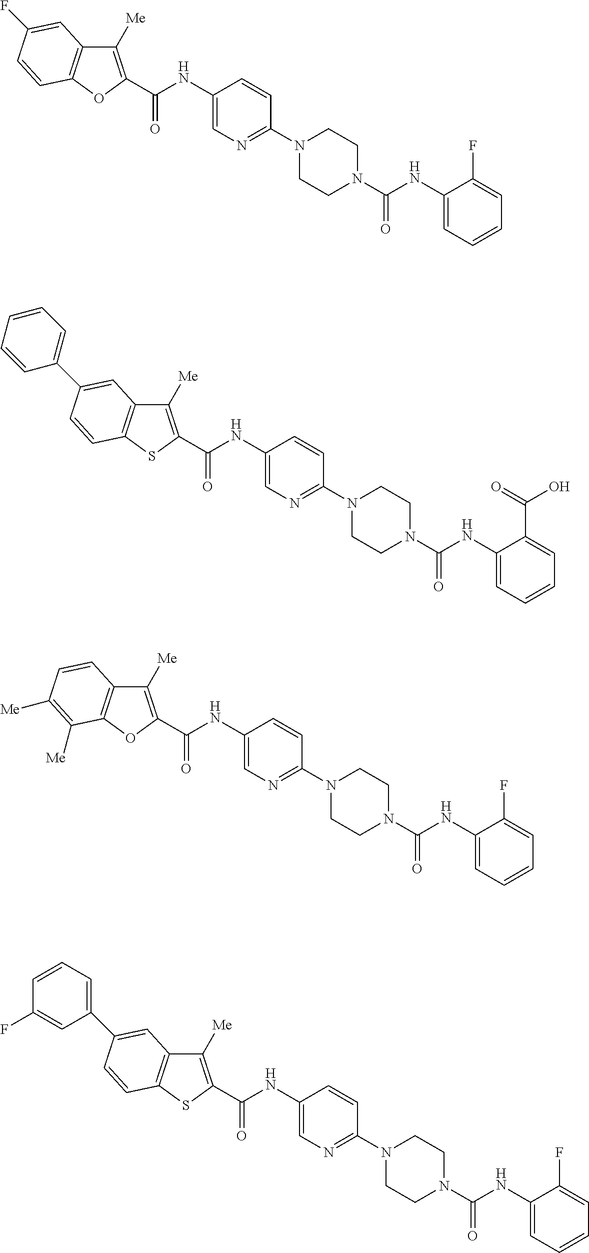

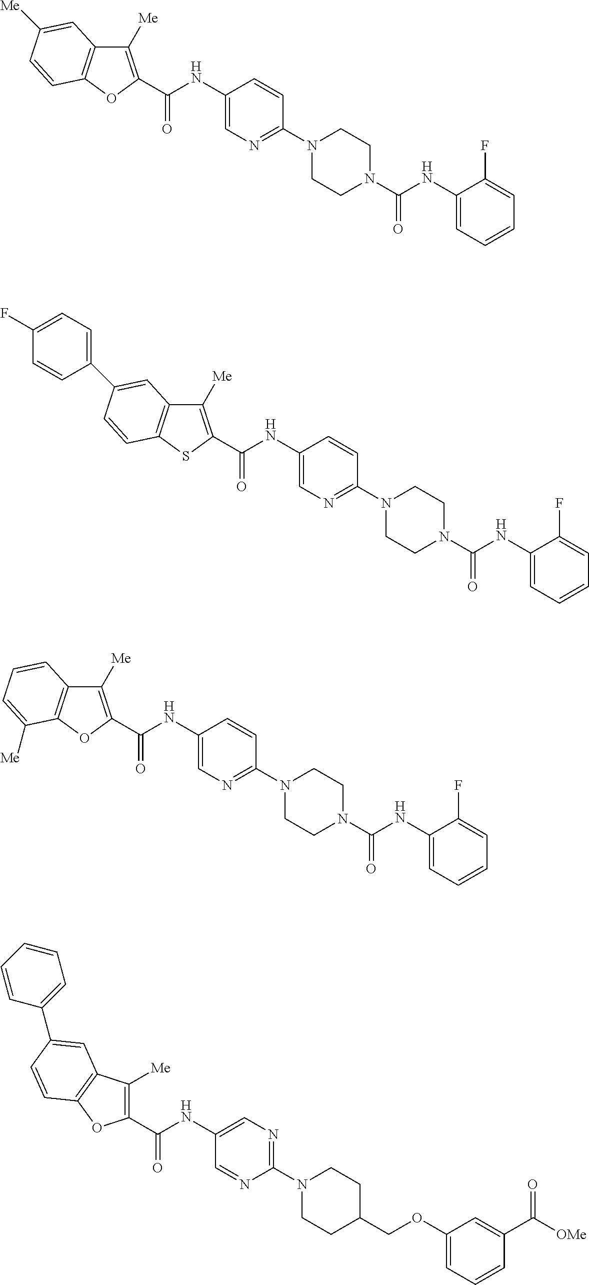

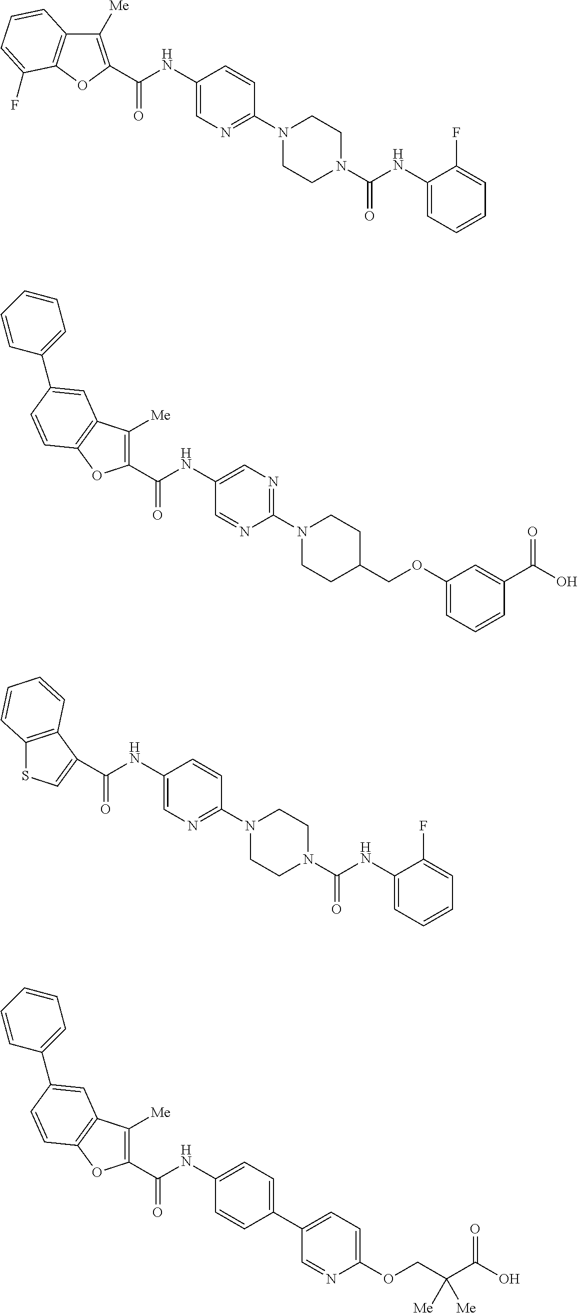

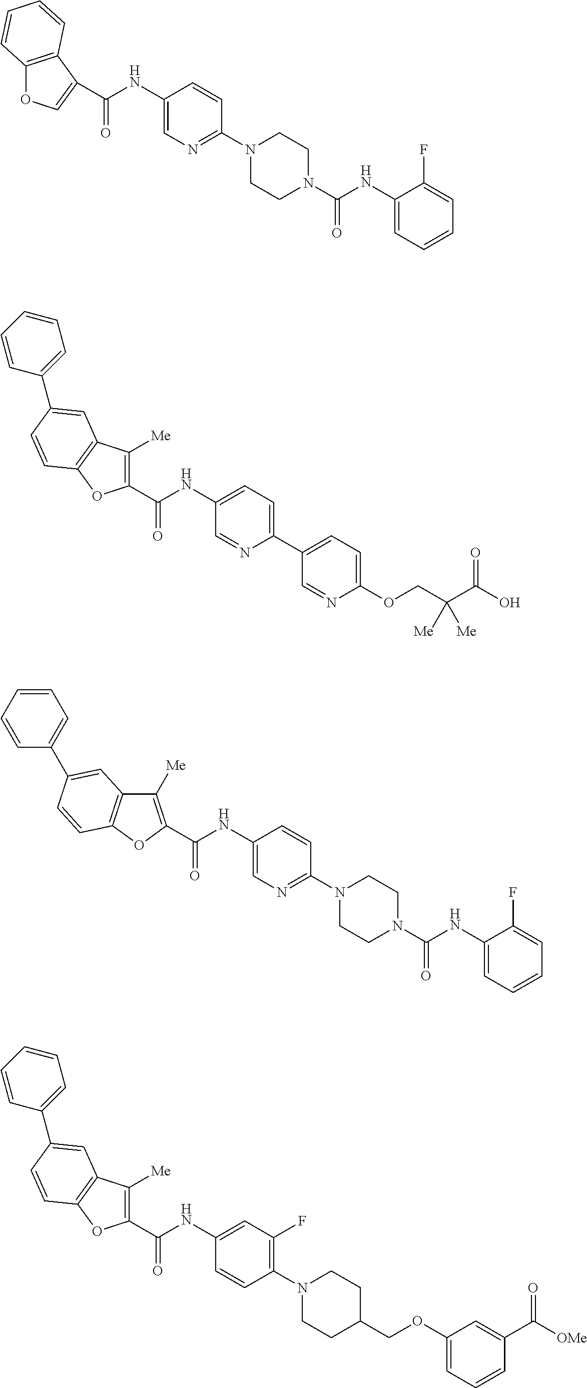

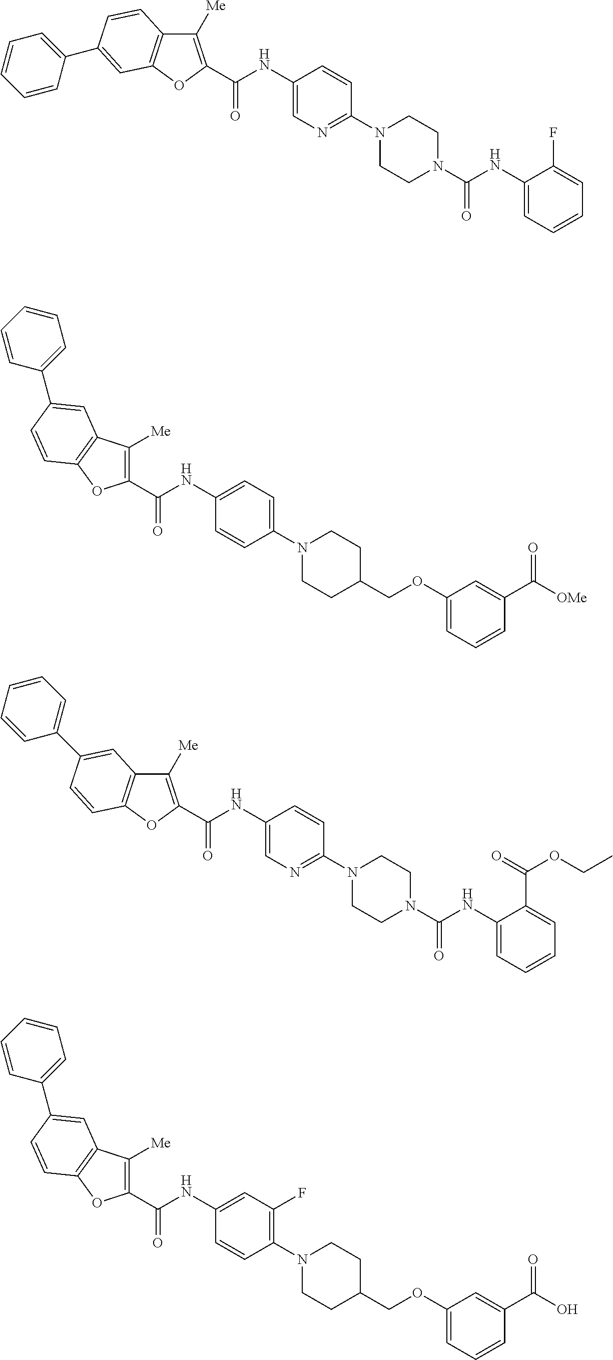

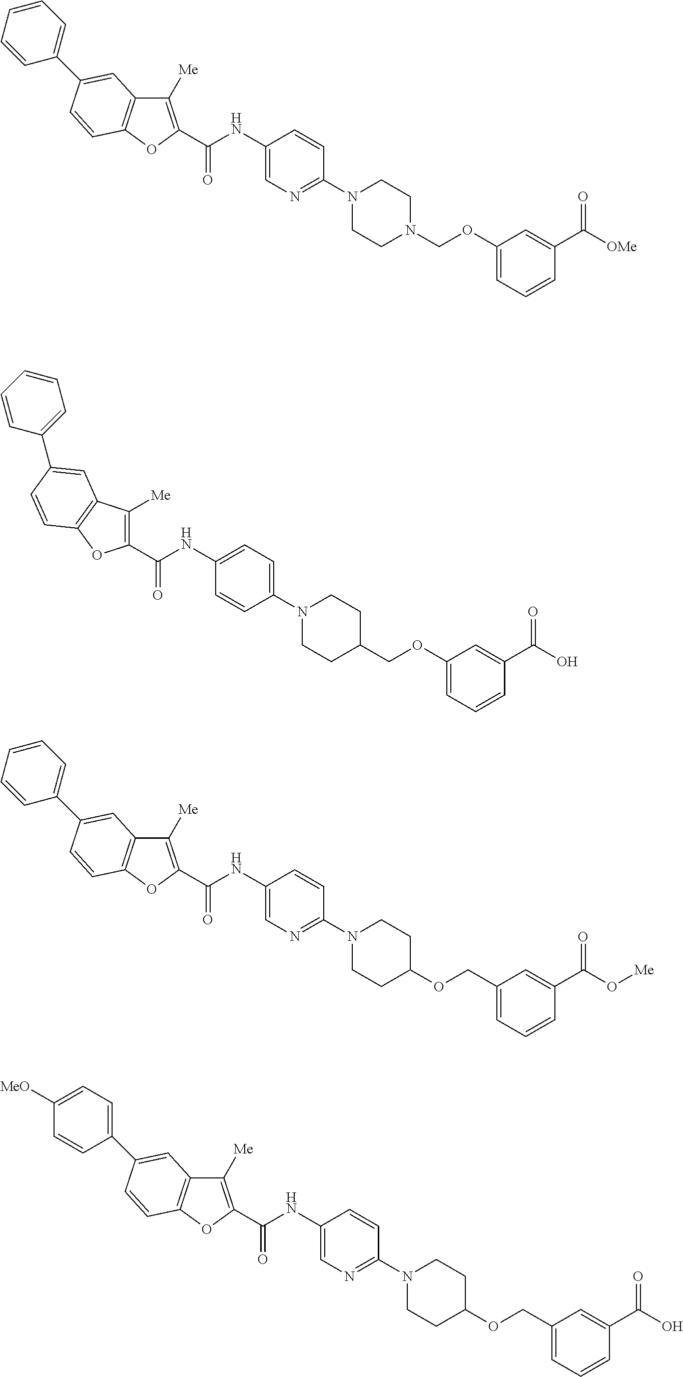

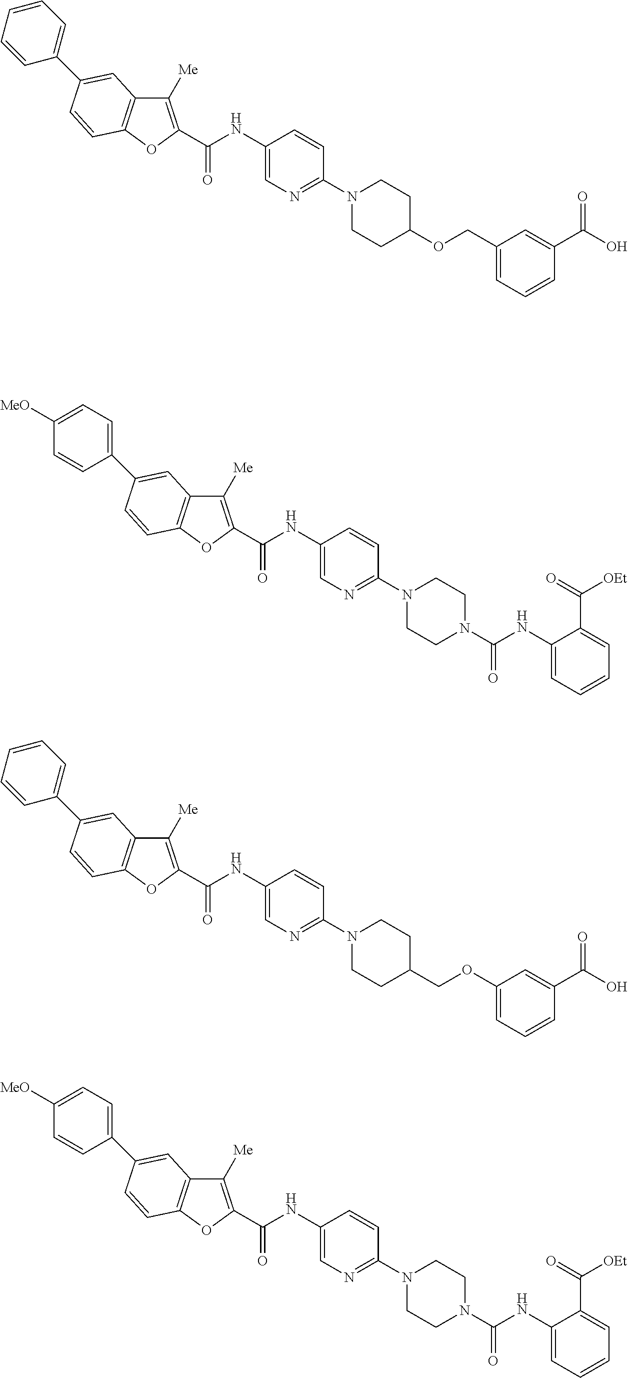

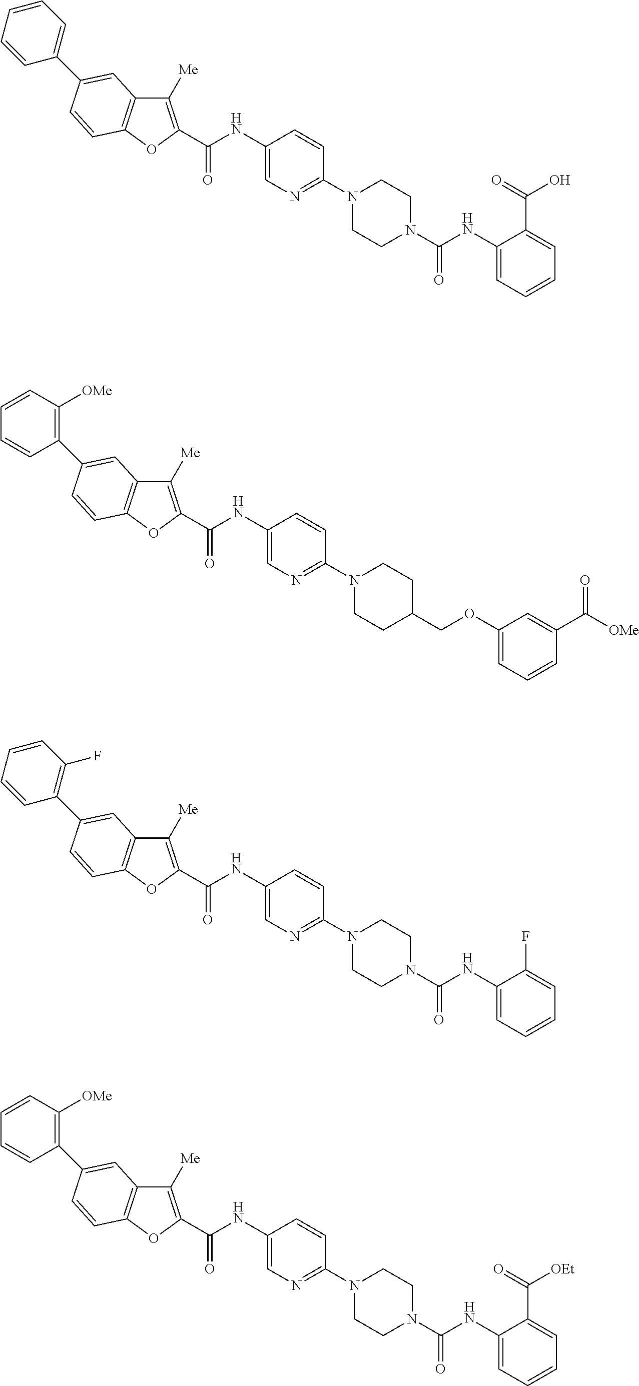

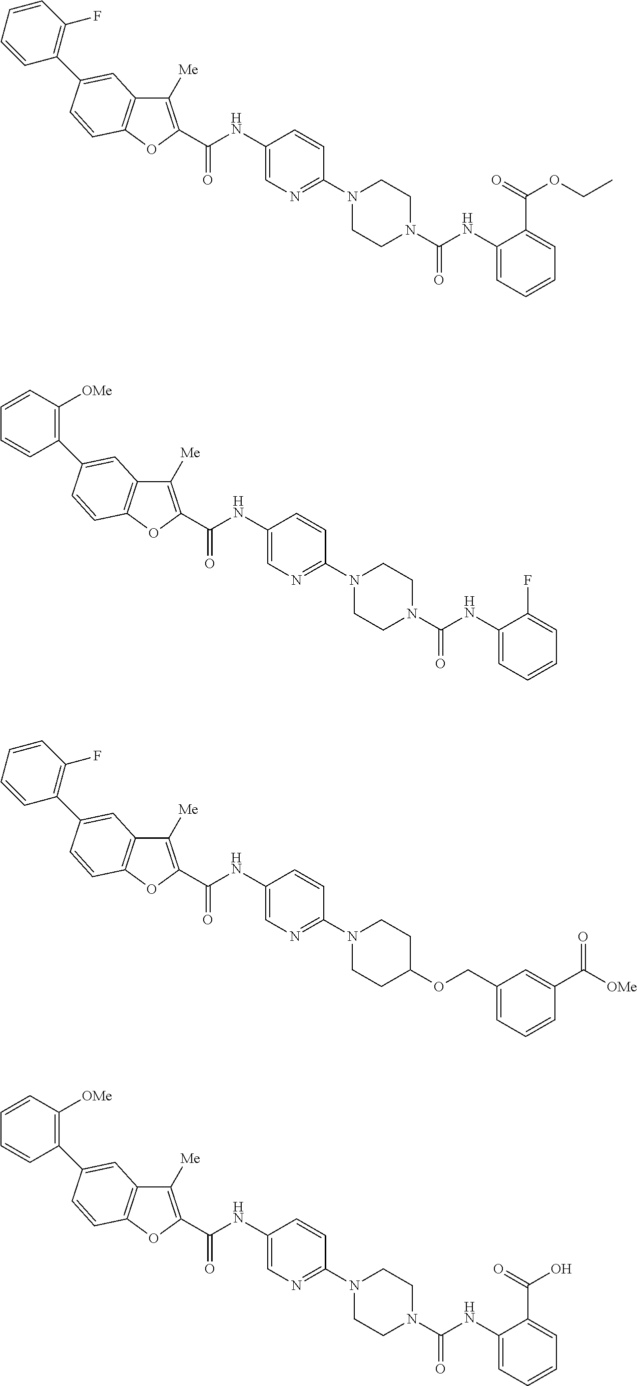

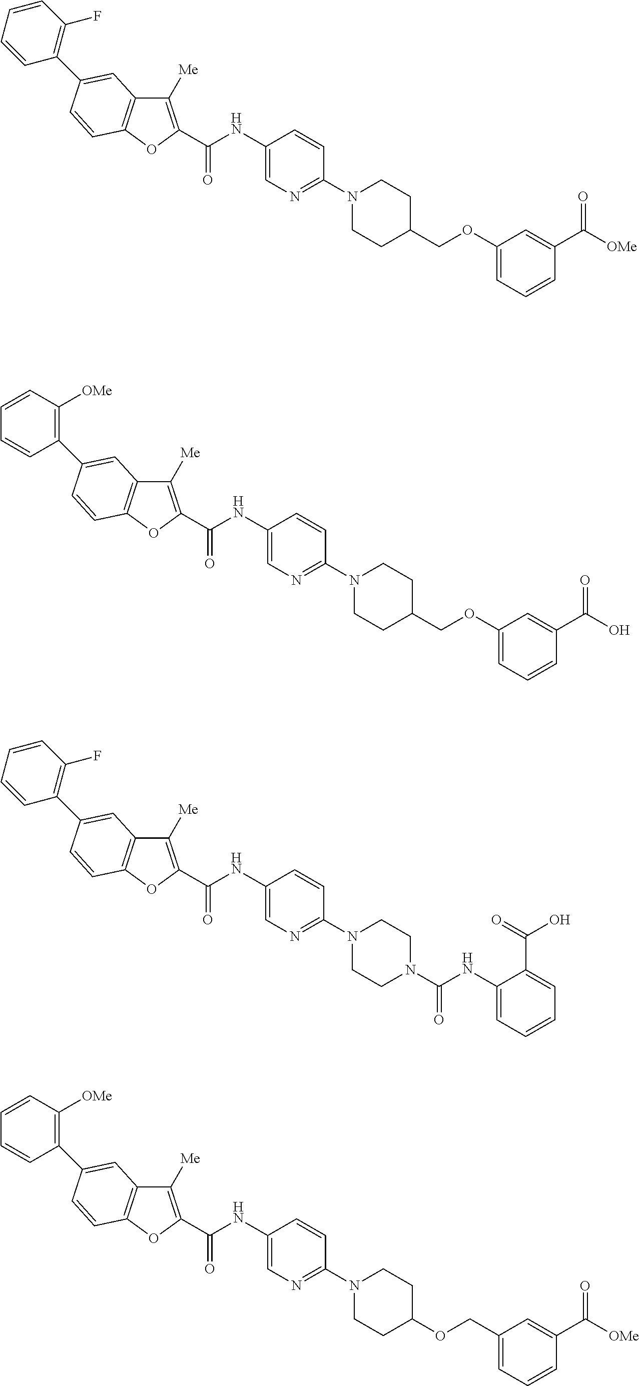

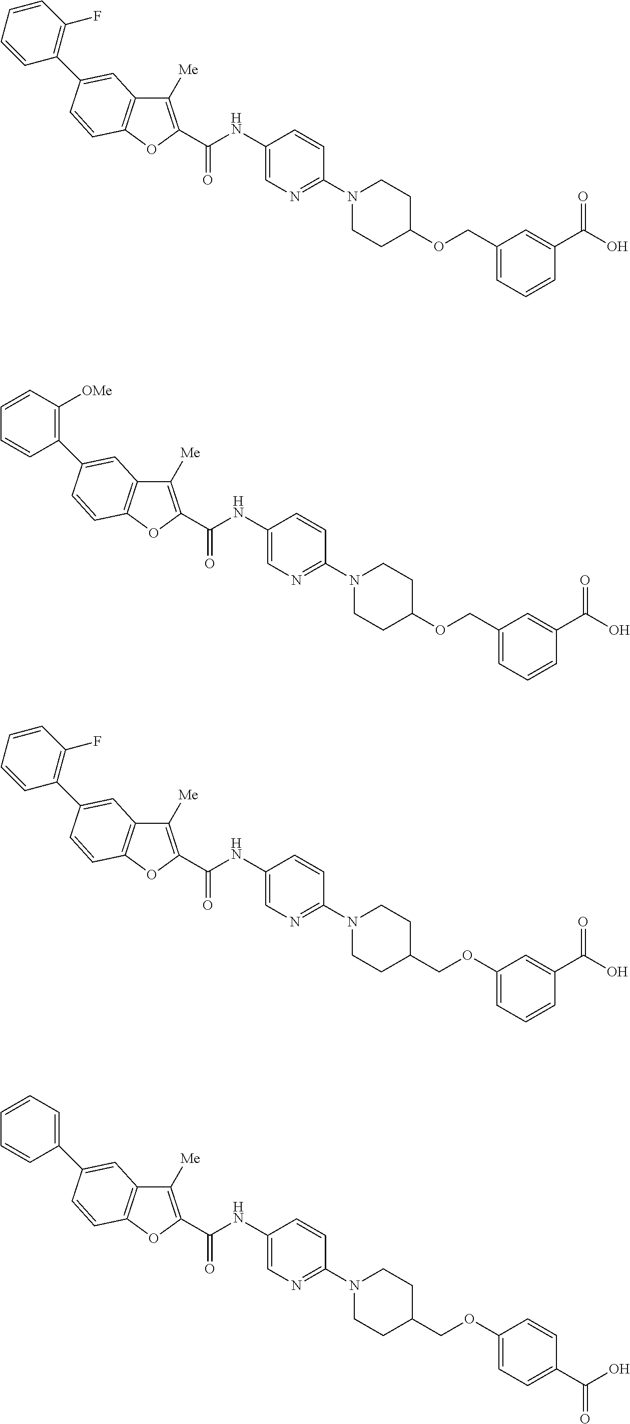

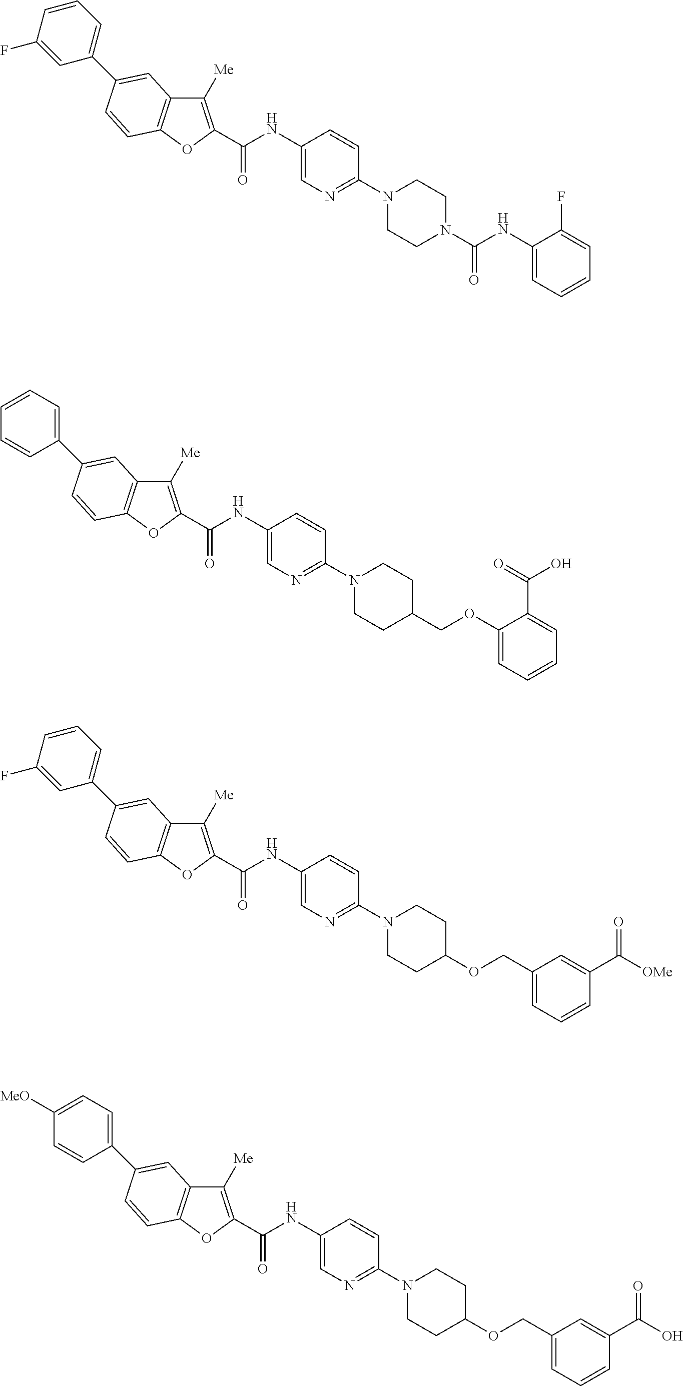

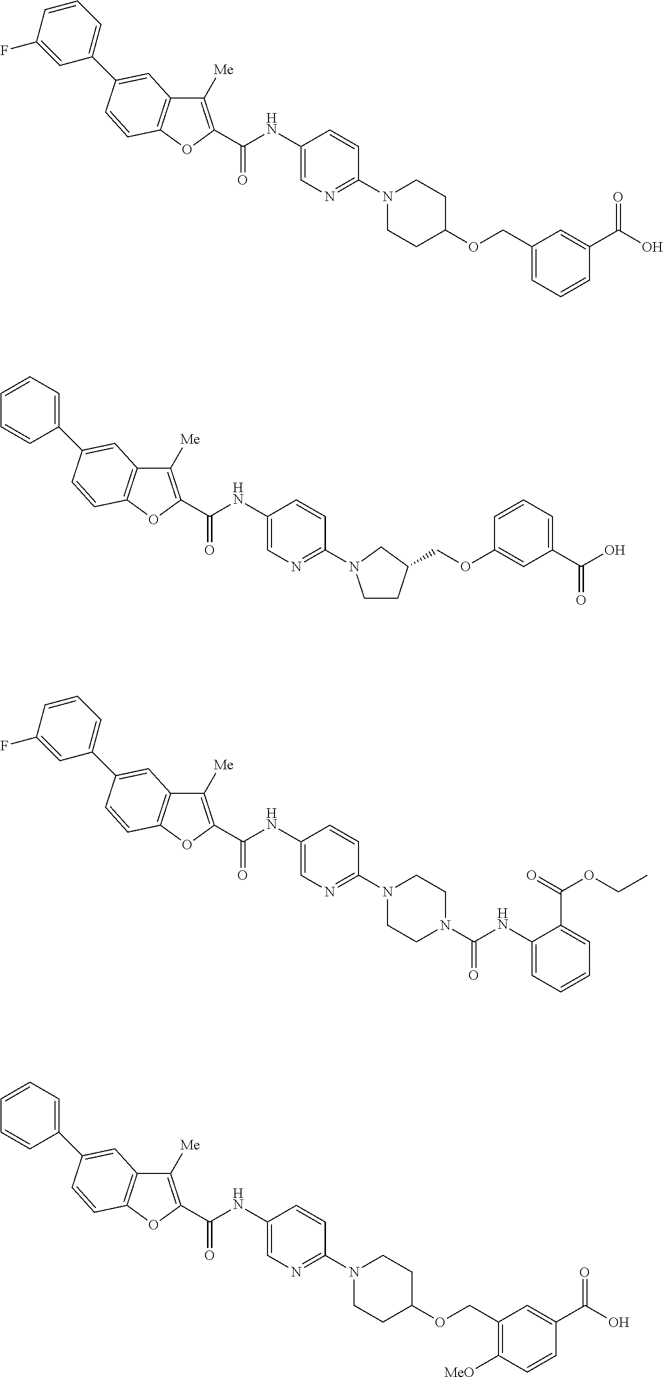

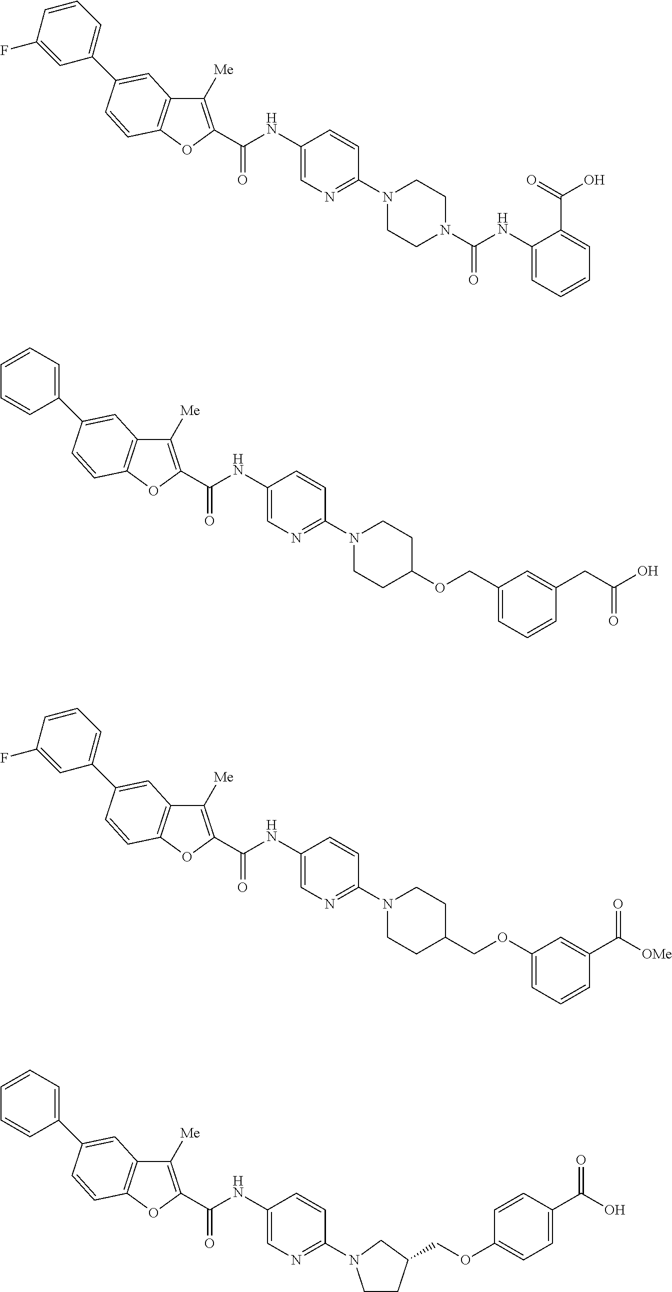

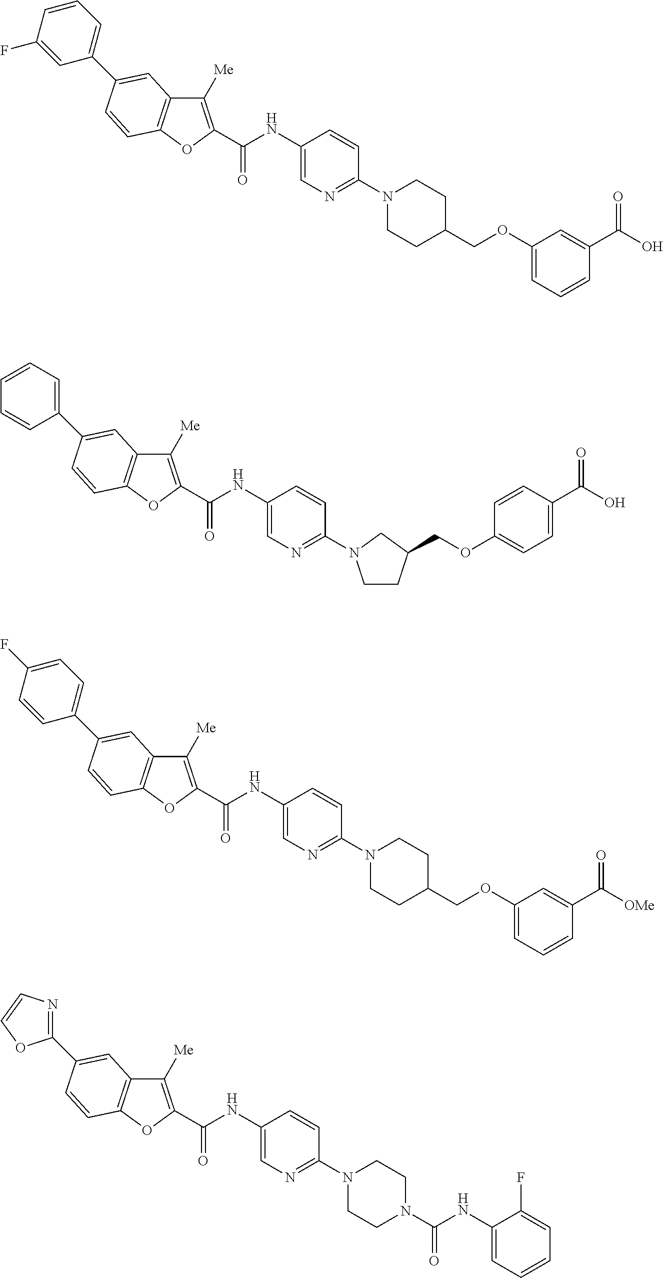

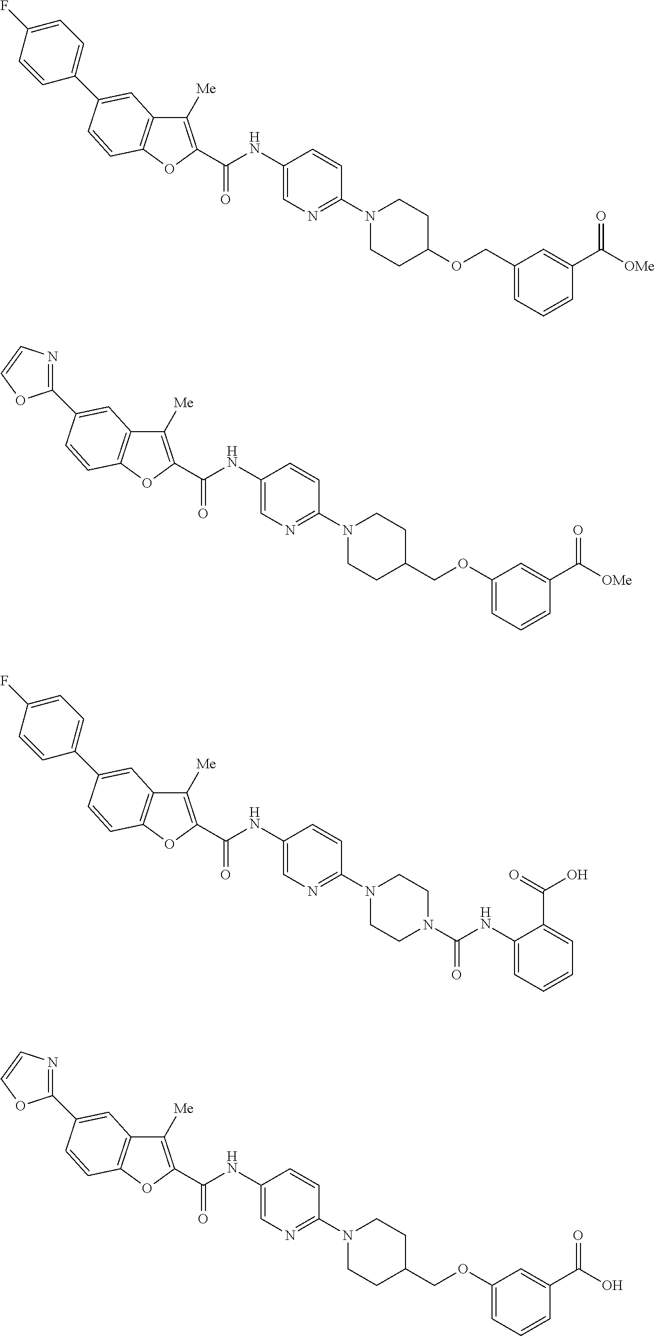

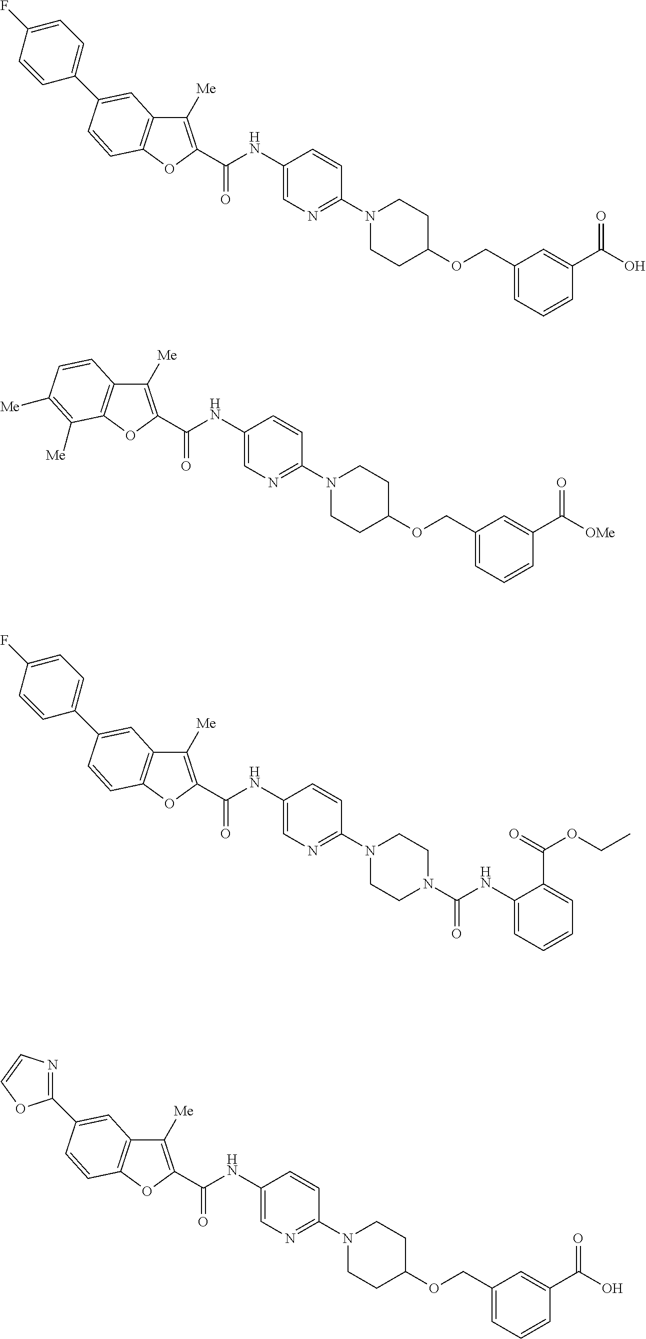

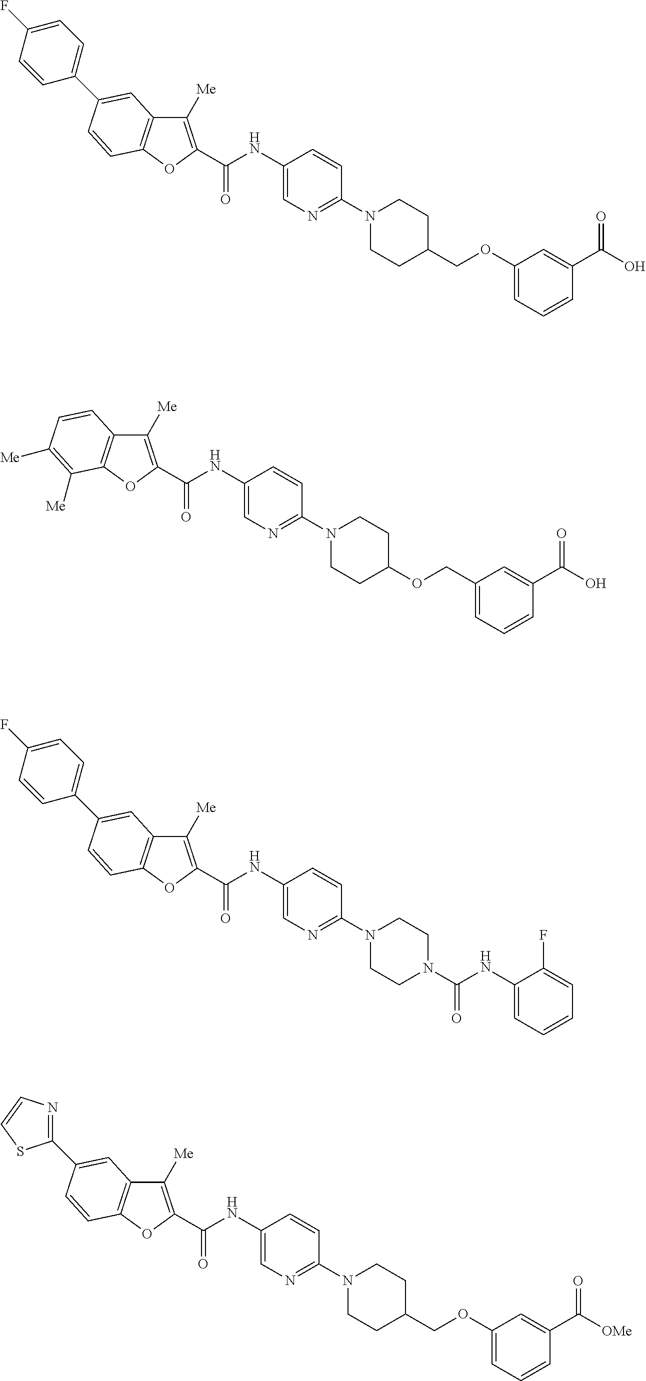

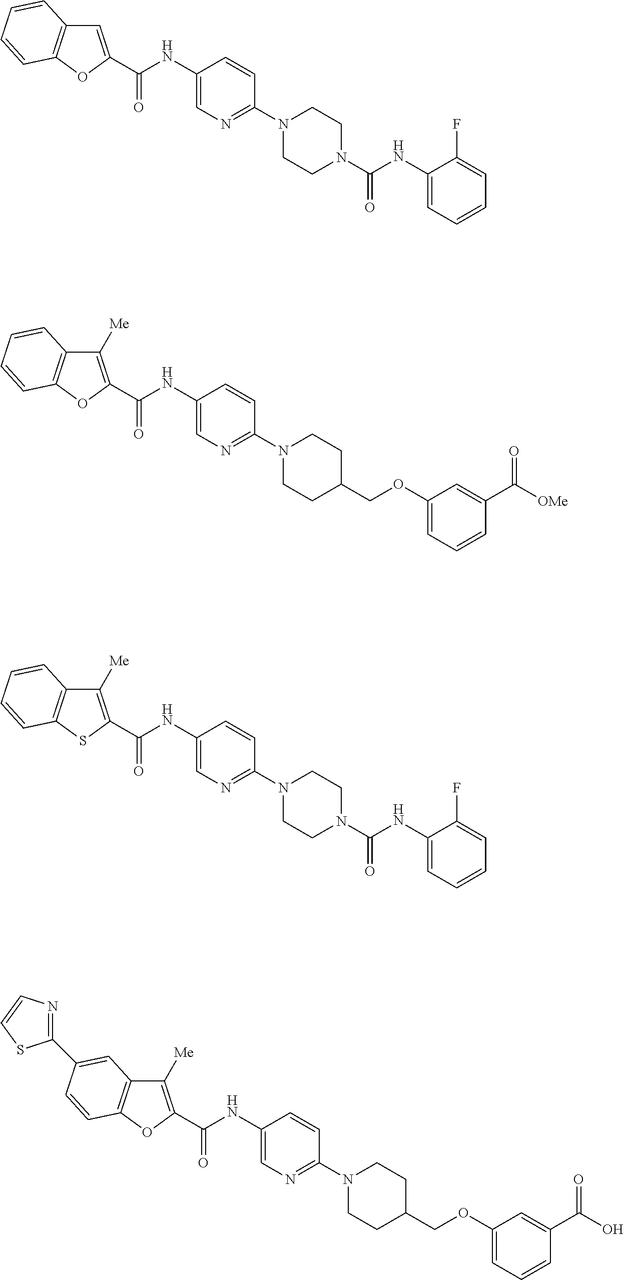

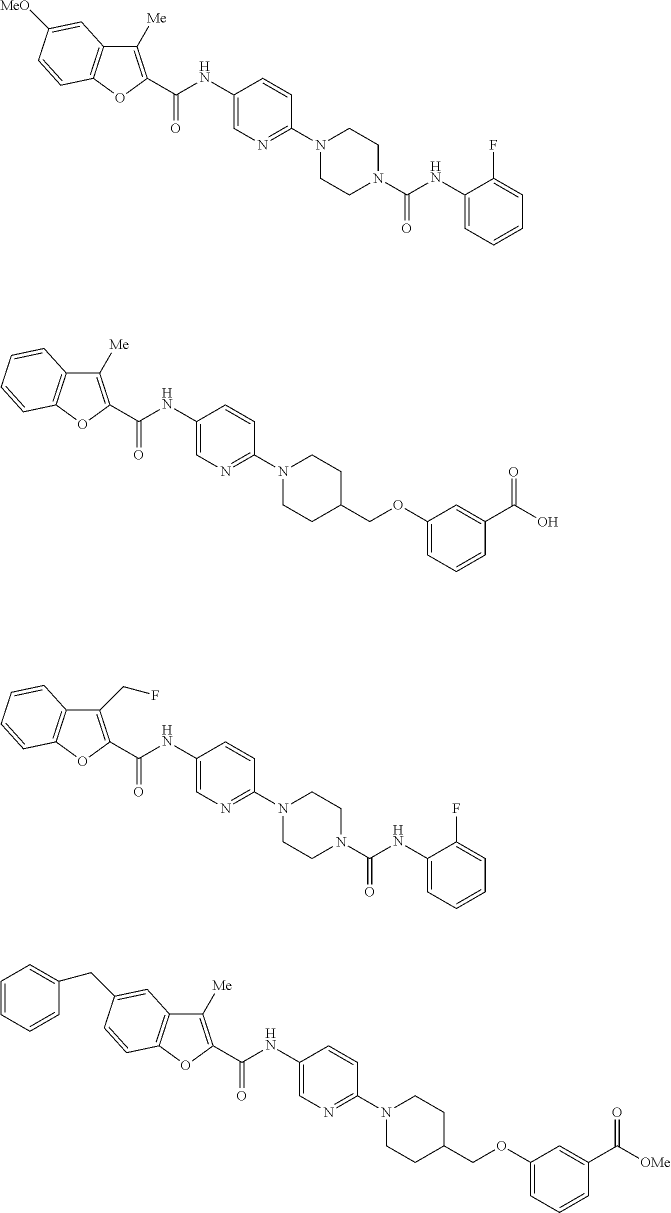

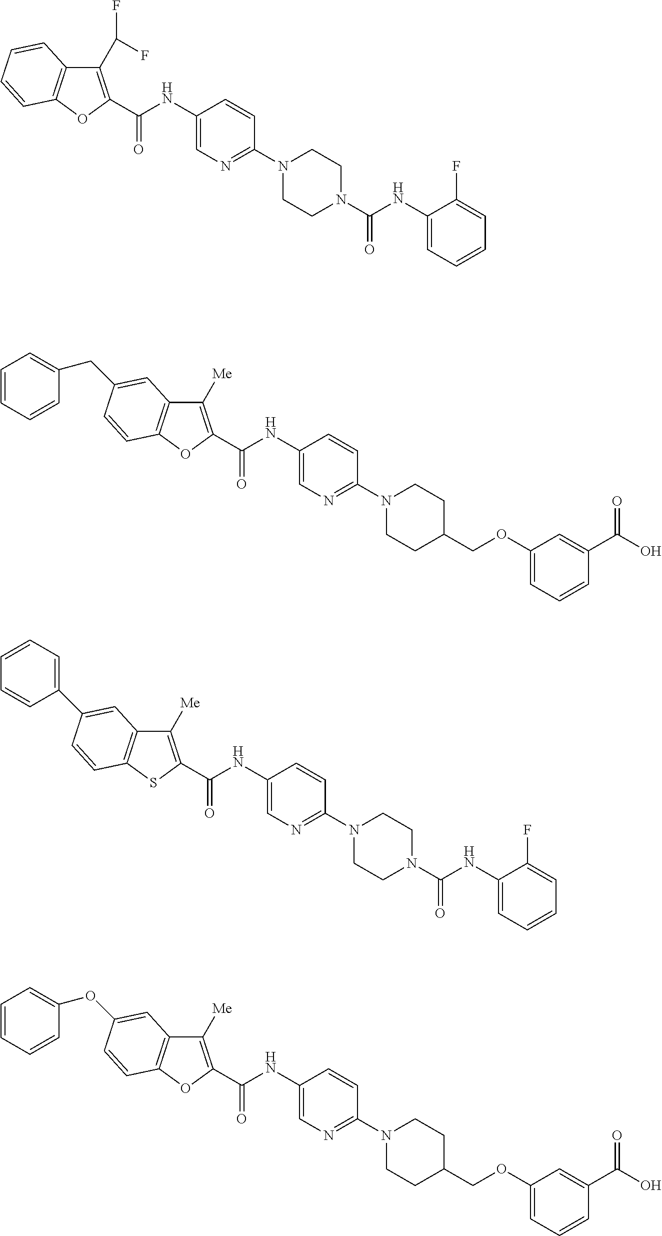

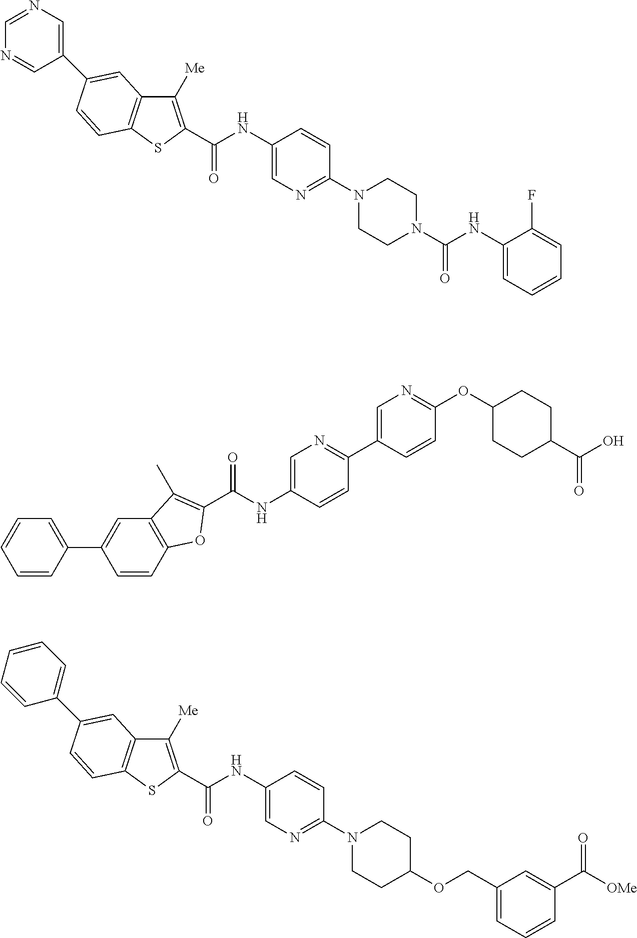

14. A pharmaceutical composition comprising a therapeutically effective amount of at least one compound of claim 1 and a pharmaceutically acceptable carrier.

15. A method of treating obesity, dyslipidemia, diabetes, impaired glucose tolerance or impaired fasting glucose in a patient, comprising administering to the patient a therapeutically effective amount of at least one compound of claim 1.

16. (canceled)

17. A compound or pharmaceutically acceptable salt of claim 1 wherein, Z is C(R³).
